(19) United States
(12) Patent Application Publication
(10) Pub. No.: US 2012/0282228 A1
(43) Pub. Date: Nov. 8, 2012

(54) METHOD OF PRODUCING PROGENITOR CELLS FROM DIFFERENTIATED CELLS

(76) Inventor: Vishal Bhasin, Wellington (NZ)

(21) Appl. No.: 13/384,068

(22) PCT Filed: Jul. 15, 2010
(86) PCT No.: PCT/AU2010/000901

§ 371 Date: Jul. 25, 2012

(30) Foreign Application Priority Data
Jul. 15, 2009 (AU) 2009903293
Sep. 22, 2009 (AU) 2009904597

(51) Int. Cl. C12N 5/071; A61K 35/12; C12N 5/0775

(52) U.S. Cl. 424/93.7; 435/377; 435/325; 435/395; 435/398

(57) ABSTRACT

The present invention provides a method of producing progenitor cells, such as cells capable of being differentiated into a plurality of different cell types, from differentiated cells. Methods of using progenitor cells in differentiation and/or tissue or organ repair and/or regeneration and/or building are also provides. Methods of using progenitor cells in treatment and prophylaxis of conditions alleviated by administering stem cells or tissue or organ derived from stem cells to a subject or by grafting stem cells or tissue or organ derived from stem cells into a subject or by transplanting stem cells or tissue or organ derived from stem cells into a subject are also provided. Also included are progenitor cells and differentiated cells and/or tissues and/or organs derived therefrom, and kits comprising same.

### RELATED APPLICATIONS

This application claims priority from Australian Patent Application Nos. AU 2009903293 filed Jul. 15, 2009 and AU 2009904597 filed Sep. 22, 2009, the contents of which are incorporated herein by reference in their entirety.

### FIELD OF THE INVENTION

The present invention is in the field of medicinal cell biology and more particularly to cell culture, especially the culture of primary cells and cell lines that are differentiated or terminally differentiated. The present invention also relates to methods for producing cells with the ability to differentiate into a plurality of cell types such as for use in medicine and/or veterinary applications and/or for animal improvement.

### BACKGROUND OF THE INVENTION

The utility of stem cells (SCs), including hematopoietic SCs, mesenchymal SCs or multipotent adult progenitor cells such as endothelial progenitor cells (EPCs) and embryonic stem cells (ESCs), is well established, especially for generating multiple distinct cell types in medicine and/or veterinary applications and/or for animal improvement.

In particular, stem cells may be used as a source of cells that can be differentiated into various cell types to repopulate damaged cells. For example, joint pain is a major cause of disability, which most often results from damage to the articular cartilage by trauma or degenerative joint diseases such as primary osteoarthritis. Current methods of treatment for cartilage damage are often not successful in regenerating cartilage tissue to a fully functional state, and there is often considerable donor-site rejection. A resolution of this disease state can be provided by regenerating cartilage tissue using stem cells. There are many other tissue degenerative diseases, which can be treated using stem cells, including autoimmune disorders. For example, in the treatment and/or therapy of diabetes, the pancreatic islet cells of a diabetic patient can be regenerated using stem cells that are implanted and/or infused into the patient.

Despite the pluripotency of embryonic stem (ES) cells, legal and moral controversies concerning their use, and the lack of available human ES lines, have prompted researchers to turn to investigating new sources for isolating stem cells from tissues that are not of fetal origin. However, such adult stem cells still involve complicated isolation procedures, and are in limited supply.

Because of the numerous obstacles and technical difficulties in producing and using ES cells and adult stem cells in sufficient quantity for a large number of clinical applications, many researchers are now looking to develop strategies to reprogram somatic cells from adult tissues to thereby create cells having stem cell-like attributes, in particular the ability to differentiate into different cell types.

In one approach, mature cells are fused with embryonic germ cells by a process known as somatic-cell nuclear transfer (SCNT). After fusion, mature cell nuclei display pluripotent properties similar to that of the embryonic germ cells (Tada et al., 1997, EMBO J. 16:6510-6520). This fusion-process essentially returns the mature adult cell to an earlier developmental state (immature state), from which the cell can then mature into differentiated cell types. However, such reprogramming does not escape the requirement for isolated ES-cells or embryonic germ cells. Moreover, the ethical and religious issues associated with using human embryos apply equally to this technology. There are also practical difficulties in SCNT, including the short supply of human oocytes for SCNT.

In another approach, the sequential exposure of primary oligodendrocyte precursor cells (OPCs) to fetal calf serum and basic fibroblast growth factor (bFGF) produces cells that resemble multipotent stem cells (Kondo et al., 2000, Science 289:1754-1757). However, the procedure has not been shown to be applicable to other cells types and, as OPCs are not an abundant cell type, there is limited prospect for the large-scale application of this technology.

Finally, human fibroblasts have been shown to be capable of being made into pluripotent cells by ectopic expression of four factors: Oct3/4, Sox2, Klf4, and c-Myc (Kzutoshi et al., Cell 131:861-872 (2007); Park et al., Nature epub (2007)). The so-called “induced pluripotent stem cells” (iPSCs) produced by this technology were shown to be similar to human embryonic stem (ES) cells in morphology, proliferation, surface antigens, gene expression, epigenetic status of pluripotent cell-specific genes, and telomerase activity. On the other hand, the iPSCs were also shown to give rise to teratomas, raising concerns about the application of the technology to medicine and/or the veterinary industry and/or for animal improvement.

Accordingly, there is a need in the art for an abundant source of cells that are capable of being differentiated into different cell types without extracting or using egg cells or stem cells such as ES cells or the like, and with minimal deleterious effects. More particularly, there is a need in the art for alternative and/or improved methods of culturing differentiated cells and culture media suitable for producing cells capable of differentiating into a plurality of cells types, and which are preferably capable of supporting self-renewal of cells having this capacity. There is also a need for culture systems that permit maintenance of cells capable of differentiating into a plurality of cells types in vitro until the cells are required for subsequent cell or tissue regeneration or repair.

### SUMMARY OF THE INVENTION

In work leading up to the present invention, the inventor sought to identify conditions for producing cells having the ability to differentiate into multiple cell types i.e., that could be used as a source of different cell types in a similar manner to mesenchymal stem cells. Against conventional wisdom in the art, the inventor reasoned that the de-differentiation of already differentiated cells might provide an abundant source of such cells for medical applications e.g., as an “off-the-shelf” supply of stem cell-like cells. The inventor also went against conventional wisdom in not merely seeking to expand existing populations of stems cells from primary tissues, by using differentiated cells as starting material.

The inventor has reasoned that it is possible to produce a cell having the ability to differentiate into a different cell type by culturing human fibroblasts in a medium comprising dexamethasone (DEX), compared to standard culture medium without dexamethasone (DEX). The inventor has also reasoned that it is possible to produce a cell having the ability to differentiate into a different cell type by culturing human fibroblasts in a medium comprising one or more other modulators of GTPase RhoA and/or a modulator of its down stream effector Rho-associated kinase or ROCK, compared to standard culture medium without one or more modulators of RhoA and/or without one or more modulators of ROCK. The inventor has also reasoned that it is possible to produce a cell having the ability to differentiate into a different cell type by culturing human fibroblasts in a medium comprising one or more modulators of a Sarcoma proto-oncogenic tyrosine kinase or SRC, compared to standard culture medium without one or more modulators of SRC.

As exemplified herein, it is possible to produce a cell having the ability to differentiate into a different cell type by culturing human fibroblasts in media comprising dexamethasone and/or one or more other modulators of the RhoA and/or ROCK pathway and/or media comprising one or more modulators of the SRC pathway, compared to standard culture media without dexamethasone and without one or more other modulators of RhoA and/or ROCK pathway and without one or more modulators of the SRC pathway. As exemplified herein, the human fibroblasts may be cultured simultaneously or sequentially media comprising dexamethasone or one or more other modulators of RhoA and/or ROCK pathway and media comprising one or more modulators of SRC to produce a cell having the ability to differentiate into a different cell type. Accordingly, as exemplified, it is possible to produce a cell having the ability to differentiate into a plurality of different cell types by culturing human fibroblasts in medium comprising dexamethasone and/or one or more other modulators of RhoA and/or ROCK pathway and also comprising one or more modulators of SRC pathway, compared to standard culture medium without dexamethasone or without one or more other modulators of RhoA and/or ROCK pathway, and without one or more modulators of SRC pathway.

Accordingly, in one example, the present invention provides a method for producing a progenitor cell that is capable of being differentiated into a plurality of different cell types, said method comprises incubating differentiated cells in a medium comprising dexamethasone, for a time and under conditions sufficient to render a differentiated cell capable of being differentiated into a plurality of different cell types. In one such example, dexamethasone is capable of inducing de-differentiation of the differentiated cells into the progenitor cells. In a further example, dexamethasone induces de-differentiation of the differentiated cells into the progenitor cells.

According to another example, the present invention provides a method for producing a progenitor cell that is capable of being differentiated into a plurality of different cell types, said method comprises incubating differentiated cells in a medium comprising one or more modulators of RhoA and/or ROCK pathway, for a time and under conditions sufficient to render a differentiated cell capable of being differentiated into a plurality of different cell types. In one such example, the one or more modulators of RhoA and/or ROCK pathway is/are capable of inducing de-differentiation of the differentiated cells into the progenitor cells. In a further example, the one or more modulators of RhoA and/or ROCK pathway induce de-differentiation of the differentiated cells into the progenitor cells.

According to another example, the present invention provides a method for producing a progenitor cell that is capable of being differentiated into a plurality of different cell types, said method comprises incubating differentiated cells in a medium comprising one or more modulators of SRC pathway, for a time and under conditions sufficient to render a differentiated cell capable of being differentiated into a plurality of different cell types. In one such example, the one or more modulators of SRC pathway is/are capable of inducing de-differentiation of the differentiated cells into the progenitor cells. In a further example, the one or more modulators of SRC pathway induce de-differentiation of the differentiated cells into the progenitor cells.

In one example, the present invention provides a method for producing a progenitor cell that is capable of being differentiated into a plurality of different cell types, said method comprises simultaneously or sequentially incubating differentiated cells in a medium comprising dexamethasone and/or one or more other modulators of RhoA and/or ROCK pathway and/or incubating differentiated cells in a medium comprising one or more modulators of SRC pathway for a time and under conditions sufficient to render a differentiated cell capable of being differentiated into a plurality of different cell types. Where a plurality of modulators is employed, it is within the scope of the invention to utilize those modulators simultaneously or sequentially in any order, or alternatively at the same time. The order of incubation in a plurality of modulators is not essential to the production of the progenitor cells of the invention that are capable of undergoing subsequent differentiation into a plurality of different cell types. Conveniently, differentiated cells are incubated in a medium comprising dexamethasone and/or one or more other modulators of RhoA and/or ROCK pathway in combination with one or more modulators of SRC pathway. Alternatively, the differentiated cells are incubated in a medium comprising a dexamethasone and/or one or more other modulators of RhoA and/or ROCK pathway and then incubated in a medium comprising one or more modulators of SRC pathway. Alternatively, the differentiated cells are incubated in a medium comprising one or more modulators of SRC pathway and then incubated in a medium comprising dexamethasone and/or one or more other modulators of RhoA and/or ROCK pathway.

It is also to be understood that the differentiated cells may be incubated separately in media comprising dexamethasone and media comprising one or more other modulators of RhoA and/or ROCK pathway, and that either or both of said media may also be supplemented with one or more other modulators employed in the inventive method e.g., one or more modulators of SRC pathway. In another example, the present invention provides a method of producing a progenitor cell that is capable of being differentiated into a plurality of different cell types, said method comprises simultaneously or sequentially incubating differentiated cells in a medium comprising dexamethasone and/or a medium comprising one or more other modulators of RhoA and/or ROCK pathway and/or a medium comprising one or more modulators of SRC pathway. In one such example, the differentiated cells are incubated in a medium comprising dexamethasone e.g., before incubation of the cells with one or more modulators of RhoA and/or ROCK pathway. Alternatively, or in addition, the differentiated cells are incubated in a medium comprising dexamethasone before incubation of the cells with one or more modulators of SRC pathway. Alternatively, the differentiated cells are incubated in a medium comprising one or more modulators of RhoA and/or ROCK pathway before incubation in the presence of dexamethasone and/or one or more modulators of SRC pathway. Alternatively, the differentiated cells are incubated in a medium comprising one or more modulators of SRC pathway before incubation in the presence of dexamethasone and/or one or more modulators of RhoA and/or ROCK pathway.

In the foregoing examples, the stated incubation(s) are separately or collectively sufficient to render a differentiated cell capable of being differentiated into a plurality of different cell types. Thus, the effect of combined modulators may be additive. The only requirement in such examples of the invention is that at least one modulator, e.g., one or two or three or four or five or more modulators, renders a differentiated cell capable of being differentiated into a plurality of different cell types.

In one example, the differentiated cells are incubated in the presence of one or more modulators of RhoA and/or ROCK according to any example hereof, for a time and under conditions sufficient to modulate a RhoA and/or ROCK pathway in the cells. In another example, the differentiated cells are incubated in the presence of one or more modulators of SRC according to any example hereof for a time and under conditions sufficient to modulate an SRC pathway in the cells.

In one example, cells are incubated in the presence of dexamethasone to achieve optimum plasticity and/or multipotency or pluripotency. Such incubation is preferably for a time and under conditions sufficient to render the cells capable of being differentiated into a plurality of different cell types. In one such example, the cells are incubated in the presence of dexamethasone for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division. Alternatively, or in addition, the cells are incubated in the presence of dexamethasone for a period of time sufficient for phosphorylation and/or activation and/or stabilization of tumor suppressor p53 protein that delays or inhibits or represses cell cycle progression or cell division. In one such example, differentiated cells are incubated in the presence of dexamethasone for a time and under conditions that is not sufficient to also activate an Akt/(PKB) pathway and/or an NF-κB pathway. Alternatively, or in addition, incubation of differentiated cells in the presence of dexamethasone does not agonise or partially agonise an Akt/(PKB) pathway and/or an NF-κB pathway. In one example, the differentiated cells are incubate in the presence of dexamethasone for a time and under conditions sufficient to modulate a RhoA and/or ROCK pathway in the cells but not sufficient to also activate an Akt/(PKB) pathway and/or an NF-κB pathway.

In one example, an active derivative or analogue of dexamethasone e.g., a steroid analogue is employed to render the cells capable of being differentiated into a plurality of different cell types. A skilled artisan can readily determine such active derivatives and/or analogues using procedures known in the art. In one example, active derivative of dexamethasone or their functional equivalent may be naturally occurring or isolated by means known to those skilled in the art, such as for example as described in Skrabalak et al., J. Pharmacological Methods 8(4): 291-297 (1982), or any reference describes therein, and is incorporated herein by reference. Alternatively , or in addition derivatives may be generated chemically or synthetically by several means known to those skilled in the art, for example as described by Arth et al., J. Am. Chem. Soc. 80: 3161 (1958) and Taub et al J. Am. Chem. Soc. 80: 4435 (1958).

As used herein the term “modulation of RhoA and/or ROCK pathway” or similar term shall be taken to mean modulation of the RhoA/Rho-associated kinase (ROCK) pathway, and to include modulation of RhoA and/or ROCK proteins and/or modulation of an interaction between RhoA and ROCK proteins and/or modulation of a downstream effect mediated by RhoA and/or ROCK proteins. In this respect, RhoA serves as a molecular switch by cycling between the inactive GDP-bound state and the active GTP-bound state, wherein the active conformation of RhoA interacts with its downstream target, the Rho-associated kinase (p160ROCK/Rho kinase) thereby mediating RhoA-mediated response(s).

In one example, a modulator of RhoA and/or ROCK pathway activates and/or enhances one or more functions of RhoA and/or ROCK protein(s) and/or activates and/or enhances RhoA and/or ROCK signalling. According to this example, the modulator comprises an agonist and/or a partial agonist and/or a reverse antagonist of RhoA and/or ROCK protein(s). For example, the cells are incubated in the presence of one or more modulators of RhoA and/or ROCK pathway to thereby achieve optimum plasticity and/or multipotency or pluripotency. Such incubation is preferably for a time and under conditions sufficient to induce and/or activate RhoA and/or ROCK pathway thereby rendering the cells capable of being differentiated into a plurality of different cell types.

In another example, a modulator of RhoA and/or ROCK pathway suppresses and/or inhibits one or more functions of RhoA and/or ROCK protein(s) and/or suppresses and/or inhibits RhoA and/or ROCK signalling. According to this example the modulator comprises an antagonist and/or a partial antagonist and/or a reverse agonist of RhoA and/or ROCK protein(s). For example, the cells are incubated in the presence of one or more modulators of RhoA and/or ROCK pathway for a time and under conditions sufficient to inhibit and/or suppress RhoA and/or ROCK pathway thereby rendering the cells capable of being differentiated into a plurality of different cell types.

Alternatively, or in addition, the cells are incubated in the presence of one or more modulators of RhoA and/or ROCK pathway for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division. Alternatively, or in addition, the cells are incubated in the presence of a modulator of RhoA and/or ROCK for a period of time sufficient for phosphorylation and/or activation and/or stabilization of tumor suppressor p53 protein to thereby delay or inhibit or repress cell cycle progression or cell division.

In one example, modulators of RhoA and/or ROCK pathway suitable for this purpose include but are not limited to dexamethasone (DEX) and/or growth hormone (GH) and/or tumor Necrosis factor-α (TNFα) and/or fibronectin and/or lysophosphatidic acid and/or serum and/or Y-27632 (e.g., Alexis Biochemicals) and/or any active fragment or any active chemical group thereof and any combination thereof. In one example, the differentiated cells are incubated in a medium comprising dexamethasone and/or GH and/or TNFα and/or fibronectin and/or lysophosphatidic acid and/or serum and/or Y-27632 and/or any active fragment or any active chemical group thereof and any combination thereof. In one preferred example, a modulator of RhoA and/or ROCK is dexamethasone.

In one example, the differentiated cells are incubate in the presence of one or more modulators of RhoA and/or ROCK pathway for a time and under conditions sufficient to modulate a RhoA and/or ROCK pathway in the cells but not sufficient to also activate an Akt/(PKB) pathway and/or an NF-κB pathway. In one such example, the one or more modulators of RhoA and/or ROCK do not agonise or partially agonise or does not antagonise or partially antagonise an Akt/(PKB) pathway and/or an NF-κB pathway.

As used herein the term “modulation of the SRC pathway” or similar term shall be taken to include modulation of a Src family member protein e.g., the protein tyrosine kinase c-Src or Fyn protein or Yes protein and/or modulation of an interaction between said Src family member protein and one or more molecules capable of activating the Src family member protein e.g., by binding to it and/or modulation of a downstream effect mediated by said Src family member protein. For example, “modulation of a SRC pathway” may comprise modulation of c-Src protein directly or indirectly e.g., by signalling from EGFR, VEGFR, PDGFR, or c-Met, to phosphorylate or dephosphorylate a cytoplasmic tyrosine kinase domain of c-Src, thereby modulating DNA synthesis and/or cell cycle progression and/or cell growth/proliferation. In one example, a modulator of SRC pathway activates and/or enhances a function of a Src family member protein, e.g., c-Src. According to this example, the modulator may include an agonist and/or a partial agonist and/or a reverse antagonist of the Src family member protein. In another example, a modulator of SRC suppresses and/or inhibits function of a Src family member protein, e.g., c-Src. According to this example the modulator may include an antagonist and/or a partial antagonist and/or a reverse agonist of the Src family member protein.

In one example, the cells are incubated in the presence of one or more modulators of the SRC pathway to achieve optimum plasticity and/or multipotency or pluripotency. Such incubation is preferably for a time and under conditions sufficient to induce and/or activate the SRC pathway or a component thereof that is sufficient to render the cells capable of being differentiated into a plurality of different cell types. Alternatively, the cells are incubated in the presence of one or more modulators of SRC pathway for a time and under conditions sufficient to inhibit and/or suppress SRC pathway or a component thereof that is sufficient to render the cells capable of being differentiated into a plurality of different cell types.

Alternatively, or in addition, the cells are incubated in the presence of one or more modulators of SRC pathway for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18. These proteins are expressed in fibroblasts and down-regulated before the onset of cell division. Alternatively, or in addition, the cells are incubated in the presence of one or more modulator of SRC pathway for a period of time sufficient for phosphorylation and/or activation and/or stabilization of tumor suppressor p53 protein that delays or inhibits or represses cell cycle progression or cell division.

In one example, modulators of SRC pathway suitable for this purpose are described herein and include but not limited to transforming growth factor (TGF) and/or transforming growth factor beta-1 (TGF-β1) and/or nerve growth factor beta (NGFβ) and/or interleukin 1-β (IL-1β) and/or Fibroblast growth factor-1 (FGF-1) and/or fibroblast growth factor-2 (FGF-2) and/or hepatocyte growth factor (HGF) and/or neurotrophin 3 (NT3) and/or a semaphorin (SEMA) such as SEMA-3A and/or platelet-derived growth factor (PDGF) such as platelet-derived growth factor B-chain (PDGF-B) and/or 4-amino-5-(4-methylphenyl)-7-(t-butyl)pyrazolo (3,4-d) pyrimidine, (PP1) and/or 4-Amino-5-(4-chlorophenyl)-7-(t-butyl)pyrazolo[3,4-d]pyrimidine (PP2) and/or SU6656 and/or UCS15A or any active fragment or any active chemical group thereof, and any combination thereof. In one example, the differentiated cells are incubated in a medium comprising TGF and/or TGF-β1 and/or NGFβ and/or IL-1β and/or FGF-1 and/or FGF-2 and/or HGF and/or NT3 and/or SEMA such as SEMA-3A and/or PDGF such as PDGF-B and/or any active fragment or any active chemical group thereof.

In one example, the differentiated cells are incubate in the presence of one or more modulators of SRC pathway for a time and under conditions sufficient to modulate the SRC pathway in the cells but not sufficient to also activate an Akt/(PKB) pathway and/or an NF-κB pathway. In one such example, a modulator of SRC pathway does not agonise or partially agonise and/or antagonise or partially antagonise an Akt/(PKB) pathway and/or an NF-κB pathway.

In one example, the present invention provides a method for producing a progenitor cell that is capable of being differentiated into a plurality of different cell types, said method comprises simultaneously or sequentially incubating differentiated cells in a medium comprising dexamethasone and/or one or more modulators of RhoA and/or ROCK pathway and a medium comprising one or more modulators of SRC pathway, for a time and under conditions sufficient to render a differentiated cell capable of being differentiated into a plurality of different cell types. The one or more modulators of RhoA and/or ROCK pathway may be selected from one or more of dexamethasone and/or GH and/or TNFα and/or fibronectin and/or lysophosphatidic acid and/or serum and/or and Y-27632 and/or any active fragment or any active chemical group thereof and any combination thereof. The one or more modulators of SRC pathway may be selected from one or more of TGF-β1 and/or NGF and/or IL-10 and/or FGF-1 and/or FGF-2 and/or HGF and/or NT3 and/or SEMA-3A and/or PDGF such PDGF-B and/or PP1 and/or PP2 and/or SU6656 and/or UCS15A or any active fragment or any active chemical group thereof, and in any combination thereof. In one example, the modulator of RhoA and/or ROCK is dexamethasone or an active fragment or derivative or an active chemical group thereof. In one example, dexamethasone or any other modulator of RhoA and/or ROCK pathway does not agonise or partially agonise and/or antagonise or partially antagonise an Akt/(PKB) pathway and/or an NF-κB pathway. In another example, the modulator of SRC pathway does not agonise or partially agonise and/or antagonise or partially antagonise an Akt/(PKB) pathway and/or an NF-κB pathway.

In one example, the differentiated cells are incubated in a medium comprising HGF and further incubated in a medium comprising dexamethasone. Alternatively, the differentiated cells are incubated in a medium comprising dexamethasone and further incubated in a medium comprising HGF. Alternatively, or in addition, the differentiated cells are incubated in a medium comprising both HGF and dexamethasone. According to this example, incubation of the differentiated cells in the presence of HGF and/or dexamethasone is for a time and under conditions sufficient to render a differentiated cell capable of being differentiated into a plurality of different cell types.

In another example, differentiated cells are incubated in a medium comprising IL-1β and further incubated in a medium comprising dexamethasone. Alternatively, the differentiated cells are incubated in a medium comprising dexamethasone and further incubated in a medium comprising IL-β. Alternatively, or in addition, the differentiated cells are incubated in a medium comprising both IL-1β and dexamethasone. According to this example, incubation of the differentiated cells in the presence of IL-1β and/or dexamethasone is for a time and under conditions sufficient to render a differentiated cell capable of being differentiated into a plurality of different cell types.

In another example, differentiated cells are incubated in a medium comprising FGF-1 and further incubated in a medium comprising dexamethasone. Alternatively, the differentiated cells are incubated in a medium comprising dexamethasone and further incubated in a medium comprising FGF-1. Alternatively, or in addition, the differentiated cells are incubated in a medium comprising both FGF-1 and dexamethasone. According to this example, incubation of the differentiated cells in the presence of FGF-1 and/or dexamethasone is for a time and under conditions sufficient to render a differentiated cell capable of being differentiated into a plurality of different cell types.

In another example, differentiated cells are incubated in a medium comprising FGF-2 and further incubated in a medium comprising dexamethasone. Alternatively, the differentiated cells are incubated in a medium comprising dexamethasone and further incubated in a medium comprising FGF-2. Alternatively, or in addition, the differentiated cells are incubated in a medium comprising both FGF-2 and dexamethasone. According to this example, incubation of the differentiated cells in the presence of FGF-2 and/or dexamethasone is for a time and under conditions sufficient to render a differentiated cell capable of being differentiated into a plurality of different cell types.

In another example, differentiated cells are incubated in a medium comprising NGFβ and further incubated in a medium comprising dexamethasone. Alternatively, the differentiated cells are incubated in a medium comprising dexamethasone and further incubated in a medium comprising NGFβ. Alternatively, or in addition, the differentiated cells are incubated in a medium comprising both NGFβ and dexamethasone. According to this example, incubation of the differentiated cells in the presence of NGFβ and/or dexamethasone is for a time and under conditions sufficient to render a differentiated cell capable of being differentiated into a plurality of different cell types.

In one example, the differentiated cells are incubated in a medium comprising HGF and further incubated in a medium comprising GH. Alternatively, the differentiated cells are incubated in a medium comprising GH and further incubated in a medium comprising HGF. Alternatively, or in addition, the differentiated cells are incubated in a medium comprising both HGF and GH. According to this example, incubation of the differentiated cells in the presence of HGF and/or GH is for a time and under conditions sufficient to render a differentiated cell capable of being differentiated into a plurality of different cell types.

In another example, differentiated cells are incubated in a medium comprising IL-1β and further incubated in a medium comprising GH. Alternatively, the differentiated cells are incubated in a medium comprising GH and further incubated in a medium comprising IL-1β. Alternatively, or in addition, the differentiated cells are incubated in a medium comprising both IL-1β and GH. According to this example, incubation of the differentiated cells in the presence of IL-1β and/or GH is for a time and under conditions sufficient to render a differentiated cell capable of being differentiated into a plurality of different cell types.

In another example, differentiated cells are incubated in a medium comprising FGF-1 and further incubated in a medium comprising GH. Alternatively, the differentiated cells are incubated in a medium comprising GH and further incubated in a medium comprising FGF-1. Alternatively, or in addition, the differentiated cells are incubated in a medium comprising both FGF-1 and GH. According to this example, incubation of the differentiated cells in the presence of FGF-1 and/or GH is for a time and under conditions sufficient to render a differentiated cell capable of being differentiated into a plurality of different cell types.

In another example, differentiated cells are incubated in a medium comprising FGF-2 and further incubated in a medium comprising GH. Alternatively, the differentiated cells are incubated in a medium comprising GH and further incubated in a medium comprising FGF-2. Alternatively, or in addition, the differentiated cells are incubated in a medium comprising both FGF-2 and GH. According to this example, incubation of the differentiated cells in the presence of FGF-2 and/or GH is for a time and under conditions sufficient to render a differentiated cell capable of being differentiated into a plurality of different cell types.

In another example, differentiated cells are incubated in a medium comprising NGFβ and further incubated in a medium comprising GH. Alternatively, the differentiated cells are incubated in a medium comprising GH and further incubated in a medium comprising NGFβ. Alternatively, or in addition, the differentiated cells are incubated in a medium comprising both NGFβ and GH. According to this example, incubation of the differentiated cells in the presence of NGF and/or GH is for a time and under conditions sufficient to render a differentiated cell capable of being differentiated into a plurality of different cell types.

In one example, the differentiated cells are incubated in a medium comprising HGF and further incubated in a medium comprising TNFα. Alternatively, the differentiated cells are incubated in a medium comprising TNFα and further incubated in a medium comprising HGF. Alternatively, or in addition, the differentiated cells are incubated in a medium comprising both HGF and TNFα. According to this example, incubation of the differentiated cells in the presence of HGF and/or TNFα is for a time and under conditions sufficient to render a differentiated cell capable of being differentiated into a plurality of different cell types.

In another example, differentiated cells are incubated in a medium comprising IL-1β and further incubated in a medium comprising TNFα. Alternatively, the differentiated cells are incubated in a medium comprising TNFα and further incubated in a medium comprising IL-1β. Alternatively, or in addition, the differentiated cells are incubated in a medium comprising both IL-1β and TNFα. According to this example, incubation of the differentiated cells in the presence of IL-1β and/or TNFα is for a time and under conditions sufficient to render a differentiated cell capable of being differentiated into a plurality of different cell types.

In another example, differentiated cells are incubated in a medium comprising FGF-1 and further incubated in a medium comprising TNFα. Alternatively, the differentiated cells are incubated in a medium comprising TNFα and further incubated in a medium comprising FGF-1. Alternatively, or in addition, the differentiated cells are incubated in a medium comprising both FGF-1 and TNFα. According to this example, incubation of the differentiated cells in the presence of FGF-1 and/or TNFα is for a time and under conditions sufficient to render a differentiated cell capable of being differentiated into a plurality of different cell types.

In another example, differentiated cells are incubated in a medium comprising FGF-2 and further incubated in a medium comprising TNFα. Alternatively, the differentiated cells are incubated in a medium comprising TNFα and further incubated in a medium comprising FGF-2. Alternatively, or in addition, the differentiated cells are incubated in a medium comprising both FGF-2 and TNFα. According to this example, incubation of the differentiated cells in the presence of FGF-2 and/or TNFα is for a time and under conditions sufficient to render a differentiated cell capable of being differentiated into a plurality of different cell types.

In another example, differentiated cells are incubated in a medium comprising NGFβ and further incubated in a medium comprising TNFα. Alternatively, the differentiated cells are incubated in a medium comprising TNFα and further incubated in a medium comprising NGFβ. Alternatively, or in addition, the differentiated cells are incubated in a medium comprising both NGFβ and TNFα. According to this example, incubation of the differentiated cells in the presence of NGFβ and/or TNFα is for a time and under conditions sufficient to render a differentiated cell capable of being differentiated into a plurality of different cell types.

In one example, the differentiated cells are incubated in a medium comprising HGF and further incubated in a medium comprising Fibronectin. Alternatively, the differentiated cells are incubated in a medium comprising Fibronectin and further incubated in a medium comprising HGF. Alternatively, or in addition, the differentiated cells are incubated in a medium comprising both HGF and Fibronectin. According to this example, incubation of the differentiated cells in the presence of HGF and/or Fibronectin is for a time and under conditions sufficient to render a differentiated cell capable of being differentiated into a plurality of different cell types.

In another example, differentiated cells are incubated in a medium comprising IL-113 and further incubated in a medium comprising Fibronectin. Alternatively, the differentiated cells are incubated in a medium comprising Fibronectin and further incubated in a medium comprising IL-1β. Alternatively, or in addition, the differentiated cells are incubated in a medium comprising both IL-1β and Fibronectin. According to this example, incubation of the differentiated cells in the presence of IL-1β and/or Fibronectin is for a time and under conditions sufficient to render a differentiated cell capable of being differentiated into a plurality of different cell types.

In another example, differentiated cells are incubated in a medium comprising FGF-1 and further incubated in a medium comprising Fibronectin. Alternatively, the differentiated cells are incubated in a medium comprising Fibronectin and further incubated in a medium comprising FGF-1. Alternatively, or in addition, the differentiated cells are incubated in a medium comprising both FGF-1 and Fibronectin. According to this example, incubation of the differentiated cells in the presence of FGF-1 and/or Fibronectin is for a time and under conditions sufficient to render a differentiated cell capable of being differentiated into a plurality of different cell types.

In another example, differentiated cells are incubated in a medium comprising FGF-2 and further incubated in a medium comprising Fibronectin. Alternatively, the differentiated cells are incubated in a medium comprising Fibronectin and further incubated in a medium comprising FGF-2. Alternatively, or in addition, the differentiated cells are incubated in a medium comprising both FGF-2 and Fibronectin. According to this example, incubation of the differentiated cells in the presence of FGF-2 and/or Fibronectin is for a time and under conditions sufficient to render a differentiated cell capable of being differentiated into a plurality of different cell types.

In another example, differentiated cells are incubated in a medium comprising NGFβ and further incubated in a medium comprising Fibronectin. Alternatively, the differentiated cells are incubated in a medium comprising Fibronectin and further incubated in a medium comprising NGFβ. Alternatively, or in addition, the differentiated cells are incubated in a medium comprising both NGF and Fibronectin. According to this example, incubation of the differentiated cells in the presence of NGF and/or Fibronectin is for a time and under conditions sufficient to render a differentiated cell capable of being differentiated into a plurality of different cell types.

In one example, the differentiated cells are incubated in a medium comprising HGF and further incubated in a medium comprising lysophosphatidic acid. Alternatively, the differentiated cells are incubated in a medium comprising lysophosphatidic acid and further incubated in a medium comprising HGF. Alternatively, or in addition, the differentiated cells are incubated in a medium comprising both HGF and lysophosphatidic acid. According to this example, incubation of the differentiated cells in the presence of HGF and/or lysophosphatidic acid is for a time and under conditions sufficient to render a differentiated cell capable of being differentiated into a plurality of different cell types.

In another example, differentiated cells are incubated in a medium comprising IL-1β and further incubated in a medium comprising lysophosphatidic acid. Alternatively, the differentiated cells are incubated in a medium comprising lysophosphatidic acid and further incubated in a medium comprising. Alternatively, or in addition, the differentiated cells are incubated in a medium comprising both IL-1β and lysophosphatidic acid. According to this example, incubation of the differentiated cells in the presence of IL-1β and/or lysophosphatidic acid is for a time and under conditions sufficient to render a differentiated cell capable of being differentiated into a plurality of different cell types.

In another example, differentiated cells are incubated in a medium comprising FGF-1 and further incubated in a medium comprising lysophosphatidic acid. Alternatively, the differentiated cells are incubated in a medium comprising lysophosphatidic acid and further incubated in a medium comprising FGF-1. Alternatively, or in addition, the differentiated cells are incubated in a medium comprising both FGF-1 and lysophosphatidic acid. According to this example, incubation of the differentiated cells in the presence of FGF-1 and/or lysophosphatidic acid is for a time and under conditions sufficient to render a differentiated cell capable of being differentiated into a plurality of different cell types.

In another example, differentiated cells are incubated in a medium comprising FGF-2 and further incubated in a medium comprising lysophosphatidic acid. Alternatively, the differentiated cells are incubated in a medium comprising lysophosphatidic acid and further incubated in a medium comprising FGF-2. Alternatively, or in addition, the differentiated cells are incubated in a medium comprising both FGF-2 and lysophosphatidic acid. According to this example, incubation of the differentiated cells in the presence of FGF-2 and/or lysophosphatidic acid is for a time and under conditions sufficient to render a differentiated cell capable of being differentiated into a plurality of different cell types.

In another example, differentiated cells are incubated in a medium comprising NGFβ and further incubated in a medium comprising lysophosphatidic acid. Alternatively, the differentiated cells are incubated in a medium comprising lysophosphatidic acid and further incubated in a medium comprising NGFβ. Alternatively, or in addition, the differentiated cells are incubated in a medium comprising both NGFβ and lysophosphatidic acid. According to this example, incubation of the differentiated cells in the presence of NGFβ and/or lysophosphatidic acid is for a time and under conditions sufficient to render a differentiated cell capable of being differentiated into a plurality of different cell types.

In one example, the differentiated cells are incubated in a medium comprising HGF and further incubated in a medium comprising Y-27632. Alternatively, the differentiated cells are incubated in a medium comprising Y-27632 and further incubated in a medium comprising HGF. Alternatively, or in addition, the differentiated cells are incubated in a medium comprising both HGF and Y-27632. According to this example, incubation of the differentiated cells in the presence of HGF and/or Y-27632 is for a time and under conditions sufficient to render a differentiated cell capable of being differentiated into a plurality of different cell types.

In another example, differentiated cells are incubated in a medium comprising IL-1β and further incubated in a medium comprising Y-27632. Alternatively, the differentiated cells are incubated in a medium comprising Y-27632 and further incubated in a medium comprising IL-113. Alternatively, or in addition, the differentiated cells are incubated in a medium comprising both IL-1β and Y-27632. According to this example, incubation of the differentiated cells in the presence of IL-1β and/or Y-27632 is for a time and under conditions sufficient to render a differentiated cell capable of being differentiated into a plurality of different cell types.

In another example, differentiated cells are incubated in a medium comprising FGF-1 and further incubated in a medium comprising Y-27632. Alternatively, the differentiated cells are incubated in a medium comprising Y-27632 and further incubated in a medium comprising FGF-1. Alternatively, or in addition, the differentiated cells are incubated in a medium comprising both FGF-1 and Y-27632. According to this example, incubation of the differentiated cells in the presence of FGF-1 and/or Y-27632 is for a time and under conditions sufficient to render a differentiated cell capable of being differentiated into a plurality of different cell types.

In another example, differentiated cells are incubated in a medium comprising FGF-2 and further incubated in a medium comprising Y-27632. Alternatively, the differentiated cells are incubated in a medium comprising Y-27632 and further incubated in a medium comprising FGF-2. Alternatively, or in addition, the differentiated cells are incubated in a medium comprising both FGF-2 and Y-27632. According to this example, incubation of the differentiated cells in the presence of FGF-2 and/or Y-27632 is for a time and under conditions sufficient to render a differentiated cell capable of being differentiated into a plurality of different cell types.

In another example, differentiated cells are incubated in a medium comprising NGFβ and further incubated in a medium comprising Y-27632. Alternatively, the differentiated cells are incubated in a medium comprising Y-27632 and further incubated in a medium comprising NGFβ. Alternatively, or in addition, the differentiated cells are incubated in a medium comprising both NGF and Y-27632. According to this example, incubation of the differentiated cells in the presence of NGFβ and/or Y-27632 is for a time and under conditions sufficient to render a differentiated cell capable of being differentiated into a plurality of different cell types.

In a preferred example, the differentiated cells are incubated in a medium comprising dexamethasone and/or HGF and/or IL-1β and/or FGF-1. Preferably, the differentiated cells are incubated in a medium comprising HGF and dexamethasone, or IL-1β and dexamethasone, or FGF-1 and dexamethasone under conditions sufficient to render a differentiated cell capable of being differentiated into a plurality of different cell types.

In one example, the method of the present invention according to any example hereof, may further comprise detaching the cells e.g., by incubating the cells in detachment medium containing a protease or a ligand of protease activated receptor (PAR). That is, in one example, the present invention provides a method for producing a progenitor cell that is capable of being differentiated into plurality of different cell types, said method comprising incubating differentiated cells in a medium comprising dexamethasone and/or one or more other modulators of RhoA and/or ROCK pathway and/or one or more modulators of SRC pathway and optionally detaching the cells e.g., by incubating the cells in detachment medium containing a protease or a ligand of protease activated receptor (PAR). In one such example, if cells are detached then the progenitor cells produced by this method are capable of being differentiated into plurality of different cell types until reattachment or adherence of the cells to the culture vessel and/or to each other.

The order of incubation in the presence dexamethasone and/or one or more other modulators of RhoA and/or ROCK pathway and/or incubation in the presence of one or more modulators of SRC pathway and detachment is not necessarily essential to the production of cells capable of undergoing subsequent differentiation into a plurality of different cell types. For example, the differentiated cells are incubated in a medium comprising dexamethasone before detachment. Alternatively, or in addition, the differentiated cells are incubated in a medium comprising one or more modulators of RhoA and/or ROCK pathway before detachment. Alternatively, or in addition, the differentiated cells are incubated in a medium comprising one or more modulators of SRC pathway before detachment. Conveniently, the differentiated cells are incubated in a medium comprising dexamethasone and/or one or more other modulators of RhoA and/or ROCK pathway and also comprising one or more modulators of SRC pathway, before performing detachment. Alternatively, the differentiated cells are incubated in a medium comprising dexamethasone and/or one or more other modulators of RhoA and/or ROCK pathway and/or one or more modulators of SRC pathway after performing detachment.

As used herein, the term “detachment” or variations such as “detaching the cells” or “cell detachment” shall be taken to include any method of detaching cells from each other and/or from a surface of a culture vessel in which they are maintained known in the art. In one example, the cells are incubated in a detachment medium comprising a protease or PAR ligand for a time and under conditions sufficient for the cells to detach from each other and/or from a surface of a culture vessel in which they are maintained or to become rounder in appearance. By “PAR ligand” or equivalent term is meant a ligand capable of activating a protease-activated receptor, such as PAR-1 and/or PAR-2 and/or PAR-3 and/or PAR4. Without being bound by any theory or mode of action, incubation in a protease or PAR ligand for a time and under conditions sufficient to detach the cells, or for their appearance to be modified in this manner, is sufficient for a partial or complete breakdown of integrins that normally mediate cell adhesion or at least for the promotion of cellular signalling pathways mediated by an integrin.

Alternatively, or in addition, the cells are incubated in a detachment medium comprising a protease or PAR ligand for a time and under conditions sufficient for activation of one or more protease-activated receptors (PARs) such as PAR-1 and/or PAR-2 and/or PAR-3 and/or PAR4 to occur.

Preferred proteases and PAR ligands for performing the invention include chymotrypsin, trypsin, thrombin, pepsin, papain, matrix-metalloproteinase (MMP) and a PAR-2-activating peptide comprising the sequence SLIGRL. More preferably, the protease is trypsin, thrombin, plasmin, or a PAR-2-activating peptide comprising the sequence SLIGRL. In a particularly preferred example, trypsin is employed.

In another example, the cells are detached from each other and/or from a surface of a culture vessel in which they are maintained or become rounder in appearance by incubating the cells in a Ca²⁺-free and Mg⁺-free detachment medium comprising ethylenediaminetetraacetic acid (EDTA) for a time and under conditions sufficient for detachment of integrins from the cellular matrix. In a further example, cells are detached from each other and/or from a surface of a culture vessel in which they are maintained or become rounder in appearance by incubating the cells in a detachment medium comprising citric saline for a time and under conditions sufficient for detachment of the cells and/or integrins from the cellular matrix.

In one example the method of the present invention optionally further comprises incubating differentiated cells in a medium comprising a low serum concentration and without supplementation of factors normally present in serum. The order of incubating differentiated cells in a medium comprising dexamethasone and/or one or more other modulators of RhoA and/or ROCK pathway and/or one or more modulators of SRC pathway, optionally detachment, and incubation in low serum medium is not necessarily essential to the production of cells capable of undergoing subsequent differentiation into a plurality of different cell types. Conveniently, the differentiated cells are incubated in a medium comprising dexamethasone and low serum concentration and without supplementation of factors normally present in serum. Alternatively, or in addition, the differentiated cells are incubated in a medium comprising one or more other modulators of RhoA and/or ROCK pathway and low serum concentration and without supplementation of factors normally present in serum. Alternatively, or in addition, the differentiated cells are incubated in a medium comprising one or more modulators of SRC pathway and low serum concentration and without supplementation of factors normally present in serum. Alternatively, or in addition, the differentiated cells are incubated in a medium comprising dexamethasone and/or one or more other modulators of RhoA and/or ROCK pathway and one or more modulators of SRC pathway and low serum concentration and without supplementation of factors normally present in serum. If cells are also detached from each other and/or from a surface of a culture vessel in which they are maintained, then conveniently cells may be incubated in presence of dexamethasone and/or one or more other modulators of RhoA and/or ROCK pathway and/or one or more modulators of SRC pathway as described above before performing detachment.

In one example, incubation of differentiated cells in a medium comprising low serum concentration and without supplementation of factors normally present in serum in concert with incubation of cells in presence of dexamethasone and/or one or more other modulators of RhoA and/or ROCK pathway, and/or one or more modulators of SRC pathway enhances proportion of progenitor cells capable of being differentiated into a plurality of different cell types that is being produced.

The term “serum” means the non-cellular liquid phase of blood that remains after coagulation and removal of the blood clot, including blood cells, platelets and fibrinogen. The present invention is not to be limited by the nature of the serum used in low-serum media, the only requirement being that the cells are able to maintain viability in the medium used. In this respect, it is known that normal human fibroblasts require growth factors provided e.g., by fetal bovine serum (FBS) or fetal calf serum (FCS) at about 10% (v/v) for proliferation in culture.

Preferred sera for cell culture are bovine sera e.g., fetal calf serum and fetal bovine serum. Horse sera or artificial sera comprising the constituents of naturally-occurring sera from these sources may also be employed. In another example, in using the cells of the invention in human therapy, preferred sera for cell culture are human sera or artificial sera comprising the constituents of naturally-occurring human sera.

As used herein, the term “low serum concentration” shall be taken to mean a concentration of serum not exceeding about 3% (v/v) in culture medium, preferably not exceeding about 2% (v/v) or about 1% (v/v), and still more preferably, less than 1% (v/v) serum concentration, including serum-free or no serum. Unless the context requires otherwise e.g., by virtue of the addition of a growth factor agonist of the Akt/(PKB) pathway and/or NF-κB pathway, the term “low-serum” shall also be taken to mean conditions in which the concentration of a growth factor supplement in the culture medium is at a level equivalent to or less than the level of the growth factor in serum. In the present context, an alternative low-serum medium includes “artificial sera” or “depleted sera” having low levels of growth factors required for cellular proliferation.

In a particularly preferred example, the term “low serum concentration” shall be taken to mean a concentration of serum between about 0% (v/v) and about 1% serum concentration or an artificial serum or depleted serum having an equivalent or lower level of one or more serum growth factors. Standard methods in cell biology are used to . determine the parameters for what constitutes a particular concentration of any serum, including fetal calf serum and bovine serum.

In one example, low-serum media for incubation of the differentiated cells are Dulbecco's Modified Eagle Medium High Glucose (DMEM-HG; e.g., Lonza Cat #12-604), or basal Medium or a modified Medium 199 containing high glucose. Preferably, the low-serum medium comprises one or more sugars such as glucose, at a concentration of at least about 0.1% (w/v), more preferably at least about 0.2% (w/v) or at least about 0.3% (w/v) or at least about 0.4% (w/v) or at least about 0.5% (w/v) or at least about 0.6% (w/v) or at least about 0.7% (w/v) or at least about 0.8% (w/v) or at least about 0.9% (w/v) or at least about 1.0% (w/v).

In one example, the differentiated cells are incubated in low-serum media for at least about two days i.e., about 48 hours, and not exceeding about ten days i.e., about 240 hours, including for about two days or about three days or about four days or about five days or about six days or about seven days or about eight days or about nine days or about ten days. More preferably, the cells are incubated in low-serum media for a period between about four days and about nine days, including about four days or about five days or about six days or about seven days or about eight days or about nine days. Still more preferably, the cells are incubated in low-serum media for a period between about five days and about eight days, including about five days or about six days or about seven days or about eight days. As will be apparent from the disclosure herein, lower numbers of progenitor cells may be apparent with shorter periods of exposure of the cells to low serum media than are observed for optimum periods of incubation in low serum media, however such sub-optimum incubation conditions are clearly within the scope of the invention.

Alternatively, or in addition, the cells are incubated in low-serum medium for a period of time sufficient for the level of one or more gene products of the cells that delay or inhibit or repress cell cycle progression or cell division to be expressed de novo or at an increased level in the cells, such as, for example, the cell cycle proteins p27Kip1 and/or p57Kip2 and/or p18.

In one example, the method of the present invention according to any example hereof further comprises incubating the cells under high cell-density conditions. In accordance with this example, a high density plating medium is employed. As used herein, the term “high density plating medium” means any cell medium capable of supporting progenitor cells produced by the method of the present invention. In one example progenitor cells produced by the method of the invention undergo minimal or no cell division when cultured, maintained or incubated in high density plating medium. Exemplary high density plating medium includes Medium-199 comprising 170 nM insulin, 0.5 mM 3-isobutyl-1-methylxanthine, 0.2 mM indomethacin, and 15% rabbit serum. Alternatively, high density plating medium includes Dulbecco's Modified Eagle Medium (DMEM) or basal Medium 199 supplemented with 10% fetal calf serum (FCS). However other media may be employed.

The order of incubation of differentiated in a medium comprising dexamethasone and/or one or more other modulators of RhoA and/or ROCK pathway and/or one of more modulators of SRC pathway and/or and optionally detachment and optionally incubating the cells in low-serum medium, and optionally incubating the cells under high cell density conditions is not necessarily essential to the production of progenitor cells capable of undergoing subsequent differentiation into a plurality of different cell types. For example, the differentiated cells are incubated in the presence dexamethasone optionally in a low-serum media, and subjected to one or more means of achieving their detachment, before being incubated under high-cell density conditions. Alternatively, or in addition, the differentiated cells are incubated in the presence of one or more modulators of RhoA and/or ROCK pathway and optionally in a low-serum media, and subjected to one or more means of achieving their detachment, before being incubated under high-cell density conditions. Alternatively, the differentiated cells are incubated in the presence of one or more modulators of SRC pathway and optionally in a low-serum media, and subjected to one or more means of achieving their detachment, before being incubated under high-cell density conditions. In such example, the differentiated cells are incubated in a medium comprising dexamethasone and/or one or more other modulators of RhoA and/or ROCK pathway and one or more modulators of SRC pathway and optionally incubated in a low-serum media, and subjected to one or more means of achieving their detachment, before being incubated under high-cell density conditions.

Alternatively, the differentiated cells are incubated in the presence dexamethasone under high cell density conditions optionally in a low-serum media, before being subjected to one or more means of achieving their detachment. Alternatively, or in addition, differentiated cells are incubated in the presence one or more modulators of RhoA and/or ROCK pathway under high cell density conditions optionally in a low-serum media, before being subjected to one or more means of achieving their detachment. Alternatively, or in addition, differentiated cells are incubated in presence of one or more modulators of SRC pathway under high cell density conditions optionally in a low-serum media, before being subjected to one or more means of achieving their detachment. In one example, the differentiated cells are incubated under high cell density conditions in a medium comprising dexamethasone or one or more other modulators of RhoA and/or ROCK pathway and one or more modulators of SRC pathway optionally in a low-serum media, before being subjected to one or more means of achieving their detachment.

An advantage of incubating cells at high cell density conditions in concert with incubation in the presence of dexamethasone or one or more modulators of RhoA and/or ROCK and/or one or more modulators of SRC is that the proportion of progenitor cells capable of being differentiated into a plurality of different cell types is increased.

As used herein, the term “high density” or similar term such as “high density conditions” or “high cell density conditions” shall be taken to mean that the cells are maintained, cultured or incubated until confluence or cell-to-cell contact is achieved or at a starting density of cells of about 50,000 cells to about 200,000 cells per standard-size culture well/plate, including about 60,000 cells or greater per standard-size culture well/plate, or about 70,000 cells or greater per standard-size culture well/plate, or about 80,000 cells or greater per standard-size culture well/plate, or about 90,000 cells or greater per standard-size culture well/plate, or about 100,000 cells or greater per standard-size culture well/plate, or about 200,000 cells per standard-size culture well/plate. Higher cell densities above about 200,000 cells per standard-size culture well/plate may also be employed. By “standard-size” in this context is meant about 27 mm² plating surface area in a well or plate.

Alternatively or in addition, high density conditions include the maintenance, culture or incubation of cells at a starting density of cells of about 1500 cells/mm² plating surface area to about 10,000 cells/mm² plating surface area, including about 1,850 cells/mm² surface area of the culture vessel or greater, or about 2,220 cells/mm² surface area of the culture vessel or greater, or about 2,590 cells/mm² surface area of the culture vessel or greater, or about 2,960 cells/mm² surface area of the culture vessel or greater, or about 2,220 cells/mm² surface area of the culture vessel or greater, or about 3,330 cells/mm² surface area of the culture vessel or greater, or about 3,703 cells/mm² surface area of the culture vessel surface area of the culture vessel or greater, or about 7,407 cells/mm² surface area of the culture vessel surface area of the culture vessel or greater.

In another example, cells are incubated under high density conditions e.g., until confluence or cell-to-cell contact is achieved, before detaching the cells. Such cells may be subsequently seeded at any density e.g., on a biocompatible matrix or in culture medium such as to promote their differentiation.

The optimum period of maintenance, culture or incubation in high density plating medium is determined empirically e.g., by calculating the optimum number of differentiated cells produced from aliquots of progenitor cells incubated at high density over a time course and subsequently incubated under conditions sufficient for differentiation to occur. Alternatively, or in addition, the optimum period of maintenance, culture or incubation in high density plating medium is determined empirically e.g., by determining fibroblast-specific and/or progenitor cell-specific marker expression by aliquots of progenitor cells incubated at high density over a time course. Alternatively, or in addition, the optimum period of maintenance, culture or incubation in high density plating medium is a period of time until adherence is achieved, i.e., a shorter time than required for cells to become adherent. Alternatively, or in addition, the optimum period of maintenance, culture or incubation in high density plating medium is up to about 5 days, including up to about 4 days or up to about 3 days or up to about 2 days or up to about 1 day i.e., up to about hours.

In another example of the invention, cells are introduced to high density culture conditions within about 6 hours to about 10 hours from their detachment, including within about 6 hours to about 9 hours from their detachment, or within about 6 hours to about 8 hours from their detachment, or within about 6 hours to about 7 hours from their detachment. In another example, cells are introduced to high density culture conditions within about 1 hour to about 6 hours from their detachment, including within about 5 hours to about 6 hours from their detachment, or within about 4 hours to about 5 hours from their detachment, or within about 3 hours to about 4 hours from their detachment, or within about 2 hours to about 3 hours from their detachment, or within about 1 hours to about 2 hours from their detachment. In another example, cells are introduced to high density culture conditions in less than about 5 hours from their detachment, including less than about 4 hours from their detachment, or less than about 3 hours from their detachment, or less than about 2 hours from their detachment, or less than about 1 hour from their detachment. In yet another example, the differentiated cells are incubated in a low-serum media, subjected to one or more means of achieving their detachment, and simultaneously introduced to high density culture conditions.

It will be apparent from the disclosure herein that similar or improved results e.g., in terms of numbers of progenitor cells capable of being differentiated into a plurality of different cell types and/or in terms of the degree of multipotency and/or pluripotency of the progenitor cells produced, are able to be produced by agonism of the Akt/(PKB) pathway and/or NF-kB pathway. Without being bound by any theory or mode of action, the inventor reasoned that such improvements are obtained by the incubation of differentiated cells in the presence of dexamethasone and/or one or more other specific modulators of RhoA and/or ROCK pathway and/or one or more specific modulators of SRC pathway, and optionally inducing cell detachment, and optionally incubating in low-serum media and/or and optionally maintaining cells at high density and/or optionally inducing the Akt/(PKB) and/or NF-kB pathway(s). The responses of cells to the combined incubation in the presence of dexamethasone and/or modulation of RhoA and/or ROCK pathway and/or modulation of SRC pathway with optional detachment of cells and/or optional low-serum incubation and/or optional maintenance, culture or incubation of cells at high density are likely to be a consequence of multiple pathways leading to progenitor cell formation.

Accordingly, in one example, the method of the present invention according to any example hereof further comprises incubating differentiated cells in a medium comprising an amount of an agonist or partial agonist of the Akt/(PKB) pathway and/or NF-kB pathway and for time sufficient to render the cells capable of being differentiated into a plurality of different cell types.

An alternative example of the present invention provides a method for producing a progenitor cell that is capable of being differentiated into a plurality of different cell types, said method comprising incubating differentiated cells in a medium comprising an amount of an agonist or partial agonist of the Akt/(PKB) pathway and/or NF-kB pathway and for time sufficient to render the cells capable of being differentiated into a plurality of different cell types.

Preferred agonists of the Akt/(PKB) pathway suitable for this purpose are described herein and include for example interleukin-1 (IL-1) and/or platelet derived growth factor (PGDF-BB) and/or insulin growth factor (IGF-1) and/or transforming growth factor-beta (TGF-β) and/or nerve growth factor (NGF) and/or carbachol or any active fragment or active chemical group thereof.

Preferred agonists of the NF-κB pathway suitable for this purposes are described herein and include e.g., tumor necrosis factor-alpha (TNF-α) and/or interleukin 1 (IL-1) or any active fragment thereof and/or lysophosphatidic acid (LPA) and/or lipopolysaccharide (LPS).

In one such example, the differentiated cells are incubated in a medium comprising an amount of an agonist or partial agonist of the Akt/(PKB) pathway and/or NF-kB pathway for a time and under conditions sufficient to agonise Akt/(PKB) pathway and/or NF-kB pathway but not sufficient to also modulate RhoA and/or ROCK pathway and/or SRC pathway in the cells. Alternatively, or in addition, the one or more specific modulators of RhoA and/or ROCK employed in the method of the invention according to any example hereof are different to the agonist or partial agonist of the Akt/(PKB) pathway and/or NF-kB pathway employed. Alternatively, or in addition, the one or more specific modulators of SRC employed in the method of the invention according to any example hereof are different to the agonist or partial agonist of the Akt/(PKB) pathway and/or NF-kB pathway employed.

In one example, the cells are incubated in the presence of an agonist of the Akt/(PKB) pathway and/or NF-κB pathway in media comprising dexamethasone and/or one or more modulator of RhoA and/or ROCK and/or one or more modulators of SRC as described herein and optionally in low-serum containing media or serum-free media and optionally further maintained, cultured or incubated at high density conditions to achieve optimum plasticity and/or multipotency or pluripotency. Such incubations are preferably for a time and under conditions sufficient to induce the Akt/(PKB) pathway and/or NF-κB pathway in the cells or a component thereof that is sufficient to render the cells capable of being differentiated into a plurality of different cell types.

In one example, the cells are further incubated in a medium comprising a dexamethasone and/or one or more other modulators of RhoA and/or ROCK pathway and/or one or more modulators of SRC pathway as described herein and optionally additionally incubated in or maintained on low-serum medium, and without supplementation of factors normally present in serum, e.g., before or during or following incubation in the presence of one or more agonists of the Akt/(PKB) pathway and/or one or more agonists of the NF-κB pathway. Such additional incubation is for at least about two days i.e., about 48 hours, and not exceeding about ten days i.e., about 240 hours, including for about two days or about three days or about four days or about five days or about six days or about seven days or about eight days or about nine days or about ten days. More preferably, the cells are incubated in low-serum media for a period between about five days and about nine days, including about five days or about six days or about seven days or about eight days or about nine days. Still more preferably, the cells are incubated in low-serum media for a period between about six days and about eight days, including about six days or about seven days or about eight days. In performing such examples, the agonist of the Akt/(PKB) pathway and/or NF-κB pathway may be added to the low-serum medium at any time point in the incubation period of at least about four days and not exceeding about ten days, or a shorter time as indicated herein. The skilled artisan is able to determine appropriate points for addition of one or more agonists to the medium without undue experimentation, and all such routine variations are within the scope of the present invention.

In another example, the cells are further detached by any means known in the art e.g., before, during or following incubation in the presence of one or more agonists of the Akt/(PKB) pathway and/or one or more agonists of the NF-κB pathway. Where low-serum medium is employed, is it preferred for such detachment to follow low-serum incubation. As will be apparent from the preceding disclosure, the cells may be detached by incubation in media containing a protease or a ligand of a protease activated receptor (PAR). Alternatively, the cells are detached by incubation in a Ca²⁺-free and Me-free media containing EDTA. Alternatively, the cells are detached by incubation in a medium containing citric saline.

In another example, the cells are further maintained, incubated or cultured under high density conditions e.g., after detachment and in a high density plating medium capable of supporting progenitor cells, or prior to detachment. Such incubation may be before, during or following incubation in the presence of one or more agonists of the Akt/(PKB) pathway and/or one or more agonists of the NF-κB pathway and/or before, during or following incubation in the presence of dexamethasone and/or one or more other modulators of RhoA and/or ROCK pathway and/or one or more modulators of SRC pathway.

In a another example, the cells are further incubated in low-serum medium and detached and wherein one or more agonists of the Akt/(PKB) pathway and/or one or more agonists of the NF-κB pathway is included during incubation with the low serum medium and/or in detachment medium. Alternatively, differentiated cells may be treated with one or more agonists of the Akt/(PKB) pathway and/or one or more agonists of the NF-κB pathway before commencing low-serum incubation and/or following detachment.

In a further example, the cells are incubated in low-serum medium, detached and maintained, incubated or cultured under high density conditions wherein one or more agonists of the Akt/(PKB) pathway and/or one or more agonists of the NF-κB pathway is included in low serum medium and/or in detachment medium and/or high density plating medium. Alternatively, differentiated cells may be treated with one or more agonists of the Akt/(PKB) pathway and/or one or more agonists of the NF-κB pathway before commencing low-serum incubation and/or following high density culture.

It is to be understood that the ordering of the incubation with dexamethasone and/or one or more other modulators of RhoA and/or ROCK pathway and/or one or more modulators of SRC pathway and optionally combined with protease or a ligand of a protease activated receptor (PAR), and optionally with the incubation in the presence of the agonist(s) of the Akt/(PKB) and/or NF-kB pathways, with or without extended incubation in low serum medium for about two days to about ten days or shorter periods, and with or without high density culture, maintenance or incubation conditions, and optionally incubation with one or more of one or more agonists of the Akt/(PKB) pathway and/or one or more agonists of the NF-κB pathway is not necessarily essential to the production of cells capable of undergoing subsequent differentiation into a plurality of different cell types. Conveniently, the differentiated cells are incubated in media comprising one or more agonists of the Akt/(PKB) pathway and/or one or more agonists of the NF-κB pathway before incubation with dexamethasone and/or one or more modulators of RhoA and/or ROCK and/or one or more modulators of SRC and optionally cell detachment and optionally additional maintenance, culture or incubation at high density conditions.

In one example, the invention according to any example hereof is performed on cultured cells ex vivo. In accordance with this example, it is preferred that the method comprises the first step of obtaining isolated cells from a suitable source e.g., from a commercial supplier. Alternatively, the cells have been isolated previously from a human or animal subject, including a syngeneic subject to whom progenitor cells produced by the method can optionally be administered e.g., topically, systemically, locally as an injectable and/or transplant and/or device, or in conjunction with one or more treatments for injuries, allografts or autografts. Alternatively, or in addition, an intermediate step of cell expansion in culture may be performed to increase the number of progenitor cells. Cell expansion in culture may be performed, for example, prior to administration of cells to a subject.

In accordance with this example, it is preferred for cells produced ex vivo to be formulated for use topically, systemically, or locally as an injectable and/or transplant and/or device, usually by adding necessary buffers. Alternatively, the cells are administered or formulated for use in conjunction with one or more treatments for injuries, allografts or autografts, to enhance wound repair and/or tissue regeneration.

In another example, the present invention provides a method for producing a progenitor cell that is capable of being differentiated into a plurality of different cell types, said method comprising incubating differentiated cells in situ with an amount of a modulator of SRC pathway and/or dexamethasone or any other modulator of RhoA and/or ROCK pathway and optionally with an amount of an agonist or partial agonist of the Akt/(PKB) pathway and/or NF-kB pathway and for time sufficient to render the cells capable of being differentiated into a plurality of different cell types. This example of the invention is performed on a human or animal subject in situ e.g., without obtaining isolated cells or performing an intermediate cell expansion in culture. In accordance with this example, dexamethasone and/or one or more modulators of RhoA and/or ROCK and/or one or more modulators of SRC and optionally one or more agonists or partial agonists of the Akt/(PKB) pathway and/or one or more agonists of the NF-kB pathway is(are) administered directly to a body site in the patient or in the vicinity of a body site in the patient in need of progenitor cells. Such tissue includes without limitation tissue in need of repair such as, for example, an injury, wound, burn, inflamed tissue, degenerated or damaged nerve, artery, muscle, bone, cartilage, fat, tendon, ligament, muscle or marrow stroma and combinations thereof.

In one example, the dexamethasone and/or the one or more other modulators of RhoA and/or ROCK pathway and/or the one or more modulators of SRC pathway and optionally the one or more agonists of the Akt/(PKB) pathway and/or the one or more agonists NF-κB pathway is(are) administered to the site in need thereof for a time and under conditions sufficient to achieve optimum plasticity and/or multipotency or pluripotency of differentiated cells at that site. Such incubation is preferably for a time and under conditions sufficient to modulate the SRC pathway and/or modulate the RhoA and/or ROCK pathway and/or induce the Akt/(PKB) pathway and/or induce the NF-κB pathway in the cells or a component thereof that is sufficient to render the cells capable of being differentiated into a plurality of different cell types. The skilled artisan is able to determine appropriate conditions for treatment with one or more modulators or agonists without undue experimentation, and all such routine variations are within the scope of the present invention. In one example, the method optionally further comprises administering a protease or a ligand of a protease activated receptor (PAR) to the site in need of progenitor cells. Without being bound by any theory or mode of action, the administration of a protease or PAR ligand promotes de-differentiation of differentiated cells and/or detachment of integrins from the extracellular matrix, thereby permitting the progenitor cells to enter circulation and regenerate damaged cells and tissues in situ.

It is to be understood that the ordering of the incubation with protease or a ligand of a protease activated receptor (PAR), and the incubation in the presence dexamethasone and/or the one or more other modulators of RhoA and/or ROCK pathway and/or the one or more modulators of SRC pathway and optionally the one or more agonists of the Akt/(PKB) pathway and/or the one or more agonists NF-κB pathway, is not necessarily essential to the production of cells at the body site capable of undergoing subsequent differentiation into a plurality of different cell types. Conveniently, dexamethasone and/or the one or more other modulators of RhoA and/or ROCK pathway and/or the one or more modulators of SRC pathway and optionally one or more agonist(s) of the Akt/(PKB) pathway and/or one or more agonist(s) of the NF-κB pathway are administered to the body site before administering a protease or a ligand of a protease activated receptor (PAR). In another example, one or more agonist(s) of the Akt/(PKB) pathway and/or one or more agonist(s) of the NF-κB pathway are administered to the body site before or at the same time with dexamethasone and/or the one or more other modulators of RhoA and/or ROCK and/or the one or more modulators of SRC.

Preferred differentiated cells on which the present invention is performed according to any example hereof include e.g., primary cells and immortalized cell lines. It is to be understood that the differentiated cells may also be terminally differentiated cells. The only requirement for such cells in performing this example of the invention is that they do not undergo apoptosis during the period of incubation in the presence of dexamethasone and/or the one or more other modulators of RhoA and/or ROCK pathway and/or the one or more modulators of SRC pathway and optionally in low serum media especially serum-free media and/or optionally during the period of incubation under high cell density conditions described herein. However, as indicated in the Examples, cells that normally undergo apoptosis during exposure to one or more modulators of SRC pathway and/or dexamethasone and/or one or more other modulators of RhoA and/or ROCK pathway and optionally during prolonged exposure to serum-free or low-serum media and optionally to high cell density culture, maintenance or incubation conditions can be used in other examples of the invention employing one or more agonists of the Akt/(PKB) pathway and/or NF-κB pathway to shorten the induction period required to induce plasticity in the starting cells and/or to enhance plasticity in the starting cells.

Cells of human origin, or potentially cells of porcine origin, are preferred for medical applications. More preferably the cells are derived from a tissue of a subject to whom a downstream product thereof is to be administered i.e., they are autologous. For veterinary or animal improvement purposes, the cells may be derived from any animal species in which they would be compatible when administered. Cells from any commercially-important animal species are contemplated herein e.g., pigs, cattle, horses, sheep, goats, dogs, cats, etc. As with human applications, it is preferred to use autologous cells for such applications to minimize rejection. Exemplary differentiated cells for use in the present invention include skin cells and/or epidermal cells and/or fibroblasts and/or keratinocytes and/or melanocytes and/or epithelial cells and/or neural cells such as those derived from the peripheral nervous system (PNS) and central nervous system (CNS) and/or glial cells and/or Schwann cells and/or astrocytes and/or oligodendrocytes and/or microglial cells and/or lymphocytes and/or T cells and/or B cells and/or macrophages and/or monocytes and/or dendritic cells and/or Langerhans cells and/or eosinophils and/or adipocytes and/or cardiac muscle cells and/or osteoclasts and/or osteoblasts and/or endocrine cells and/or β-islet cells and/or endothelial cells and/or granulocytes and/or hair cells and/or mast cells and/or myoblasts and/or Sertoli cells and/or striated muscle cells and/or zymogenic cells and/or oxynitic cells and/or brush-border cells and/or goblet cells and/or hepatocytes and/or Kupffer cells and/or stratified squamous cells and/or pneumocytes and/or parietal cells and/or podocytes and/or synovial cells and/or serosal cells and/or pericytes and/or osteocytes and/or Purkinje fiber cells and/or myoepithelial cells and/or megakaryocytes.

Chondrocytes can also be used in the present invention for those examples that specifically require incubation with dexamethasone or one or more other modulators of RhoA and/or ROCK pathway and/or one or more modulators of SRC pathway.

In a particularly preferred example, the cells are fibroblasts, preferably of dermal origin.

It will be apparent from the disclosure herein that, in performing the present invention, the differentiated cell is not merely reprogrammed from one developmental pathway to another developmental pathway, but that a progenitor cell is produced that can be stored or otherwise maintained until required for downstream processing e.g., to give rise to different cell types. Without compromising the generality of the present invention, the method of the invention thus produces cells having one or more stem-cell like attributes in so far as they are multipotent, pluripotent or totipotent progenitor cells. For example, the cells produced in accordance with the invention are a novel population of stem cells e.g., having undetectable or low (negligible) levels of at least one and preferably a plurality of the following cell markers as determined by standard cell marker detection assays: CD90, CD117, CD34, CD113, FLK-1, tie-2, Oct 4, GATA-4, NKx2.5, Rex-1, CD105, CD117, CD133, MHC class I receptor and MHC class II receptor. By the term “standard cell marker detection assay” is meant a conventional immunological or molecular assay formatted to detect and optionally quantify one of the foregoing cell markers (i.e., CD90, CD117, CD34 etc.). Examples of such conventional immunological assays include Western blotting, ELISA, and RIA. Preferred antibodies for use in such assays are provided below. See generally, Harlow and Lane in Antibodies: A Laboratory Manual, CSH Publications, N.Y. (1988), for disclosure relating to these and other suitable assays. Particular molecular assays suitable for such use include polymerase chain reaction (PCR) type assays using oligonucleotide primers e.g., as described in WO 92/07075 and/or Sambrook et al. in Molecular Cloning: A Laboratory Manual (2d ed. 1989) and/or Ausubel et al., Current Protocols in Molecular Biology, John Wiley & Sons, New York, 1989.

Accordingly, in a further example, the present invention provides a method for producing a progenitor cell that is capable of being differentiated into a plurality of different cell types, said method comprising incubating differentiated cells in a medium comprising a modulator of SRC pathway and/or dexamethasone or any other modulator of RhoA and/or ROCK pathway, and detaching the cells for example by incubating the cells in media comprising a protease or a ligand of a protease activated receptor (PAR) and optionally further incubating differentiated cells in a low serum concentration and without supplementation of factors normally present in serum, and/or optionally further incubating the cells under high density conditions, preferably in a medium capable of supporting progenitor cells to thereby render the cells capable of being differentiated into a plurality of different cells types, and maintaining or storing the cells as progenitor cells.

In a further example, the present invention provides a method for producing a progenitor cell that is capable of being differentiated into a plurality of different cell types, said method comprising incubating differentiated cells in a culture medium comprising dexamethasone and/or a culture medium comprising one or more other modulators of RhoA and/or ROCK pathway and/or a culture medium comprising one or more modulators of SRC pathway, and optionally in a culture a medium comprising an amount of an agonist or partial agonist of the Akt/(PKB) pathway and/or NF-κB pathway, and optionally incubating the cells in media comprising a protease or a ligand of a protease activated receptor (PAR), and optionally further incubating differentiated cells in a low serum concentration and without supplementation of factors normally present in serum, and optionally further incubating the cells under high density conditions, preferably in a medium capable of supporting progenitor cells and for time sufficient to render the cells capable of being differentiated into a plurality of different cell types, and maintaining or storing the cells as progenitor cells.

Preferably, the method according to any example hereof further comprises providing the differentiated cells e.g., as an adherent cell culture.

In a further example, the present invention further comprises genetically engineering the progenitor cells to express a protein of interest, such as for example, a macromolecule necessary for cell growth and/or morphogenesis and/or differentiation and/or tissue building and combinations thereof, and preferably, a bone morphogenic protein and/or a bone morphogenic-like protein and/or an epidermal growth factor and/or a fibroblast growth factor and/or a platelet derived growth factor and/or an insulin like growth factor and/or a transforming growth factor and/or a vascular endothelial growth factor and/or Ang-1 and/or PIGF and combinations thereof.

In a further example, the present invention encompasses a cell culture comprising progenitor cells produced by the method disclosed according to any example hereof. Preferably, the cell culture is for treatment of the human or animal body by therapy or prophylaxis. In one example, the cell culture comprising progenitor cells produced by the method disclosed according to any example hereof is for the treatment or prophylaxis of cancer.

It will also be apparent from the disclosure herein that the stem cell-like attribute of the progenitor cells produced in accordance with the inventive method confer the ability to produce one or more cells or tissues from them in medical and veterinary applicants and for animal improvement. In this respect, methods for producing such different cell types from a unipotent, multipotent, pluripotent or totipotent progenitor cells are known in the art and/or described herein.

Accordingly, a further example of the present invention provides a process for producing a differentiated cell said process comprising producing a progenitor cell according to any example of the invention hereof and then incubating the progenitor cell for a time and under conditions sufficient to induce differentiation of the progenitor cell into a differentiated cell.

In one such example, the method further comprises incubating the progenitor cell in the presence of at least one mitogen for a time and under conditions sufficient to promote or enhance cell replication and/or cell division of the progenitor cell e.g., by stimulating mitosis, thereby inducing differentiation of the progenitor cell into a differentiated cell. The mitogens falling within the scope of the invention according to any example hereof include but are not limited to Fibroblast growth factors such as FGF-2, amphiregulin, EGF, Sonic hedgehog (Shh), Engrailed 1 (En1) and/or Engrailed 2 (En2), phytohemagglutinin (PHA) including PHA-P, -M, -W, -C, -L and -E+L, pokeweed mitogen (PWM), Concanavalin A (Con-A), lipopolysaccharide (LPS), wheat germ agglutinin (WGA) and soybean agglutinin (SBA), or other genes, proteins and the like.

In another example, the method further comprises incubating the progenitor cell in the presence of at least one morphogen for a time and under conditions sufficient to provide biological signalling suitable for promoting or enhancing cellular specialization and/or cellular differentiation of the progenitor cell thereby inducing differentiation of the progenitor cell into a differentiated cell. The morphogens falling within the scope of the invention according to any example hereof include but are not limited to retinoic acid and/or homeodomain transcription factors such as Dlx transcription factors eg., Dlx5 and/or fibroblast growth factors such as FGF10, FGF8, FGF4 and/or fibroblast growth factor receptors such as FGFR1, FGFR2 and/or a T-box transcription factors such as TBX4, TBX5 and/or members of the wingless-type MMTV integration site (WNT) signalling factors such as WNT2B, WNT8C, WNT7A, WNT5A, WNT3A and/or Sonic hedgehog (Shh) and/or Bone morphogenetic protein 2 (BMP2) and/or Radical fringe (Rfng) and/or Notch signalling molecules such as Notch, Notch 1, Notch 2, Notch 3, Notch 4 and/or a Notch ligand such as Notch Receptor S1, Jagged 1 (JAG1), Jagged 2 (JAG2) and/or a modulator of Notch signaling such as Lunatic fringe (Lfng), and/or homeodomain proteins such as Lmx1 and/or Ser2 and/or Engrailed 1 (En1) and/or Engrailed 2 (En2) or other genes, proteins and the like.

The progenitor cells may be used to produce any cell type. For example, the differentiated cell produced by the process may be cardiac tissue cells and/or a skin cell and/or epidermal cell and/or keratinocyte and/or melanocyte and/or epithelial cell and/or neural cell and/or dopaminogenic cell and/or glial cell and/or Schwann cell and/or astrocyte and/or oligodendrocyte and/or microglial cell and/or blood cell and/or lymphocyte and/or T cell and/or B cell and/or macrophage and/or monocyte and/or dendritic cell and/or lagerhans cell and/or eosinophil and/or adipocyte and/or cardiomyocyte and/or cardiac muscle cell and/or cardiac fibroblast and/or osteoclast and/or osteoblast and/or endocrine cell and/or β-islet cell and/or insulin secreting cell and/or endothelial cell and/or epithelial cell and/or granulocyte and/or hair cell and/or mast cell and/or myoblast and/or Sertoli cell and/or striated muscle cell and/or zymogenic cell and/or oxynitic cell and/or brush-border cell and/or goblet cell and/or hepatocyte and/or Kupffer cell and/or stratified squamous cell and/or pneumocyte and/or parietal cell and/or podocyte and/or synovial cell and/or serosal cell and/or pericyte and/or chondrocyte and/or osteocyte and/or Purkinje fiber cell and/or myoepithelial cell and/or megakaryocyte, etc. In one preferred example, the differentiated cell produced by the method according to any example hereof is cardiac tissue cell, such as cardiac fibroblast.

As with the progenitor cells of the invention, such differentiated cells can be maintained by one or a combination of strategies including those involving maintenance in vitro. The differentiated cells can be maintained by strategies including those involving maintenance ex vivo and/or in vivo.

Accordingly, in a further example, the present invention encompasses a cell culture comprising differentiated cell produced from a progenitor cell in accordance with the process disclosed according to any example hereof. Preferably, the cell culture is for treatment of the human or animal body by therapy or prophylaxis. In one example, the cell culture is for treatment or prophylaxis of cancer. In another example, the cell culture is for treatment or prophylaxis of tissue organ damage including but not limited to cardiac injury such as myocardial infraction.

In another example the progenitor cells may used to differentiate into tissues and/or organs. According to this example, the progenitor cells produced by the method of the invention and/or the differentiated cells derived therefrom may be used for regenerating and/or building any tissue or organ. For example, the regenerated or built tissue produced by the process includes a cardiac tissue and/or a cardiac muscle tissue and/or a cardiomyocyte tissue and/or a cardiac fibroblast tissue and/or a skin tissue and/or an epidermal tissue and/or a keratinocyte tissue and/or a melanocyte tissue and/or an epithelial and/or a dermal dendrocyte tissue and/or a nervous tissue and/or a muscle tissue and/or a connective tissue and/or a mucosal tissue and/or a cardiac tissue and/or a cardiac muscle tissue and/or a cardiomyocyte tissue and/or a cardiac fibroblast tissue and/or an endocrine tissue and/or an adipose tissue and/or a galial tissue and/or a collagen or fibrin tissue and/or an osseous or bone tissue and/or an osteocyte tissue and/or a blood vessel tissue e.g., an endothelial tissue and/or a lymphoid tissue and/or an endocrine tissue e.g., a pancreatic endocrine tissue and/or an islet tissue e.g., β-islet tissue and/or a chondrocyte tissue and/or a hepatic tissue and/or a eosinophil tissue and/or an osteoblast tissue and/or an osteoclast tissue and/or a hair tissue and/or a bone marrow tissue and/or a striated muscle tissue and/or a reproductive tissue and/or a synovial tissue, etc. For example, the organ regenerated or produced by the process includes heart and/or artery and/or trachea and/or skin and/or hair and/or liver and/or spleen and/or heart and/or kidney and/or muscle and/or bone and/or a limb such as a finger and/or toe and/or arm and/or leg and/or nose and/or ear and/or panaceas and/or lung and/or lympoid organ and/or female reproductive organ e.g., ovary, uterus, vagina, cervix, and/or fallopian tubes and/or male reproductive organ e.g., testis and/or nerve and/or blood vessel and/or small intestine and/or large intestine and/or endocrine organ or hormone-secreting gland e.g., pituitary gland and/or bladder and/or dental tissues such as teeth, or dentin, etc.

Accordingly in a one example, the present invention further provides a method of regenerating, repairing and/or building a tissue and/or an organ, said method comprising culturing or perfusing the progenitor cells produced according to any example hereof and/or culturing differentiated cells derived from said progenitor cells on or into a biocompatible scaffolding material or matrix. In one example, the scaffold material or matrix, provides the mitogens and/or morphogens and/or biological signalling suitable for promoting or enhancing replication and/or differentiation of the progenitor cells and/or tissue building repair or regeneration and/or organ building, repair or regeneration. In another example, the scaffold material or matrix provides the structure or outline to a tissue to be repaired, regenerated or built, and/or organ to be repaired, regenerated or built. Such scaffold material or matrix may include for example a non-cellular matrix comprising proteoglycan and/or collagen or other suitable material for tissue building or organ building processes to occur. In one example, the scaffold material or matrix comprises synthetic or semi-synthetic fibers such as Dacron™, Teflon™ or Gore-Tex™. In another example, the scaffold material or matrix comprises a decellularized organ or tissue stripped of its cells by any means known in the art.

Alternatively, or in addition, the present invention further provides a method of regenerating, repairing and/or building a tissue and/or an organ, said method comprising providing the progenitor cells an agent selected from a neuropeptide Y (NPY) and/or a fragment of neuropeptide Y and/or a variant of neuropeptide Y and/or a compound capable of inducing expression of a gene encoding a neuropeptide Y protein or fragment or variant thereof and/or a cell that produces a neuropeptide Y and/or an agonist or antagonist of a neuropeptide Y receptor and combinations thereof, wherein said agent induces regeneration, repair or building of a tissue or organ. In one preferred example, the progenitor cells are provided to a site of injury in a tissue and/or organ to induces regeneration, repair or building of a tissue or organ at the site of injury. In one preferred example, the agent in provided to the progenitor cells at the site of injury

Alternatively, or in addition, the present invention provides a method of regenerating, repairing and/or building a tissue and/or an organ, said method comprising providing the progenitor cells an agent selected from a neuregulin and/or a fragment of a neuregulin and/or a compound capable of inducing expression of a neuregulin gene and/or an agonist or antagonist of a receptor for neuregulin and any combination thereof, wherein said agent induces regeneration, repair or building of a tissue or organ. In one preferred example, the progenitor cells are provided to a site of injury in a tissue and/or organ to induces regeneration, repair or building of a tissue or organ at the site of injury. In one preferred example, the agent in provided to the progenitor cells at the site of injury

Alternatively, or in addition, the present invention provides a method of regenerating, repairing and/or building a tissue and/or an organ, said method comprising providing the progenitor cells an agent selected from a neurotrophin and/or a fragment of a neurotrophin and/or a compound capable of inducing expression of a neurotrophin gene and/or an agonist or antagonist of a receptor for a neurotrophin and any combination thereof, wherein said agent induces regeneration, repair or building of a tissue or organ. In one preferred example, the progenitor cells are provided to a site of injury in a tissue and/or organ to induces regeneration, repair or building of a tissue or organ at the site of injury. In one preferred example, the progenitor cells are provided to a site of injury in a tissue and/or organ to induces regeneration, repair or building of a tissue or organ at the site of injury. In one example, the agent in provided to the progenitor cells at the site of injury. In one non-limiting example, the neurotrophin is nerve growth factor (NGF), or neurotrophic factor 3 (NT-3), or brain derived neurotrophic factor (BDNF), or neurotrophic factor 4 (NT-4), neurotrophic factor 5 (NT-5) or Ciliary Neurotrophic Factor CNTF. In a particularly preferred example, the neurotrophin is NGF.

In one example, tissue and/or organ regeneration, repair or building occurs in vitro externally of the body of an organism, including a human or other mammalian subject in need thereof. In another example, the tissue and/or organ regeneration, repair or building occurs in vivo in an organism, including a human or other mammalian subject in need thereof.

In one example, tissue regeneration or repair or building is used to reduce or eliminate scar tissue.

In one example, the present invention conveniently utilizes a starter cell, i.e., any differentiated primary cell, cell strain, or cell line that is derived and/or obtained from the same tissue type and/or organ type as the tissue and/or organ which is being regenerated, repaired and/or built. For example, skin fibroblasts from a limb or an appendage are used to produce progenitor cells that are subsequently regenerated into a limb or an appendage e.g., a finger, a toe, an arm or a leg. In another example, cardiac fibroblasts such as from a heart or artery are used to produce progenitor cells that are subsequently regenerated into a limb or a cardiovascular organ such as heart or artery.

A further example of the present invention provides for the use of a progenitor cell produced according to any example hereof or a differentiated cell or tissue or organ derived there from in the prophylactic or therapeutic treatment of the human or animal body. In one example, the use of a progenitor cell produced according to any example hereof or a differentiated cell or tissue or organ derived there from is in the treatment or prophylaxis of cancer. In one example, the use of a progenitor cell produced according to any example hereof or a differentiated cell or tissue or organ derived there from is in the treatment or prophylaxis of cardiac or cardiovascular damage or cardiac failure.

In a further example, the present invention provides for the use of a progenitor cell produced according to any example hereof or a differentiated cell or tissue or organ derived there from in the preparation of a cell preparation for the prophylactic or therapeutic treatment of a condition in a subject alleviated by administering stem cells or tissue derived from stem cells to a subject or by grafting stem cells or tissue derived from stem cells into a subject or by transplanting stem cells or tissue derived from stem cells into a subject. In one example, the condition alleviated by administering, grafting or transplanting stem cells or tissue derived from stem cells to a subject is cancer. In another example, the condition alleviated by administering, grafting or transplanting stem cells or tissue derived from stem cells to a subject is cardiac tissue damage or cardiovascular damage.

In a further example, the present invention provides for the use of an isolated, non-culture progenitor cell in the preparation of a medicament for administration to a subject, wherein the non-culture progenitor cell is obtained via a method of the invention according to any example hereof. By “non-culture progenitor cell” is meant a progenitor cell of the present invention produced without cell expansion in vitro and preferably used within about twenty four hours following their preparation by a method described herein according to any example.

In a further example, the present invention provides for the use of an isolated, non-culture progenitor cell in the preparation of a medicament for stimulating or enhancing tissue repair in a subject, wherein the non-culture progenitor cell is obtained via a method of the invention according to any example hereof.

In a further example, the present invention provides for the use of an isolated, non-culture progenitor cell in the preparation of a medicament for stimulating or enhancing tissue formation in a subject, wherein the non-culture progenitor cell is obtained via a method of the invention according to any example hereof.

Preferably, the differentiated cells, tissues or organs are introduced to the human or animal body by grafting means, and it is clearly within the scope of the present invention to provide a graft that includes isolated progenitor cells or differentiated cells or tissues or organs derived therefrom.

As used herein, the term “graft” shall be taken to mean a cell or tissue or organ preparation that includes an isolated progenitor cell produced in accordance with any example of the invention hereof and/or a differentiated cell, tissue or organ derived in vitro or in vivo from said isolated progenitor cell and, optionally comprising one or more other cells and/or mitogens and/or morphogens and/or a matrix suitable for promoting or enhancing differentiation and/or tissue building, repair or regeneration and/or organ building, repair or regeneration. For example, a “graft” includes tissue or organ that is produced by culturing progenitor cells of the invention and/or differentiated cells derived from said progenitor cells onto a matrix e.g., a non-cellular matrix comprising proteoglycan and/or collagen or other suitable material for tissue building or organ building processes to occur e.g., synthetic or semi synthetic fibers that give structure to a graft, such as Dacron™, Teflon™ or Gore-Tex™. By “graft” is also meant progenitor cells of the invention that have been administered to a recipient and become part of one or more tissues or organs of that recipient. A graft of the invention may also take the form of a tissue preparation or tissue culture preparation in which progenitor cells of the invention have been combined with other cells and/or one or more growth factors and/or one or more mitogens to promote cell proliferation and/or one or more morphogens to promote differentiation and/or cell specialization that produce an intended graft. If desired, the preparation can be combined with synthetic or semi synthetic fibers to give structure to the graft. Fibers such as Dacron™, Teflon™ or Gore-Tex™ are preferred for certain applications. Sometimes the word “engraftment” will be used to denote intended assimilation of the progenitor cells or derivative differentiated cells tissue or organs into a target tissue, organ or organism, including a human or other mammalian subject. Preferred engraftment involves neural tissue, cardiovascular tissue, cardiac tissue, splenic tissue, pancreatic tissue, etc.

In using the cells of the invention for medical applications or veterinary applications, or in animal improvement, immunological relationship between a donor of the differentiated cells used to produce the progenitor cells, and the recipient of the progenitor cells or a cell or tissue or organ derived from the progenitor cells, can be allogenic, autologous, or xenogeneic as needed. In one example, the donor and recipient will be genetically identical and usually will be the same individual (syngeneic). In this instance, the graft will be syngeneic with respect to the donor and recipient. In one example, the progenitor cells and/or graft will be immune tolerated in the recipient subject.

A further example of the present invention provides a method for preventing, treating or reducing the severity of a disease or disorder in a human or animal subject said method comprising administering to the human or animal subject in need of treatment at least one isolated progenitor cell or graft or a combination thereof. Preferably, the administration is sufficient to prevent, treat or reduce the severity of the disease or disorder in the human or animal subject.

In one example, the method further includes incubating the cells or graft in the human or animal subject for at least about a week, preferably between from about two to eight weeks. It will be apparent to those working in the field that the incubation period is flexible and can be extended or shorten to address a particular indication or with respect to the health or age of the individual in need of treatment. Typical amounts of progenitor cells to use will depend on these and other recognized parameters including the disease to be treated and the speed of recovery needed. However for most applications between from about 1×10³ to about 1×10⁷ progenitor cells per grafting site will suffice, typically about 1×10⁵ of such cells. Cells may be administered by any acceptable route including suspending the cells in saline and administering same with a needle, stent, catheter or like device. In examples in which myocardial ischemia or an infarct is to be addressed, the administration will be a bolus injection near or directly into the desired site.

In another example, the method further includes administering to the human or animal subject in need of treatment at least one growth factor and/or at least one mitogen and/or at least one morphogen and/or a functional fragment thereof to promote tissue regeneration and/or cellular proliferation. Alternatively, or in addition, the method can include administering to the mammal at least one nucleic acid encoding at least one growth factor and/or at least one mitogen and/or at least one morphogen and/or a functional fragment thereof. For example, methods for administering such nucleic acids to mammals have been disclosed by U.S. Pat. No. 5,980,887 and WO 99/45775.

In yet another example, the method further includes administering to the human or animal subject one or more other progenitor cells.

Further provided by the invention is a pharmaceutical composition for preventing, treating or reducing the severity of a disease or disorder, said composition comprises a population of progenitor cells or graft produced according to any example hereof and a pharmaceutically acceptable carrier. Optionally, the composition comprises directions for preparing, maintaining and/or using the progenitor cells or graft, including any cell culture, tissue or organ. In one example, the product further includes at least one growth factor and/or mitogen and/or morphogen and/or a functional fragment thereof. In another example, the product further comprises at least one nucleic acid encoding a growth factor, mitogen, morphogen and/or a functional fragment thereof.

Further provided by the invention is a kit for building, repairing or regenerating a tissue or an organ, said kit comprises a population of progenitor cells produced according to any example hereof and a scaffold or matrix for culturing the progenitor cells or differentiated cells produced from said progenitor cells. Optionally, the composition comprises directions for preparing, maintaining and/or using the progenitor cells or graft, including any cell culture, tissue or organ. In one example, the product further includes at least one growth factor and/or at least one mitogen and/or at least one morphogen and/or a functional fragment thereof. In another example, the product further comprises at least one nucleic acid encoding a growth factor, and/or a mitogen, and/or a morphogen and/or a functional fragment thereof.

Further provided by the invention are isolated differentiated cell derived from progenitor cells produced according to any example hereof. Also provided by the invention is a scaffold or matrix comprising progenitor cells or one or more populations of differentiated cells derived from the progenitor cells as described according to any example hereof. Further provided by the invention is any tissue or any organ derived in vitro or in vivo from isolated progenitor cells produced according to any example hereof or any tissue or any organ derived in vitro or in vivo from differentiated cells derived from the progenitor cells as described according to any example hereof.

Unless the context requires otherwise or specifically stated to the contrary, integers, steps, or elements of the invention recited herein as singular integers, steps or elements clearly encompass both singular and plural forms of the recited integers, steps or elements.

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, developmental biology, mammalian cell culture, recombinant DNA technology, histochemistry and immunohistochemistry and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R.W. Masters, ed., 2000), ISBN 0199637970, whole of text;
6. Methods In Enzymology (S. Colowick and N. Kaplan, eds., Academic Press, Inc.), whole of series;

### DETAILED DESCRIPTION OF THE PREFERRED EMBODIMENTS

### 1. Cell Types-Starting Material

The present invention provides that any differentiated animal cell type including terminally differentiated animal cells may be used as starting cells for the method of the invention. For example, the starting cells are primary cells, a cell strain, or a cell line.

By “differentiated cell type” is meant a differentiated animal cell type that expresses defined specialized properties that are characteristic of that cell type. These defined specialized properties are passed onto daughter cells if the differentiated cell type undergoes cellular division. The differentiated cell type may be a cell type that is not actively proliferating. Methods for determining the expressed specialized properties will be apparent to the skilled artisan and/or described herein.

Preferably, the starting cells are readily available in substantial quantities. The starting cells may be derived from any animal and preferably from a mature adult animal. The type of animal preferably includes but is not limited to humans, and includes any animal species such as: other primate such as ape and/or chimpanzee and/or gorilla and/or monkey and/or or orangutan and/or horse and/or cow and/or goat and/or sheep and/or pig and/or dog and/or cat and/or bird and/or fish and/or rabbit and/or rodent such as a mouse or rat.

The present invention also provides that the starting cells may be expanded in cell culture prior to use. Methods for expanding the starting cells in culture will be apparent to the skilled artisan and/or described herein.

### 1.1 Primary Cell Cultures, Cell Strains, and Cell-Lines

Methods for obtaining primary cultures of differentiated starting cells and others are well known in the art, and usually include obtaining the tissue from a biopsy and/or amputated limb and/or secretion and/or excretion and/or other source. The tissue may be derived from any part of the body that is readily available including, but not limited to organs such as skin, bone, gut, pancreas, thymus, spleen, blood, bone marrow, spine, or any nervous tissue.

In a preferred example, the tissue is derived from an adult donor. In a further preferred example the tissue is derived from a patient, since this facilitates autologous transplants and thus reduces the likelihood of adverse immunogenic reactions in the patient. In another preferred example, the tissue is derived from a damaged and/or amputated organ e.g., limb or appendage which is in need of regeneration, repair or replacement in the patient.

The tissue sample comprising the desired differentiated starting cells may also contain connective tissue, for example a skin biopsy. As a non-limiting example, in order to isolate human dermal fibroblasts derived from a mammal, preferably a human, a skin biopsy is obtained which is then minced or otherwise cut into smaller pieces or treated to release the differentiated cell. For instance, and without limiting the invention to any particular method of obtaining a cell to be used in the methods described herein, the tissue is often treated with a collagenase or other protease in order to disassociate the cells from the tissue aggregate. These cells are then placed in a tissue culture flask, or dish, along with a nutrient tissue culture media and propagated at a suitable temperature and a suitable CO₂ saturation. The suitable temperature is often from about 35° C. to about 37° C., and the suitable CO₂ saturation is often about 5-10% in air.

Optionally, the tissue sample may also be a suspension containing cells, or comprising a liquid such as a blood sample, or an aspirate such as fluid obtained from the spinal column, or from bone marrow. Samples obtained in suspension and/or liquid form are further processed by centrifugation, or separation, and culture techniques. Blood cells and lymphocytes are often obtained from whole blood treated with heparin or another anti-coagulant. The blood is centrifuged on a gradient, such as a Ficoll gradient, and the lymphocytes and other blood cells form a distinct layer often referred to as the “buffy coat”. Primary lymphocytes procured by this method can be further separated by their adherence to glass or plastic (monocytes and macrophages adhere, other lymphocytes, in general, do not adhere). Methods for obtaining and culturing both solid tissue and blood cells from a human are well known in the art and are described in, for example, Freshney (2000, Culture of Animal Cells: A Manual of Basic Techniques, 4th Edition, Wiley-Liss, New York, N.Y.).

As a further non-limiting example, peripheral nerve tissue can be obtained using surgical procedures such as nerve biopsies, amputated limbs, and from organ donors and by any other methods well known in the art or to be developed. Potential sources of peripheral nerve include the sciatic nerve, cauda equina, sural nerve of the ankle, the saphenous nerve, the sciatic nerve, or the brachial or antebrachial nerve of the upper limb.

A preferred amount for the starting nerve tissue is between about 10 milligrams to about 10 grams, preferably between about 100 milligrams to about 1-2 gram. Primary human Schwann cells can be isolated and cultured using the methods detailed elsewhere in this invention or methods known in the art. Other methods for the isolation and culture of Schwann cells and other neural cells are well known in the art, and can readily be employed by the skilled artisan, including methods to be developed in the future. The present invention is in no way limited to these or any other methods, of obtaining a cell of interest.

The skilled artisan would appreciate, based upon the disclosure provided herein, that the particular method for obtaining a differentiated starting cell of interest is not limited in any way, but encompasses methods for isolating a cell of interest well known in the art or to be developed in the future.

Preferably, the differentiated starting cells employed in the present invention include, but are not limited to skin cells and/or epidermal cells such as fibroblasts and/or keratinocytes and/or melanocytes and/or epithelial cells and the like and/or cardiac tissue and/or cardio vascular tissue cells such as cardiac muscle cells, cardiac fibroblasts, cardiomyocytes and/or neural cells such as those derived from the peripheral nervous system (PNS) and central nervous system (CNS) including, but not limited to, glial cells, such as, e.g., Schwann cells and/or astrocytes and/or oligodendrocytes and/or microglial cells and/or and blood cells, such as lymphocytes, including T cells and/or B cells and/or macrophages and/or monocytes and/or dendritic cells and/or Lagerhans cells and/or eosinophils and the like and/or adipocytes and/or cardiac muscle cells and/or cardiac fibroblasts and/or osteoclasts and/or osteoblasts and/or endocrine cells and/or β-islet cells of the pancreas and/or endothelial cells and/or epithelial cells and/or granulocytes and/or hair cells and/or mast cells and/or myoblasts and/or Sertoli cells and/or striated muscle cells and/or zymogenic cells and/or oxynitic cells and/or brush-border cells and/or goblet cells and/or hepatocytes and/or Kupffer cells and/or stratified squamous cells and/or pneumocytes and/or parietal cells and/or podocytes and/or synovial cells and/or such as synovial fibroblasts and/or serosal cells and/or pericytes and/or chondrocytes and/or osteocytes and/or Purkinje fiber cells and/or myoepithelial cells and/or megakaryocytes, and the like.

The present invention further includes starting cells of primary cells from any of the aforementioned sources that may be purchased from any commercial source including PromoCell® (Banksia Scientific Company, QLD).

The present invention further includes starting cells of primary cells obtained from or present in the human or animal body.

The present invention further includes starting cells of primary strains or cells lines established in culture, or to be established in culture in the future that may be purchased from any commercial source including American Type Culture Collection (Rockville, Md.).

By “primary strain” shall be taken to indicate any cell type derived as described by any example herein that is established in culture, and that expresses defined specialized properties that are passed onto daughter cells during cellular division, and have a limited life span in culture. Methods for determining the expressed specialized properties will be apparent to the skilled artisan and/or described herein.

By “cell line” shall be taken to indicate any cell type derived as described by any example herein that is established in culture, and that expresses defined specialized properties that are passed onto daughter cells during cellular division, and have an indefinite life span in culture. Methods for determining the expressed specialized properties will be apparent to the skilled artisan and/or described herein.

Preferably, the primary strain, or cell line used as starting material has specialized properties that define the cell type, and such defined cell types include, but are not limited to: skin cells and/or epidermal cells and/or such as fibroblasts and/or keratinocytes and/or melanocytes and/or epithelial cells and/or cardiac tissue or cardio vascular tissue cells such as cardiac muscle cells, cardiac fibroblasts, cardiomyocytes, and/or neural cells such as those derived from the peripheral nervous system (PNS) and central nervous system (CNS) including, but not limited to, glial cells, such as, e.g., Schwann cells and/or astrocytes and/or oligodendrocytes and/or microglial cells and/or blood cells, such as lymphocytes, including T cells and B cells, macrophages and/or monocytes and/or dendritic cells and/or Lagerhans cells and/or eosinophils and the like and/or adipocytes and/or osteoclasts and/or osteoblasts and/or endocrine cells and/or β-islet cells of the pancreas and/or endothelial cells and/or epithelial cells and/or granulocytes and/or hair cells and/or mast cells and/or myoblasts and/or Sertoli cells and/or striated muscle cells and/or zymogenic cells and/or oxynitic cells and/or brush-border cells and/or goblet cells and/or hepatocytes and/or Kupffer cells and/or stratified squamous cells and/or pneumocytes and/or parietal cells and/or podocytes and/or synovial cells and/or such as synovial fibroblasts and/or serosal cells and/or pericytes and/or chondrocytes and/or osteocytes and/or Purkinje fiber cells and/or myoepithelial cells and/or megakaryocytes, and the like.

In one example, the present invention utilizes a starter cell that is not sensitive to incubation with dexamethasone used by the method of the invention. In another example, the present invention utilizes a starter cell that is not sensitive to incubation with one or more modulators of RhoA and/or ROCK pathway. In another example, the present invention utilizes a starter cell that is not sensitive to incubation with one or more modulators of SRC pathway. For example, such a starter cell is not sensitive to culturing with dexamethasone and/or one or more other modulators of RhoA and/or ROCK pathway and/or one or more modulators of SRC pathway for the period the cell is required to be cultured, maintained or incubated in the presence of dexamethasone or the one or modulators.

By “starter cell” is taken to mean any differentiated primary cell, cell strain, or cell line as derived and/or obtained by any example described herein.

By “not sensitive to incubation with dexamethasone and/or with one or more modulators of RhoA and/or ROCK pathway” or “not sensitive to modulation of RhoA and/or ROCK pathway” is taken to mean that the cell does not undergo cell death and/or has activated one or more pro-survival pathway(s). For example, cell death is the result of the induction or outcome of any cellular process that includes but is not limited to necrosis, apoptosis or programmed cell death.

By “not sensitive to incubation with one or more modulators of SRC pathway” or “not sensitive to modulation of SRC pathway” is taken to mean that the cell does not undergo cell death and/or has activated one or more pro-survival pathway(s). For example, cell death is the result of the induction or outcome of any cellular process that includes but is not limited to necrosis, apoptosis or programmed cell death.

By “pro-survival pathway” is taken to mean any pathway that overcomes the induction of one or more cellular processes that result in cell death.

In another example, the present invention utilizes a starter cell that is not sensitive to incubation with dexamethasone in the cell, and that is induced in one or more pro-survival pathway(s) such that incubation time with dexamethasone in the cell can be reduced and/or does not significantly affect viability of the cell. Alternatively, or in addition, the present invention utilizes a starter cell that is not sensitive to incubation with one or more modulators of RhoA and/or ROCK pathway in the cell, and that is induced in one or more pro-survival pathway(s) such that incubation time with modulator(s) of RhoA and/or ROCK pathway in the cell can be reduced and/or does not significantly affect viability of the cell. Alternatively, or in addition, the present invention utilizes a starter cell that is not sensitive to incubation with one or more modulators of SRC pathway in the cell, and that is induced in one or more pro-survival pathway(s) such that incubation time with modulator(s) of SRC pathway in the cell can be reduced and/or does not significantly affect viability of the cell. The present invention provides the induction of any cellular pro-survival pathway known in the art, or that may become known in the future such that it may be induced to enhance viability of the cell in response to incubation with dexamethasone and/or modulation of RhoA and/or ROCK pathway and/or modulation of SRC pathway and/or to reduce the culture time with dexamethasone and/or one or more other modulators of RhoA and/or ROCK pathway and/or one or more modulators of SRC pathway.

In another example, the present invention utilizes a starter cell that is sensitive to incubation of the cell with dexamethasone and/or to presence of dexamethasone in the cell, and that is induced in one or more pro-survival pathway(s) such that incubation time of the cell with dexamethasone can be reduced and/or does not significantly affect viability of the cell. The present invention provides the induction of any cellular pro-survival pathway known in the art, or that may become known in the future such that it may be induced to enhance viability of the cell in response to incubation with dexamethasone and/or to reduce the culture time with dexamethasone. By “sensitive to incubation with dexamethasone and/or to presence of dexamethasone in the cell” is taken to mean the cell undergoes cell death and/or has not activated one or more pro-survival pathway(s). For example, cell death is the result of the induction or outcome of any cellular process that includes but is not limited to necrosis, apoptosis or programmed cell death.

In one example, survival of the cell following incubation with dexamethasone and/or to presence of dexamethasone in the cell as described in any example herein is enhanced by activating the Akt/(PKB) pathway also referred to as protein kinase B (PKB). In another example, survival of the cell following incubation with dexamethasone and/or to presence of dexamethasone in the cell as described in any example herein is enhanced by activating the NF-κB pathway.

In another example, the present invention utilizes a starter cell that is sensitive to incubation with one or more modulators of RhoA and/or ROCK pathway and/or to modulation of RhoA and/or ROCK pathway in the cell, and that is induced in one or more pro-survival pathway(s) such that incubation time with modulator(s) of RhoA and/or ROCK pathway or modulation time of RhoA and/or ROCK pathway in the cell can be reduced and/or does not significantly affect viability of the cell. The present invention provides the induction of any cellular pro-survival pathway known in the art, or that may become known in the future such that it may be induced to enhance viability of the cell in response to modulation of RhoA and/or ROCK protein(s) activity and/or to reduce the culture time with a modulator of RhoA and/or ROCK pathway. By “sensitive to incubation with one or more modulators of RhoA and/or ROCK pathway and/or to modulation of RhoA and/or ROCK pathway in the cell” is taken to mean undergoes cell death and/or has not activated one or more pro-survival pathway(s). For example, cell death is the result of the induction or outcome of any cellular process that includes but is not limited to necrosis, apoptosis or programmed cell death.

In another example, the present invention utilizes a starter cell that is sensitive to incubation with one or more modulators of SRC pathway and/or to modulation of SRC pathway in the cell, and that is induced in one or more pro-survival pathway(s) such that incubation time with modulator(s) of SRC pathway or modulation time of SRC pathway in the cell can be reduced and/or does not significantly affect viability of the cell. The present invention provides the induction of any cellular pro-survival pathway known in the art, or that may become known in the future such that it may be induced to enhance viability of the cell in response to modulation of SRC activity and/or to reduce the culture time with a modulator of SRC pathway. By “sensitive to incubation with one or more modulators of SRC pathway and/or to modulation of SRC pathway in the cell” is taken to mean undergoes cell death and/or has not activated one or more pro-survival pathway(s). For example, cell death is the result of the induction or outcome of any cellular process that includes but is not limited to necrosis, apoptosis or programmed cell death.

In one example, survival of the cell following incubation with one or more modulators of SRC pathway and/or to modulation of SRC pathway in the cell as described in any example herein is enhanced by activating the Akt/(PKB) pathway. In another example, survival of the cell following incubation with a modulator of SRC and/or to modulation of SRC in the cell as described in any example herein is enhanced by activating the NF-κB pathway.

In one example, survival of the cell following incubation with one or more modulators of RhoA and/or ROCK pathway and/or to modulation of RhoA and/or ROCK pathway in the cell as described in any example herein is enhanced by activating the Akt/(PKB) pathway. In another example, survival of the cell following incubation with a modulator of RhoA and/or ROCK pathway and/or to modulation of RhoA and/or ROCK pathway in the cell as described in any example herein is enhanced by activating the NF-κB pathway.

In one example, the present invention utilizes a starter cell that is not sensitive to low-serum culture conditions used by the method of the invention. For example, such a starter cell is not sensitive to culturing in low serum for the period the cell is required to be maintained in low serum conditions. By “not sensitive to low-serum culture conditions” is taken to mean does not undergo cell death and/or has activated one or more pro-survival pathway(s). For example, cell death is the result of the induction or outcome of any cellular process that includes but is not limited to necrosis, apoptosis or programmed cell death. In another example, the present invention utilizes a starter cell that is not sensitive to low-serum culture conditions, and that is induced in one or more pro-survival pathway(s) such that the incubation time in low-serum can be reduced. The present invention provides the induction of any cellular pro-survival pathway known in the art, or that may become known in the future such that it may be induced to reduce the culture time in low serum.

In another example, the present invention utilizes a starter cell that is sensitive to low-serum culture conditions, and induced in one or more pro-survival pathway(s) such that the incubation time in low-serum can be reduced. The present invention provides the induction of any cellular pro-survival pathway known in the art, or that may become known in the future such that it may be induced to reduce the culture time in low serum. By “sensitive to low-serum culture conditions” is taken to mean undergoes cell death and/or has not activated one or more pro-survival pathway(s). For example, cell death is the result of the induction or outcome of any cellular process that includes but is not limited to necrosis, apoptosis or programmed cell death. In one example, the culture time in low-serum as described in any example herein is reduced by any time less than 7 days by activating the Akt/(PKB) pathway. In another example, the culture time in low-serum as described in any example herein is reduced by activating the NF-κB pathway.

In one example, the present invention utilizes a starter cell that is not sensitive to high cell density culture conditions used by the method of the invention. For example, such a starter cell is not sensitive to incubation at incubation of cells at a starting density of detached cells of about 1500 cells/mm² plating surface area to about 200,000 cells/mm² plating surface area or greater, including about 1,850 cells/mm² surface area of the culture vessel or greater, or about 2,220 cells/mm² surface area of the culture vessel or greater, or about 2,590 cells/mm² surface area of the culture vessel or greater, or about 2,960 cells/mm² surface area of the culture vessel or greater, or about 2,220 cells/mm² surface area of the culture vessel or greater, or about 3,330 cells/mm² surface area of the culture vessel or greater, or about 3,703 cells/mm² surface area of the culture vessel surface area of the culture vessel or greater, or about 7,407 cells/mm² surface area of the culture vessel surface area of the culture vessel or greater. By “not sensitive to high cell density culture conditions” is taken to mean does not undergo cell death and/or has activated one or more pro-survival pathway(s). For example, cell death is the result of the induction or outcome of any cellular process that includes but is not limited to necrosis, apoptosis or programmed cell death. In another example, the present invention utilizes a starter cell that is not sensitive to high cell density culture conditions, and that is induced in one or more pro-survival pathway(s) such that the incubation at high cell density condition does not affect survival of the cell. The present invention provides the induction of any cellular pro-survival pathway known in the art, or that may become known in the future such that it may be induced to survival of the cell under high cell density conditions.

In another example, the present invention utilizes a starter cell that is sensitive to high cell density culture conditions, and induced in one or more pro-survival pathway(s) such that the incubation time in high cell density conditions can be reduced and/or the survival of the cell in high density conditions is enhanced. The present invention provides the induction of any cellular pro-survival pathway known in the art, or that may become known in the future such that it may be induced to reduce the culture time in high cell density culture conditions, and/or enhance survival of the cell under such conditions. By “sensitive to high density culture conditions” is taken to mean undergoes cell death and/or has not activated one or more pro-survival pathway(s). For example, cell death is the result of the induction or outcome of any cellular process that includes but is not limited to necrosis, apoptosis or programmed cell death. In one example, survival of the cells in high density culture conditions as described in any example herein is enhanced by activating the Akt/(PKB) pathway. In another example, survival of the cells in low-serum in high density culture conditions as described in any example herein is enhanced by activating the NF-κB pathway.

### 1.2. Modulation of SRC Signalling Pathway

Without being bound by any theory or mode of action, the inventor has speculated that SRC tyrosine kinases or SRC phosphorylate their target proteins at their tyrosine residues, and the resulting phosphorylation, in turn, increases or decreases the rate of the signalling pathway in which the protein target plays a regulatory role.

The method of modulating SRC pathway in a starter cell may comprise contacting the starter cell with any one or more factors that modulate(s) the phosphorylation of the Src family member protein e.g., the protein tyrosine kinase c-Src or Fyn protein or Yes protein thereby increasing or decreasing the rate at which an Src family member protein phosphorylates any one of its numerous protein targets. The term “modulation of SCR pathway” is not limited by the mechanism underlying how the rate at which phosphorylation of the Src family member protein is increased or decreased. Alternatively, or in addition the term “modulation of SCR pathway” is not limited by the mechanism underlying how the rate at which the Src family member protein phosphorylates any one of its numerous protein targets is increased or decreased. The potential mechanisms through which such a modulatory compound may act include, but are not limited to, allosteric mechanisms that affect, directly or indirectly SRC activity, as well as mechanisms that act, directly or indirectly, to promote the phosphorylation of the SRC catalytic subunit catalyzed by upstream kinase e.g., a protein tyrosine phosphatase (PTB) such as protein tyrosine phosphatase 1B (PTB1B), or by collagen I .

In one example, the modulator is an inducer that initiates and/or enhances activation of the SRC in a starter cell. Such an inducer is also referred to as SRC enhancer or an SRC agonist. For example, the inducer is a peptide, a polypeptide, a chemical, a nucleic acid, an antibody, an antibody fragment or a small molecule, or any insult that induces cellular stress such as, but not limited to by nitric oxide hypoxia conditions.

The present invention provides any inducer of SRC signalling known in the art or to be developed in the future. Preferably, the inducer of SRC signalling includes, but is not limited to factors such as transforming growth factor (TGF) and/or transforming growth factor beta-1 (TGF-β1) and/or nerve growth factor e.g., nerve growth factor beta (NGFβ) and/or interleukin 1-β (IL-1β) and/or Fibroblast growth factor-1 (FGF-1) and/or fibroblast growth factor-2 (FGF-2) and/or hepatocyte growth factor (HGF) and/or neurotrophin 3 (NT3) and/or a semaphorin (SEMA) such as SEMA-3A and/or a platelet-derived growth factor (PDGF) such as platelet-derived growth factor B-chain (PDGF-B) or any active fragment or any active chemical group thereof.

A method for inducing SRC signalling with TGF-β1 includes as described in Seok Soon Park et al., Oncogene, 23: 6272-6281 (2004) or any references as described therein. A method for inducing SRC signalling with NGF includes as described in Wooten et al., Mol. Cell. Biol. 21:8414-8427 (2001) or in Vambutas et al., J. Biol. Chem. 270(43): 25629-25633 (1995) or any references as described therein. A method for inducing SRC signalling with IL-1β includes as described in Viviani et al., J. Neurosci. 23(25): 8692-8700 (2003) or in Davis et al., Journal of Neurochemistry. 98(5):1379-1389 (2006) or any references as described therein. A method for inducing SRC signalling with FGF-1 includes as described in Zhan et al., J Bio Chem. 269(32):20221-20224 (1994) or any references as described therein. A method for inducing SRC signalling with FGF-2 includes as described in Kanda et al., Exp. Cell Res. 312:3015-3022 (2006) or in Shono et al., Exp. Cell Res. 264: 275-283 (2001) or Li et al., J. Cell Sci. 117(25):6007-6017 or any references as described therein. A method for inducing SRC signalling with HGF includes as described in Maejima et al., Atherosclerosis. 167 (1): 89-95 or any references as described therein. A method for inducing SRC signalling with NT3 includes as described in Yamauchi et al., PNAS 100(24): 14421-14426 (2003) or in Yamauchi et al., PNAS 102(41): 14889-14894 (2005) or any references as described therein. A method for inducing SRC signalling with SEMA-3A includes as described in Acevedo et al., Blood. 111(5): 2674-2680 (2008) or any references as described therein. A method for inducing SRC with PDGF-B includes as described in Takikita-Suzuki et al., Am J Pathol. 163(1): 277-286 (2003) or any references as described therein.

In another preferred example, the inducer of SRC signaling includes activating any intracellular signalling intermediate that activates and/or enhances the SRC signaling pathway by contacting a starter cell with a peptide, a polypeptide, a chemical, a nucleic acid, an antibody, an antibody fragment and/or a small molecule, such that the activation initiates and/or enhances the SRC signaling pathway. These intracellular intermediates include but are not limited to downstream signalling intermediates activated by growth factor receptors such as EGFR and/or PDGF-R and/or FGFR3 and/or ras and/or downstream signalling intermediates activated by GPCR receptors such as TrKA and/or P2Y2, and/or downstream signalling intermediate activated by steroid receptors such as SRC-1.

In another example, the inducer of SRC pathway includes activating any receptor that initiates and/or enhances SRC signalling by contacting the receptor of a starter cell with a peptide, a polypeptide, a chemical, a nucleic acid, an antibody, an antibody fragment and/or a small molecule, such that the receptor activation modulates the SRC activation and/or function. In one example, the receptor includes but not limited to EGFR and/or VEGF and/or PDGFR and/or or the c-Met receptor also known as PTKR and/or FGFR.

In another example, the modulator is an inhibitor that inhibits and/or suppresses activation and/or function of the SRC in a starter cell. Such an inhibitor is also referred to as SRC suppressor or an SRC antagonist. For example, the inhibitor is a peptide, a polypeptide, a chemical, a nucleic acid, an antibody, an antibody fragment or a small molecule, and/or any condition or insult that induces cellular stress such as hypoxia.

The present invention provides any inhibitor of the SRC signalling pathway known in the art or to be developed in the future. Preferably, the inhibitor of SRC signalling includes, but is not limited to factors such as 4-amino-5-(4-methylphenyl)-7-(t-butyl)pyrazolo (3,4-d) pyrimidine, (PP1) and/or 4-Amino-5-(4-chlorophenyl)-7-(t-butyl)pyrazolo[3,4-d]pyrimidine (PP2) and/or TGF-β1 and/or SU6656 and/or UCS15A. A method for inhibiting SRC signalling with PP1 includes as described in Mukherjee et al., Mol. Cancer, 7: 37 (2008) or any references as described therein. A method for inhibiting SRC signalling with PP2 includes as described in Peng and Schoenberg, Mol. Cell. 25(5): 779-787 (2007) or any references as described therein. A method for inhibiting SRC signalling with TGF-β1 includes as described in Leontiou et al., Endocrine Abstracts 13:P205 (2007) or any references as described therein. A method for inhibiting SRC signalling with SU6656 includes as described in Li et al., Journal of Cell Science 117(25):6007-6017 (2004) or any references as described therein. A method for inhibiting SRC signalling with UCS15A includes as described in Sharma et al., Oncogene 20(17): 2068-2079 (2001) or any references as described therein.

In another preferred example, the inhibitor of SRC signaling includes inhibiting and/or suppressing any intracellular signalling intermediate that activates and/or enhances the SRC signaling pathway by contacting a starter cell with a peptide, a polypeptide, a chemical, a nucleic acid, an antibody, an antibody fragment and/or a small molecule, such that the intracellular signalling intermediate activation and/or enhancement of SRC is inhibited and/or suppressed. These intracellular intermediates include but are not limited to downstream signalling intermediates activated by growth factor receptors such as, EGFR and/or PDGF-R and/or FGFR3 and/or ras and/or downstream signalling intermediates activated by GPCR receptors such as TrKA and/or P2Y2, and/or downstream signalling intermediate activated by steroid receptors such as SRC-1.

In another example, the inhibitor of SRC pathway includes inhibiting and/or suppressing any receptor that initiates and/or enhances SRC pathway by contacting the receptor of a starter cell with a peptide, a polypeptide, a chemical, a nucleic acid, an antibody, an antibody fragment and/or a small molecule, such that the receptor activation and/or enhancement of SRC pathway is inhibited and/or suppressed. Preferably, the receptors include but not limited to EGFR and/or VEGF and/or PDGFR and/or or the c-Met receptor also known as PTKR and/or or FGFR.

In one example, inducing or enhancing SRC pathway in the starter cell is under conditions sufficient to induce or enhance the SRC pathway but not sufficient to induce or enhance the Akt/(PKB) pathway and/or the NF-κB pathway. In another example, inhibition or suppression of SRC in the starter cell is under conditions sufficient to inhibit or suppress the SRC but not sufficient to inhibit or suppress the Akt/(PKB) pathway and/or the NF-κB pathway. To achieve such specificity or selectivity, it is preferred to employ one or more modulators of the SRC pathway that do not initiate or engage in cross-talk with the Akt/(PKB) pathway and/or the NF-κB pathway. For example, one or more modulators of the SRC pathway is employed in performing the present invention that do not also modulate i.e, activate or inhibit or suppress the Akt/(PKB) pathway. In accordance with this example, it is particularly preferred that a modulator of the SRC pathway employed in performing the present invention according to any example hereof is other than a TGF e.g., TGF-β, and/or is other than an IL-1 e.g., IL-1β and/or is other than PDGF e.g., PDGF-B or PDGF-BB and/or is other than a NGF e.g., NGF-κB. In another example, one or more modulators of the SRC pathway is employed in performing the present invention that do not also modulate i.e, activate or inhibit or suppress the NF-κB pathway. In accordance with this example, it is particularly preferred that a modulator of the SRC pathway employed in performing the present invention according to any example hereof is other than an IL-1 e.g., IL-1β.

In another example, inducing or enhancing SRC pathway in the starter cell is for a time and under conditions sufficient to induce or enhance the SRC pathway but not also sufficient to induce or enhance the 5′ AMP-activated protein kinase or AMPK pathway. In another example, inhibition or suppression of SRC in the starter cell is for a time and under conditions sufficient to inhibit or suppress the SRC but not also sufficient to inhibit or suppress the AMPK pathway.

A method to measure the modulation of SRC pathway includes any method that measures the activity of SRC, or any known intracellular signaling intermediate of the SRC, as described in Wooten et al., Mol. Cell. Biol. 21:8414-8427 (2001), or any reference described therein. For example, SRC activity may be determined using in vitro kinase assays by incubating SRC e.g., from cell lysates with peptide substrate (e.g., KVRKRGEGTYGVVKK) or protein substrate (e.g., acid-denatured enolase) in the presence of γ-³²P ATP plus buffers, and divalent cations (Mn²+ and Mg²+). The kinase reaction is terminated by boiling the reaction mixtures in Laemmli sample buffer. The reaction mixture is then electrophoresed to separate the substrate from the unincorporated ³²P. Finally, Src activity is determined by measuring the quantity by autoradiography or scintillation counting. For example, SRC activity may be determined by assays with acid-treated enolase as the substrate as described in Cooper et al., J. Biol. Chem. 271:5325-5331 (1996) or any references as described therein or Kanda et al., J. Biol. Chem. 275: 10105-10111 (2000) or any references as described therein, which are incorporated herein by reference. Briefly, about 2 μg of mouse anti-SRC antibody is added to cell lysate and incubated with anti-mouse IgG agarose to allow immune complex containing SRC to form. The complexes are washed with immunoprecipitation kinase wash buffer (e.g., 35 mM Tris-HCL [pH 7.4], 150 mM NaCl, 15 mM MgCl₂, 1 mM MnCl₂, 0.5 mM EGTA, 0.1% TX-100, 25 μg of leupeptin/ml, 25 μg of aprotinin/ml) and then with SRC immunoprecipitation kinase assay buffer (20 mM HEPES [pH 7.0], 10 mM MnCl, 0.05% TX-100). The immune complexes are then suspended in SRC immunoprecipitation kinase assay buffer containing about 1 μg of acid-treated enolase. The kinase reaction is initiated by addition of about [γ-³²P]ATP, and the mixture is incubated at 30° C. for about 10 min. The reaction is stopped by addition of 2×SDS-PAGE sample buffer [8% SDS, 0.4 M Tris-HCl (pH 8.8), 1 M sucrose, 10 mM EDTA, 0.02% bromophenol blue, and 4% 2-mercaptoethanol]. The radiolabelled proteins are resolved by SDS-PAGE gels, and exposed to X-ray films (e.g., Fuji).

Alternatively or in addition thereto, Src family member protein or SRC activity can be detected by monitoring the phosphorylation state of one of its substrate proteins including but not limited to FAK and/or Vinculin and/or Cortactin and/or Paxillin and/or Tensin and/or Ezrin and/or p130cas and/or p190RhoGAP and/or p120RasGap according to any methods known in the art, or by measuring the phosphorylation and activation of the transcription factors STAT1 and/or STAR3 and/or STAT5. SRC activity studies using STAT1 and/or STAR3 and/or and STAT5 are described in Qing et al., J. Biol. Chem. 279:41679-41685 (2004) and in Xi et al., J. Biol. Chem. 278:31574-31583 (2001) or any reference described therein, incorporated herein by reference. Alternatively or in addition, SRC activity can be followed using the commercially available PhosphoELISA™ kit (Invitrogen; catalogue Nos: KHB3441 and KHB3482) according to manufacturer's instructions, which may be employed to monitor activity of SRC protein using activation of the transcription factors STAT1 and/or [pY701], e.g., by employing affinity purified rabbit polyclonal antibody specific to STAT1 phosphorylated tyrosine 701 residue.

In one example, the present invention provides that the starter cells are incubated in the presence of a modulator of SRC pathway for a time and under condition for time sufficient to render the cells capable of being differentiated into a plurality of different cell types. It will be apparent to the skilled artisan that the time of incubation in the presence of a modulator SRC pathway may vary according to cell type and/or according to the type of modulator, and it is well within the ken of a skilled addressee to determine such parameters without undue experimentation. For example, the time of incubation in the presence of the modulator of SRC pathway is between about 5 min and about 48 hours. Preferably, the time of incubation in the presence of the modulator is between about 5 min and about 24 hours, or preferably between about 5 min and about 15 hours, or preferably between about 5 min and 10 hours, or preferably between about 5 min and about 8 hours, or preferably between about 5 min and about 6 hours, or preferably between about 5 min and about 4 hours, or preferably between about 5 min and about 3 hours, or preferably between about 5 min and about 2 hours, or preferably between about 5 min and about 1 hour, or preferably between about 10 min and about 1 hour, or preferably between about 15 min and about 30 min. The person skilled in the art would appreciate that production of progenitor cells may continue albeit at below optimum even after the 48 hours incubation in the presence of a modulator of SRC pathway, however such sub-optimum incubation conditions are clearly within the scope of the invention. In one example the amount of modulator used for incubation according to any embodiment hereof is between 0.001 μM and 100 μM. Preferably the amount of modulator is between 0.01 μM and 10 μM, or preferably between 0.1 μM and 10 μM.

1.3. Modulation of RhoA and/or ROCK Signalling Pathway

Without being bound by any theory or mode of action, the inventor has reasoned that the small GTPase RhoA mediates its effect on the differentiated cells by its downstream effector Rho-associated kinase (ROCK) in response to GTP levels in the cell, such that GTP binding and hydrolysis switches RhoA between a GTP-bound conformationally active and GDP-bound inactive state. The conformationally active RhoA in-turn propagates downstream signals by binding to effector proteins such as ROCK that phosphorylates its target proteins, and the resulting phosphorylation in turn may increase or decrease the rate of the signalling pathway in which the protein target plays a regulatory role. “ROCK” is also known in the art as p160ROCK or Rho kinase.

The method of modulating RhoA and/or ROCK in a starter cell may comprise contacting the starter cell with any one or more factors that modulate(s) the RhoA switching between its GTP-bound conformationally active and GDP-bound inactive state and/or ROCK phosphorylation, thereby increasing or decreasing the rate at which ROCK phosphorylates any one of its numerous protein targets. Modulating of RhoA and/or ROCK is not limited by the mechanism underlying how the rate at which RhoA and/or ROCK activates and/or phosphorylates any one of its protein targets is increased or decreased. The potential mechanisms through which such a compound may act include, but are not limited to, allosteric mechanisms that affect, directly or indirectly RhoA and/or ROCK activity, as well as mechanisms that act, directly or indirectly, to promote the phosphorylation of the ROCK catalytic subunit catalyzed by an upstream kinase e.g., the myosin binding subunit of myosin phosphatase (MYPT1).

In one example, the modulator is an inducer that initiates and/or enhances activation of the RhoA and/or ROCK in a starter cell. Such an inducer is also referred to as RhoA and/or ROCK enhancer or RhoA and/or ROCK agonist. For example, the inducer is a peptide, a polypeptide, a chemical, a nucleic acid, an antibody, an antibody fragment or a small molecule, and/or any insult that induces cellular stress such as but not limited to by cytoskeleton tension resulting from adhesion e.g., integrin-mediated adhesion to the extracellular matrix (ECM).

The present invention provides an inducer of RhoA and/or ROCK signalling pathway known in the art or to be developed in the future. Preferably, the inducer of RhoA and/or ROCK signalling includes, but is not limited to dexamethasone (DEX) and/or growth hormone (GH) and/or tumor Necrosis factor-α (TNF) and/or fibronectin and/or lysophosphatidic acid and/or serum or any active fragment or any active chemical group thereof.

A method for incubating cells with dexamethasone or alternatively inducing RhoA and/or ROCK pathway with dexamethasone includes as described in Chen et al., J. Biol. Chem., 278(5): 2807-2818 (2003) or any references as described therein. A method for inducing RhoA and/or ROCK pathway with GH includes as described in Ling and Lodie, J. Biol. Chem. 279(31): 32737-32750 (2004) or any references as described therein. A method for inducing RhoA and/or ROCK pathway with serum includes as described in Alblas et al., Mol. Biol. Cell. 12:2137-2145 (2001) or any references as described therein. A method for inducing RhoA and/or ROCK pathway with fibronectin or with lysophosphatidic acid includes as described in Ren et al., EMBO J., 18:578-585 (1999) or any references as described therein, which are all incorporated herein by reference.

In another preferred example, the inducer of RhoA and/or ROCK signaling pathway includes activating any intracellular signalling intermediate that activates and/or enhances the RhoA and/or ROCK signaling pathway by contacting a starter cell with a peptide, a polypeptide, a chemical, a nucleic acid, an antibody, an antibody fragment or a small molecule, such that the activation initiates and/or enhances the RhoA and/or ROCK signaling pathway. These intracellular intermediates include but are not limited to PKC, or downstream signalling intermediates activated by growth hormone receptors such as Estrogen receptor-α.

In another example, the inducer of RhoA and/or ROCK pathway includes activating any receptor that initiates and/or enhances RhoA and/or ROCK pathway by contacting the receptor of a starter cell with a peptide, a polypeptide, a chemical, a nucleic acid, an antibody, an antibody fragment or a small molecule, such that the receptor activation modulates the SRC activation and/or function. In one example, the receptors include but not limited to GPCR.

In another example, the modulator is an inhibitor that inhibits and/or suppresses activation and/or function of RhoA and/or ROCK in a starter cell. Such an inhibitor is also referred to as RhoA and/or ROCK suppressor or RhoA and/or ROCK antagonist. For example, the inhibitor is a peptide, a polypeptide, a chemical, a nucleic acid, an antibody, an antibody fragment or a small molecule, or any condition or insult that induces cellular stress such as but not limited to by cytoskeleton tension.

The present invention provides any inhibitor of the RhoA and/or ROCK pathway known in the art or to be developed in the future. Preferably, the inhibitor of RhoA and/or ROCK pathway includes, but is not limited to factors such as Y-27632 (e.g., Alexis Biochemicals). A method for inhibiting RhoA and/or ROCK with Y-27632 includes as described in Zhang at al., Neuropathology & Applied Neurobiology., 34(2):231-240 (2008) or any references as described therein, which are incorporated herein by reference.

In another preferred example, the inhibitor of RhoA and/or ROCK signaling pathway includes inhibiting and/or suppressing any intracellular signalling intermediate that activates and/or enhances the RhoA and/or ROCK signaling pathway by contacting a starter cell with a peptide, a polypeptide, a chemical, a nucleic acid, an antibody, an antibody fragment and/or a small molecule, such that the intracellular signalling intermediate activation and/or enhancement of RhoA and/or ROCK pathway is inhibited and/or suppressed. These intracellular intermediates include but are not limited to PKC, or downstream signalling intermediates activated by growth hormone receptors such as Estrogen receptor-α.

In another example, the inhibitor of RhoA and/or ROCK pathway includes inhibiting and/or suppressing any receptor that initiates and/or enhances RhoA and/or ROCK pathway by contacting the receptor of a starter cell with a peptide, a polypeptide, a chemical, a nucleic acid, an antibody, an antibody fragment or a small molecule, such that the receptor activation modulates the RhoA and/or ROCK pathway is inhibited and/or suppressed. In one example, the receptors include but not limited to GPCR.

In one example, inducing or enhancing RhoA and/or ROCK pathway in the starter cell is for a time and under conditions sufficient to induce or enhance the RhoA and/or ROCK pathway but not also sufficient to induce or enhance the Akt/(PKB) pathway and/or the NF-κB pathway. In another example, inhibition or suppression of RhoA and/or ROCK in the starter cell is for a time and under conditions sufficient to inhibit or suppress the RhoA and/or ROCK but not also sufficient to inhibit or suppress the Akt/(PKB) pathway and/or the NF-κB pathway. To achieve such specificity or selectivity, it is preferred to employ one or more modulators of the RhoA and/or ROCK pathway that do not initiate or engage in cross-talk with the Akt/(PKB) pathway and/or the NF-κB pathway. For example, one or more modulators of the RhoA and/or ROCK pathway is employed in performing the present invention that do not also modulate i.e, activate or inhibit or suppress the NF-κB pathway. In accordance with this example, it is particularly preferred that a modulator of the RhoA and/or ROCK pathway employed in performing the present invention according to any example hereof is other than a TNF-α and/or is other than lysophosphatidic acid (LPA).

A method to measure the modulation of RhoA and/or ROCK pathway includes any method that measures the activity of RhoA and/or ROCK, or any known intracellular signaling intermediate of the RhoA and/or ROCK pathway, as described in Alblas et al., Mol. Biol. Cell. 12:2137-2145 (2001) or in Hunter et al., Mol Pharmacol 63:714-721 (2003) or Ling and Lobie J. Biol. Chem., 279 (31):32737-32750 (2004) or any reference described therein. For example, RhoA and/or ROCK activity may be determined by RhoA activation assay by immunoprecipitation of RhoA-GTP proteins produced in the test cells wherein following incubation of the cells with a modulator of RhoA and/or ROCK according to any example described herein, the cells are then lysed e.g., in lysis buffer (e.g., 50 mM Tris pH 7.5, 150 mM NaCl, 10% glycerol, 1% NP-40, 0.1% Triton X-100, 5 mM MgCl₂, 0.1 mM phenylmethylsulfonyl fluoride, 10 mg/ml leupeptine, 10 mg/ml aprotinin). Cleared lysates are affinity-precipitated by incubation with bacterially produced GST-rhotekin-RBD fusion proteins (Reid et al., J. Biol. Chem. 271:13556-13560 (1996)) bound to glutathion-agarose beads for 45 min at 4° C. The beads/precipitates are washed three times with lysis buffer then bound RhoA-GTP proteins can be eluted in SDS-sample buffer/Laemmli sample buffer and analyzed by Western blotting with the use of anti-RhoA monoclonal antibody e.g., from Santa Cruz Biotechnology. RhoA activity may be determined as the amount of rhotekin-bound RhoA (GTP-RhoA) compared with the total amount of RhoA in cell lysates. Increases in RhoA activation for each experiment can be expressed as fold increases from control (zero time point), normalized to “1”.

In one example, the present invention provides that the starter cells are incubated in the presence of dexamethasone and/or one or more other modulators of RhoA and/or ROCK pathway for a time and under condition sufficient to render the cells capable of being differentiated into a plurality of different cell types. It will be apparent to the skilled artisan that the time of incubation in the presence of dexamethasone and/or one or more other modulators of RhoA and/or ROCK pathway may vary according to cell type and/or according to the type of modulator, and it is well within the ken of a skilled addressee to determine such parameters without undue experimentation. For example, the time of incubation in the presence of the dexamethasone and/or one or more other modulators of RhoA and/or ROCK pathway is between about 5 min and about 48 hours. Preferably, the time of incubation in the presence of dexamethasone and/or one or more other modulators of RhoA and/or ROCK pathway is between about 5 min and about 24 hours, or preferably between about 5 min and about 15 hours, or preferably between about 5 min and 10 hours, or preferably between about 5 min and about 8 hours, or preferably between about 5 min and about 6 hours, or preferably between about 5 min and about 4 hours, or preferably between about 5 min and about 3 hours, or preferably between about 5 min and about 2 hours, or preferably between about 5 min and about 1 hour, or preferably between about 10 min and about hour, or preferably between about 15 min and about 30 min. The person skilled in the art would appreciate that production of progenitor cells may continue albeit at below optimum even after the hours incubation in the presence of dexamethasone and/or one or more other modulators of RhoA and/or ROCK pathway, however such sub-optimum incubation conditions are clearly within the scope of the invention. In one example the amount of dexamethasone and/or one or more other modulators of RhoA and/or ROCK pathway used for incubation according to any embodiment hereof is between 0.001 μM and 100 μM. Preferably the amount of dexamethasone or modulator is between 0.01 μM and 10 μM, or preferably between 0.1 μM and 10 μM.

### 1.4. Induction of the Akt/(PKB) Pathway

In one example, the method to induce the Akt/(PKB) pathway in a starter cell may comprise contacting the starter cell with any one or more factors that induce(s) the Akt/(PKB) signaling pathway. For example, an inducer initiates and/or enhances Akt/(PKB) pathway signaling in a starter cell. Such an inducer is also referred to as an Akt/(PKB) pathway enhancer or an Akt/(PKB) pathway agonist. For example, the inducer is a peptide, a polypeptide, a chemical, a nucleic acid, an antibody, an antibody fragment or a small molecule, or any insult that induces cellular stress such as, but not limited to, hypoxia, or UV irradiation.

The present invention provides any inducer of the Akt/(PKB) signaling known in the art or to be developed in the future. Preferably, the inducer of Akt/(PKB) signaling includes, but is not limited to factors such as: platelet derived growth factor (PGDF-BB), insulin growth factor (IGF-1), transforming growth factor-beta (TGF-β), nerve growth factor (NGF) and carbachol, pyruvate, cytokines such as IL-1, or any active fragment or active chemical group thereof. A method for inducing the Akt/(PKB) pathway with PDGF-BB includes as described in Li et al., Mol. Biol. Cell 15:294-309 (2004) or Gao et al, J. Biol. Chem. 280:9375-9389 (2005) or any references as described therein. A method for inducing the Akt/(PKB) pathway by co-activation with carbachol and NGF includes as described in Wu and Wong Cellular Signalling 18:285-293 (2006) or any references as described therein. A method for inducing the Akt/(PKB) pathway with IGF-1 includes as described in Kulik and Weber Mol. Cell. Biol. 18:6711-6718 (1998) or any reference as described therein. A method for the induction of the Akt/(PKB) pathway by TGF-β includes as described in by Conery et al., Nat Cell Biol (2004) 6: 366-72 or as described by Horowitz et al., J. Biol. Chem. 279: 1359-1367 (2004) or any reference as described therein. Other methods for inducing the Akt/(PKB) pathway with these factors includes methods as described in any one of the Examples or as described in Song et al., J. Cell. Mol. Med. 9:59-7 (2005); Dillon et al., Oncogene 26:1338-1345 (2007) or any reference as described therein.

In another preferred example, the inducer of Akt/(PKB) signaling includes activating a receptor that initiates and/or enhances the Akt/(PKB) signaling pathway by contacting the receptor of a starter cell with a peptide, a polypeptide, a chemical, a nucleic acid, an antibody, an antibody fragment or a small molecule, such that the receptor activation initiates and/or enhances the Akt/(PKB) signaling pathway.

Preferably, the receptor is a growth factor receptor such as IGF receptor tyrosine kinase, or the TGF-β type I serine/threonine kinase receptor, or the TGF-β type II serine/threonine kinase receptor, or TGF-β type III receptor, or any one of the integrin receptors, such as α2β1, α1β1, or αv3, or a GPCR receptor, or a cytokine receptor such as the IL-1 receptor, or a B-cell receptor.

In another preferred example, the inducer of Akt/(PKB) signaling includes activating any intracellular signalling intermediate that initiates and/or enhances the Akt/(PKB) signaling pathway by contacting a starter cell with a peptide, a polypeptide, a chemical, a nucleic acid, an antibody, an antibody fragment or a small molecule, such that the activation initiates and/or enhances the Akt/(PKB) signaling pathway. These intracellular intermediates include but are not limited to downstream signalling intermediates activated by growth factor receptors such as GAB1 and/or GAB2 and/or IRS1 and/or PI3K, and/or PIP2 and/or PIP3 and/or ras and/or or downstream signalling intermediates activated by integrin receptors such as FAK and/or paxillin and/or ILK and/or PI3K and/or PIP2 and/or PIP3 and/or or downstream signalling intermediates activated by cytokine receptors such as JAK1 and/or PI3K and/or PIP3 and/or PDK-1 or downstream signalling intermediates activated by B-cell receptors such as BCAP and/or PI3K and/or PDK-1 and/or downstream signalling intermediates activated by GPCR receptors such as GβGγ/PI3K and/or PIP3 and/or PDK-1.

A method to measure the activation of the Akt/(PKB) pathway includes any method that measures the activity of Akt/(PKB), or any known intracellular signaling intermediate of the Akt/(PKB), as described in Kulik and Weber Mol. Cell. Biol. 18:6711-6718 (1998), or any reference described therein. For example, the phosphorylation of Akt/(PKB) may be used as a marker of the activation of the pathway. The method to measure Akt/(PKB) phosphorylation is described in Kulik and Weber. Briefly, after incubation with factors to induce the Akt/(PKB) pathway, cells are placed on ice and lysed in 1% Nonidet P-40, 0.5% deoxycholate, 150 mM NaCl, and 20 mM HEPES supplemented with phosphatase and protease inhibitors. Insoluble material is pelleted by centrifugation at 10,000×g for 20 min, and the supernatants are equalized for protein concentration by the addition of NLB. Samples are subjected to Western Blot analysis by standard methods using a phospho-Akt (S473) specific antibody. The membrane is stripped and reprobed with Akt-specific antibodies.

### 1.5. Induction of the NF-κB Pathway

In one example, the method to induce the NF-κB pathway in a starter cell may comprise contacting the starter cell with any one or more factors that induce(s) the NF-κB signaling pathway in said primary cell, cell strain or cell line. For example, an inducer initiates and/or enhances NF-κB pathway signaling in a starter cell. Such an inducer is also referred to as an NF-KB pathway enhancer or an NF-κB pathway agonist.

The present invention provides any inducer of NF-κB signaling known in the art or to be developed in the future. For example, the inducer is a peptide, a polypeptide, a chemical, a nucleic acid, an antibody, an antibody fragment or a small molecule, or any insult that induces cellular stress such as, but not limited to, hypoxia, UV irradiation, or high cell density culturing, maintenance or incubation.

Preferably, the inducer of NF-κB signaling includes, but is not limited to factors such as: tumor necrosis factor-alpha (TNF-α), interleukin 1 (IL-1), or any active fragment thereof, lysophosphatidic acid (LPA), pyruvate, or lipopolysaccharide (LPS). A method for inducing the NF-κB signaling pathway with TNF-a includes as described in Kouba et al., J. Biol. Chem. 276:6214-6244 (2001) or any reference as described therein. A method for inducing the NF-κB signaling pathway with IL-1 includes as described in Kessler et al., J. Exp. Med. 176:787-792 (1992) or any reference as described therein. A method for inducing the NF-κB signaling pathway with LPA includes as described in Shahrestanifar et al., J. Biol. Chem. 274:3828-3833 (1999) or any reference as described therein. Other methods for inducing the NF-κB signaling pathway with any of these factors includes as described in any one of the examples.

In another preferred example, the inducer of NF-κB signaling includes activating a receptor that initiates and/or enhances the NF-κB signaling pathway by contacting the receptor of a starter cell with a peptide, a polypeptide, a chemical, a nucleic acid, an antibody, an antibody fragment and/or a small molecule, or any insult that induces cellular stress such as UV irradiation or incubating, maintaining or incubating cells at high cell density, such that the receptor activation initiates and/or enhances the NF-κB signaling pathway.

In one example, the inducer of NF-κB signaling comprises culturing, maintaining or incubating differentiated cells at high cell density conditions.

In one example, the receptor is a cytokine receptor such as the IL-1 receptor and/or the TNF receptor and/or a growth factor receptor such as the IGF receptor and/or the LPS receptor such as TLRs and/or the T-cell receptor, and/or a B-cell receptor.

In another example, the inducer of NF-κB signaling includes activating any intracellular signalling intermediate that initiates and/or enhances the NF-κB signaling pathway by contacting a starter cell with a peptide, a polypeptide, a chemical, a nucleic acid, an antibody, an antibody fragment and/or a small molecule, such that the activation initiates and/or enhances the NF-κB signaling pathway. These intracellular intermediates include but are not limited to downstream signalling intermediates activated by growth factor receptors such as PI3K and/or Akt/PKB and/or by the TNF receptor(s) such as TRADD/RIP/FADD/TRAF or NIK/MEKK and/or the cytokine receptors such as TRAF6/MyoD/IRAK, IRAK/TRAF6, TAK1 and/or T-cell receptors such as Vav/PKC/ZAP70, BIMP/BCL10/MALT and/or B-cell receptors such as BLK/Lyn/Fyn, PKC or BIMP/BCL10/MALT.

A method to measure the activation of the NF-κB pathway includes any method that measures the activity of NF-κB such as the translocation of NF-κB. Such methods are well known in the art and includes methods as described in Ding et al., J Biol Chem, 273:28897-28905 (1998) or any reference as described therein. Briefly, cells that have been induced in their NF-κB pathway are fixed with 4% formaldehyde in phosphate-buffered saline for 20 min at room temperature, permeabilized with 0.1% Triton X-100 in phosphate-buffered saline for 5 min at room temperature, and then washed twice with 0.1 M Tris-HCl buffer, pH 7.8. To block nonspecific antigenic sites, cells are incubated for 20 min with 5% non-fat dry milk in 0.1M phosphate buffer, pH 7.8, at room temperature. Cells are washed two times in 0.1M Tris wash buffer, incubated for h with rabbit anti-p65 NF-κB antibody (Santa Cruz Biotechnology, Inc., Santa Cruz, Calif.) diluted 1:2000 in 0.1M phosphate buffer, pH 7.8, with 0.1% bovine serum albumin (fraction V; Sigma). The plates are washed three times in Tris wash buffer and incubated 30 min, room temperature, with a 10 μg/ml solution in water of biotinylated anti-rabbit IgG (Vector Laboratories, Burlingame, Calif.). The plates are washed three times in Tris wash buffer and incubated 30 min, room temperature, with 2.54 ml solution of Texas Red avidin (Vector) in the phosphate/bovine serum albumin buffer. The cells are washed three times in Tris wash buffer and stored in 0.1 M Tris. Two hours prior to analysis a 1 μg/ml solution of Hoechst 33342 (Molecular Probes, Inc., Eugene, Oreg.) in phosphate-buffered saline is added to each well at room temperature, and the wells are scanned and analysed in the ArrayScan™ cytometer (Cellomics, Inc., Pittsburgh, Pa.).

### 2. Detachment of Adherent Cells in Culture

In accordance with the generality of the invention, the means by which adherent cells in culture are detached from each other and/or from the culture vessel may be varied.

In a preferred example, adherent cultures are detached from tissue culture plates by incubation of the adherent cells in trypsin for a time and under conditions sufficient for detachment to occur e.g., as described in the Examples.

Trypsin may be purchased from a variety of commercial sources in stock concentrations up to about 2.5% (w/v) trypsin, such as, for example, from GIBCO (Invitrogen). The final trypsin concentration used to achieve detachment when using such a solution is preferably about 0.01% (w/v) to about 0.25% (w/v) trypsin, including about 0.05% (w/v), or about 0.10% (w/v), or about 0.11% (w/v), or about 0.12% (w/v), or about 0.13% (w/v), or about 0.14% (w/v), or about 0.15% (w/v), or about 0.16% (w/v), or about 0.17% (v/v) or about 0.18% (w/v) or about 0.19% (w/v) or about 0.2% (w/v) or about 0.25% (w/v).

It will be apparent to the skilled artisan that the time of incubation in trypsin solution may vary according to cell type, and it is well within the ken of a skilled addressee to determine such parameters without undue experimentation. For example, the time of incubation in trypsin solution is sufficient for the cells to lift from the plates and/or preferably, to detach from each other as determined by the degree of cell clumping or aggregation.

It will also be apparent to the skilled artisan that the temperature for the incubation in trypsin solution is preferably between about 15° C. and about 37° C., or preferably room temperature, or more preferably 37° C. By “room temperature” is meant ambient temperature e.g., between about 18° C. and about 25° C.

Other suitable methods for achieving detachment of cells from each other and/or from the culture vessel include, but are not limited to, cold shock; treatments to release integrin receptors from the extracellular matrix, which comprises fibronectin, vitronectin, and one or more collagens; activation of degradation of matrix molecules including, but not limited to fibronectin, collagens, proteoglycans, and thrombospondin; inducing or enhancing the secretion of proteases , such as, but not limited to collagenase, stromelysin, matrix-metalloproteinases (MMPs; a class of structurally related zinc-dependent endopeptidases that collectively degrade extracellular matrix components) or plasminogen activator; and decreasing or repressing the expression of protease inhibitors, plasminogen activator inhibitor (PM-1) or tissue inhibitors of metalloproteinases (TIMPs), or any combination thereof. Such methods are described without limitation for example, by Ivaska and Heino, Cell. Mol. Life. Sci. 57:16-24 (2000), Nagase et al., Cardovasc. Res. 69(3):562-73 (2006) or a reference cited therein.

Preferred cold shock means comprise incubating the cells in ice-cold phosphate buffered saline (PBS) or other isotonic buffer for a time and under conditions sufficient for detachment to occur. Preferred conditions include cold shock for about 10 minutes or until the cells lift from the plates and/or detach from each other as determined by the degree of cell aggregation.

A further suitable method for achieving detachment of cells from each other and/or from the culture vessel includes incubating the cells in a citric saline (e.g., 0.135M potassium chloride, 0.015M sodium citrate). Preferred citric saline treatment comprises incubating the cells and citric saline in PBS at 37° C. and decanting cells for a time and under conditions sufficient for cells to lift from the plates and/or detach from each other as determined by the degree of cell aggregation.

Integrin receptors can be released from the extracellular matrix by incubating the cells with a synthetic peptide containing the Arg-Gly-Asp sequence that competes for binding to the integrin receptors such as described, for example, by Haymen et al., Journal Cell Biol, 100:1948-1954 (1985). Alternatively, or in addition, integrin receptors are released from extracellular matrix by incubating cells in a Ca²⁺-free and Mg⁺-free solution comprising EDTA (e.g., Ca²⁺-free and Mgt free PBS comprising EDTA, or other Ca²⁺-free and Mgt-free isotonic buffer) essentially as described by Sambrook, Fritsch & Maniatis, Molecular Cloning: A Laboratory Manual, Cold Spring Harbor Laboratories, New York, Second Edition (1989), whole of Vols I, II, and III; Animal Cell Culture: Practical Approach, Third Edition (John R.W. Masters, ed., 2000), ISBN 0199637970, whole of text; Methods In Enzymology (S. Colowick and N. Kaplan, eds., Academic Press, Inc.), whole of series.

Preferred means for inducing or enhancing MMP expression include induction by addition of growth factor or cytokine to the culture medium.

The present invention clearly encompasses the use of any means by which adherent cells in culture are detached from each other and/or from the culture vessel as described by Ivaska and Heino, Cell. Mol. Life. Sci. 57:16-24 (2000) and references described therein.

### 3. Ligands of Protease Activated Receptors (PARs)

In an alternative example, adherent cultures are detached from tissue culture plates by incubation of the adherent cells in the presence of one or more PAR ligands for a time and under conditions sufficient for detachment to occur e.g., as described in the Examples.

By “protease-activated receptor” or “PAR” is meant any one of a class of G-protein coupled receptors including, but not limited to, the receptors designated PAR1, PAR2, PAR3, and PAR4, and combinations thereof.

Activation of a PAR by its cognate endogenous or non-endogenous ligand leads to a cascade of cellular events such as, for example, contraction of myometrium and/or vascular and/or smooth muscle and/or activation of mitogen-activated protein kinases as described e.g., by Shintani et al., British Journal of Pharmacology (2001) 133, 1276-1285 or Belham et al., Biochem J. 320: 939-946, 1996. Alternatively, or in addition, activation of PAR by the ligand may protect cells from apoptosis and/or activate the Akt/(PKB) pathway and/or activate the NF-κB pathway. Activation of the Akt/(PKB) pathway and/or activation of the NF-κB pathway can be determined e.g., by detecting expression of one or more pathway intermediates in cells.

Preferred PAR ligands include, but are not limited to trypsin, tryptase, chymotrypsin, elastase, thrombin, plasmin, coagulation factor Xa, granzyme A and cathepsin G.

PAR ligands that are proteases can be purchased from a variety of commercial sources and used, for example at concentrations in the range of about 0.01% (w/v) to about 0.25% (w/v). It will be apparent to the skilled artisan that the time of incubation in a PAR ligand may vary according to cell type, and it well within the ken of a skilled addressee to determine such parameters without undue experimentation. For example, the time of incubation is sufficient for activation of one or more downstream cellular effects of the receptor to occur, as determined by routine procedures. It will also be apparent to the skilled artisan that the temperature for the incubation in PAR ligand is preferably between about 15° C. and about 37° C., or preferably room temperature, or more preferably 37° C.

For example, Ishii et al., J. Biol. Chem. 270 (27):16435-16440 (1995) describe a method for activating PAR using thrombin. Thrombin may be purchased from a variety of commercial sources, e.g., Sigma, and the final thrombin concentration is preferably in the range of about 10 nM to about 100 nM thrombin, including 10 nM thrombin, or 20 nM thrombin, or 30 nM thrombin, or 40 nM thrombin, or 50 nM thrombin, or 60 nM thrombin, or 70 nM thrombin, or 80 nM thrombin, or 90 nM thrombin, or 100 nM thrombin. Preferably, thrombin is diluted in phosphate-buffered saline optionally comprising about 0.5% (v/v) polyethylene glycol 8000. In one example, cells are incubated with thrombin at about 25° C. for about 60 min.

In another example, Quinton et al., J. Biol. Chem. 279 (18): 18434-18439 (2004) describe activation of PAR using plasmin.

In other examples, PAR can be activated by any one of the methods described by Shintani et al., British Journal of Pharmacology (2001) 133, 1276-1285 or Wang et al., Biochem. J. 408: 221-230 (2007), or Dery et al., Am. J. Physiol. 274 (Cell Physiol. 43): C1429-1452, incorporated herein by reference.

In another example, to activate any one of the PAR receptors the adherent cells are incubated in the presence of a known GPCR receptor agonist.

### 4. Storage of Cells

In a preferred example, the progenitor cells prepared according to the invention are stored in a suitable media conditions until required for differentiation. Preferably, where the differentiated cells are optionally exposed to prolonged incubation in low serum media, the progenitor cells prepared according to the invention are stored in low-serum medium until required for differentiation. Alternatively, the cells are stored in medium containing serum, e.g., DMEM-HG containing 10% FCS. Optionally, where the cells are further cultured, maintained or incubated under high density conditions the progenitor cells prepared according to the invention are preferably stored in a high density plating medium capable of supporting progenitor cells until further required.

Optionally, the cells are stored in low serum conditions at 4° C. for a short time. For example, the cells may be stored on ice for 1 min to 6 hours.

In one example, the cells are cryogenically frozen in liquid nitrogen. The method used to freeze the cells in optimal freezing media and conditions will be apparent to the skilled artisan and is dependent on the cell type. For example, such methods are commercially available from cell suppliers such as American Type Culture Collection (Rockville, Md.) or PromoCell® (Banksia Scientific Company, QLD). Methods that are used are also described in Animal Cell Culture: Practical Approach, Third Edition (John R.W. Masters, ed., 2000), ISBN 0199637970.

### 5. Differentiation

The present invention provides that the cells prepared according to the invention are differentiated into any other differentiated cell type. For example, a cell type of a tissue that is required for regeneration. The tissue may be a tissue of any part of the body including but not limited to organs such as skin, bone, gut, stomach, pancreas, thymus, thyroid, eye, spleen, heart, blood vessels, cardiovascular, blood, bone marrow, or any nervous tissue.

Preferably, the cells of the invention are differentiated to, but not limited to: cardiac tissue cells and/or skin cells and/or epidermal cells and/or keratinocytes and/or melanocytes and/or epithelial cells and/or dopaminogenic cells and/or neural cells such as those derived from the peripheral nervous system (PNS) and central nervous system (CNS) including but not limited to glial cells and/or Schwann cells and/or astrocytes and/or oligodendrocytes and/or microglial cells and/or and blood cells and/or such as lymphocytes, T cells and B cells, macrophages, monocytes and/or dendritic cells and/or Lagerhans cells and/or eosinophils and/or adipocytes and/or cardiomyocytes and/or cardiac muscle cells and/or cardiac fibroblasts and/or osteoclasts and/or osteoblasts and/or endocrine cells and/or β-islet cells of the pancreas and/or endothelial cells and/or epithelial cells and/or granulocytes and/or hair cells and/or mast cells and/or myoblasts and/or Sertoli cells and/or striated muscle cells and/or zymogenic cells and/or oxynitic cells and/or brush-border cells and/or goblet cells and/or hepatocytes and/or Kupffer cells and/or stratified squamous cells and/or pneumocytes and/or parietal cells and/or podocytes and/or synovial cells such as synovial fibroblasts and/or serosal cells and/or pericytes and/or chondrocytes and/or osteocytes and/or Purkinje fiber cells and/or myoepithelial cells and/or megakaryocytes, and the like and/or combinations thereof. In one preferred example, the differentiated cell produced by the method according to any example hereof is cardiac tissue cell, such as cardiac fibroblast.

Methods for differentiating cells of the invention include, but are not limited to the methods described in any one of the Examples or an example herein. The present invention also provides the differentiation of further cell types including, but not limited to the following.

### Neural Tissue Development

To differentiate cells prepared according to the invention, cells that have been de-differentiated according to any example as described herein are then suspended in Neuroblast A medium (Invitrogen/GIBCO) supplemented with 5% horse serum, 1% fetal calf serum, L-glutamine (2 mM), transferrin (100 μg/ml), insulin (2 μg/ml), retinoic acid 0.5 mM, brain-derived neurothrophic factor (10 ng/ml), and then allowed to attach, i.e. are plated onto tissue culture plates in said medium for a time sufficient to differentiate the cells to a neural phenotype.

### Dopamine-Secreting Issue Development

To differentiate cells prepared according to the invention, cells that have been de-differentiated according to any example as described herein are first suspended in dopaminergic induction media (DMEM serum free medium supplemented with 2 mM glutamine, 100 μg/ml streptomycin, 100 U/ml penicillin, 12.5 U/ml nystatin, N2 supplement (Invitrogen, New Haven, Conn.), and 20 ng/ml fibroblast growth factor-2 (FGF-2) and epidermal growth factor (EGF) (both from R&D Systems, Minneapolis, Minn.) for 2-3 days. The medium is then changed to basal induction medium containing Neurobasal and B27 (both from Invitrogen), in addition to 1 mM dibutyryl cyclic AMP (db cAMP), 3-isobutyl-1-methylxanthine (IBMX), and 200 μM ascorbic acid (all from Sigma, St Louis, Mo.) and brain-derived neurotrophic factor (BNDF) 50 ng/ml (Cytolab, Rehovot, Israel), as described in Barzilay et al., Stem cells and Development 17:547-554, 2008 which is herein incorporated by reference. The cells are then allowed to attach, i.e. are plated onto tissue culture plates in said medium for a time sufficient to differentiate the cells to a dopamine secreting phenotype.

### Skeletal/Cardiac Muscle Development

To differentiate cells prepared according to the invention, cells that have been de-differentiated according to any example as described herein are then suspended in alpha-Modification of Eagle's Medium supplemented with 10% fetal calf serum, L-glutamine (2 mM), ascorbate-2-phosphate (100 μM/ml), and 5-azacytodine (5 μM/ml) and then allowed to attach, i.e. are plated onto tissue culture plates in said medium for a time sufficient to differentiate the cells to a skeletal/muscle phenotype.

### Epithelial Development

To differentiate cells prepared according to the invention, cells that have been de-differentiated according to any example as described herein are then suspended in keratinocyte basal medium (Clonetics) supplemented with Bovine Pituitary Extract (50 μg/ml), epidermal growth factor (10 ng/ml), Hydrocortisone (0.5 μg/ml), Insulin (5 μg/ml) and then allowed to attach, i.e. are plated onto tissue culture plates in said medium for a time sufficient to differentiate the cells to a keratinocyte lineage.

### Osteoblasts, Tendon, Ligament or Odontoblast Development

To differentiate cells prepared according to the invention, cells that have been de-differentiated according to any example as described herein are then suspended in alpha-Modification of Eagle's Medium supplemented with 10% fetal calf serum, L-glutamine 2 mM, ascorbate-2-phosphate (100 μM), Dexamethasone (10⁻⁷M) and BMP-2 (50 ng/ml) and then allowed to attach, i.e. are plated onto tissue culture plates in said medium for a time sufficient to differentiate the cells.

### Pericyte or Smooth Muscle Cell Development

To differentiate cells prepared according to the invention, cells are suspended in alpha-Modification of Eagle's Medium supplemented with 10% fetal calf serum, L-glutamine 2 mM, ascorbate-2-phosphate (100 μM), platelet derived growth factor-BB (10 ng/ml) then layered over 200 μl of matrigel in 48-well plates for a time sufficient to differentiate the cells.

### Assessment of the Differentiated Phenotype

A method to assess the lineage of differentiated cells of the invention includes, but is not limited to use of commercially available antibodies and flow cytometry. This procedure has been reported previously and is well known in the art. Briefly, differentiated cell cultures are liberated by trypsin/EDTA digest then incubated for 30 min on ice. Approximately 2×10⁵ cells are washed then resuspended in 200 μl of primary antibody cocktail for 1 hr on ice. The primary antibody cocktail consists of saturating concentrations of a mouse IgG monoclonal antibody or rabbit IgG for each tube (Table 1). Antibodies for the markers listed in Table 1 are commercially available from a variety of sources including but not limited to DAKO, Santa Cruz, Pharmingen, or Sigma. For the staining with antibodies reactive with intracellular antigens the cells are first washed with PBS then permeablized by treatment with 70% ethanol on ice for ten minutes then washed prior to staining. The mouse isotype IgM and IgG negative control Mabs are treated under the same conditions. Following incubation with primary antibodies, cells are washed and exposed to saturating levels of goat anti-mouse IgM μ-chain specific-FITC (1/50 dilution) and either goat anti-mouse IgG γ-specific-PE (1/50 dilution) or anti-rabbit Ig-specific-PE (1/50 dilution) (Southern Biotechnology Associates) in a final volume of 100 μl. The cells are incubated for 45 min on ice, then washed twice then fixed in FAX FIX (PBS supplemented with 1% (v/v), 2% (w/v) D-glucose, 0.01% sodium azide). Flow cytometric analysis is performed using a FACSCalibur flow cytometer and the CellQuest software program (Becton Dickinson Immunocytometry Systems, San Jose, Calif.). Data analysis is performed using CellQuest and the Modfit LT V2.0 software program (Verity Software House, Topsham, Me.).

### Table 1: Markers for Lineage Identification

1. Skeletal Muscle: Myo E Desmin
2. Smooth Muscle: SMMHC, SMHC-FAST, alphaSMAC, PDGF-R, Vimentin;
3. Chondrocytes: Type II Collagen; Collagen IX; Aggrecan; Link Protein; S100; Biglycan;
4. Basal Fibroblasts: Laminin; Type IV Collagen; Versican;
5. Endothelial Cells: vWF; VCAM-1; Endoglin; MUC18; CD31; CD34; SDF-1
6. Cardiomyocytes: Calponin; Troponin I; Troponin C;
7. Neurons: NCAM; GFAP; Neuroanalase; Neurofilament;
8. Bone: AP, Type I Collagen; CBFA 1; OCN; OPG; RANKL; Annexin II
9. Fat: CEPBalpha; PPARgamma; Leptin;
10. Epithelial cells: Keratin 14; Cytokeratin 10+13; EGFR;
11. Fibroblast: Collagen III; NGFR; Fibroblast marker;
12. Haematopoietic: CD14; CD45; Glycophorin-A.

### 6. Formulations and Treatments

Pharmaceutical compositions and other formulations for application to the human or animal body e.g., for stimulating or enhancing tissue repair in a subject, are suitable for use topically, systemically, or locally as an injectable and/or transplant and/or device, usually by adding necessary buffers.

Preferred formulations for administration, the non-culture expanded cells used in this invention are in a pyrogen-free, physiologically acceptable form.

The cells may be injected in a viscous form for delivery to the site of tissue damage.

Topical administration may be suitable for wound healing and tissue repair.

In one example, therapeutically useful agents may also optionally be included in the progenitor cell formulation, or alternatively, administered simultaneously or sequentially with the composition in the methods of the invention.

In another example, the compositions of the present invention may be used in conjunction with presently available treatments for tendon/ligament injuries, such as suture (e.g., vicryl sutures or surgical gut sutures, Ethicon Inc., Somerville, N.J., USA) or tendon/ligament allograft or autograft, in order to enhance or accelerate the healing potential of the suture or graft.

The choice of a carrier material is based on biocompatibility, biodegradability, mechanical properties, cosmetic appearance and interface properties. The particular application of the progenitor cells will generally define the appropriate carrier. In one example, cells are mixed with a matrix, preferably a biodegradable matrix or a matrix comprised of pure proteins or extracellular matrix components. Other useful matrices include e.g., collagen-based materials including sponges, such as Helistat™ (Integra Life Sciences, Plainsboro, N.J., USA), or collagen in an injectable form, and sequestering agents such as hyalouronic acid-derived materials. Biodegradable materials, such as cellulose films, or surgical meshes, may also serve as matrices. Such matrices may be sutured into an injury site, or wrapped around a site of injury such as a tendon or ligament. Another preferred class of carriers includes polymeric matrices, wherein the progenitor cell of the invention is mixed with a polymer of poly lactic acid, poly glycolic acid, or a copolymer of lactic acid and glycolic acid. These matrices may be in the form of a sponge, or in the form of porous particles, and may also include a sequestering agent. Suitable polymer matrices are described, for example, in WO93/00050 which is incorporated herein by reference in its entirety.

In another example, the formulations of progenitor cells of the invention may comprise other therapeutically useful agents such as, for example, one or more cytokines, chemokines, leukemia inhibitory factor (LIF/HILDA/DIA), migration inhibition factor, MP52, growth factors including epidermal growth factor (EGF), fibroblast growth factor (FGF), platelet derived growth factor (PDGF), transforming growth factors (TGF-alpha and TGF-beta), and fibroblast growth factor-4 (FGF-4), parathyroid hormone (PTH), insulin-like growth factors (IGF-I and IGF-II), or combinations thereof.

In another example, the formulation comprises at least one other agent that promotes hematopoiesis, such as, for example a cytokine, which participates in hematopoiesis. Some non-limiting examples are: CSF-1, G-CSF, GM-CSF, interleukins, interferons, or combinations thereof.

In another example, the formulation comprises at least one other agent that promotes the delivery of systemic proteins such as Factor IX, VIII, growth hormone etc.

In another example, the progenitor cells are genetically engineered to express a protein of interest prior to the application to the subject in need. The protein of interest is any macromolecule, which is necessary for cell growth, morphogenesis, differentiation, tissue building or combinations thereof. These are, for example, a bone morphogenic protein, a bone morphogenic-like protein, an epidermal growth factor, a fibroblast growth factor, a platelet derived growth factor, an insulin like growth factor, a transforming growth factor, a vascular endothelial growth factor, cytokines related to hematopoiesis, factors for systemic delivery as such as GH, factor VIII, factor IX or combinations thereof.

The term “cells engineered to express a protein of interest” is defined hereinabove as a cell or to a tissue which had been modified via molecular biology techniques, for example via recombinant DNA technology, to express any macromolecule which is necessary for cell growth, morphogenesis, differentiation, tissue building or combinations thereof. In another example, cells are thus modified in order to produce an increased amount of any macromolecule, which is necessary for cell growth, morphogenesis, differentiation, tissue building or combinations thereof.

The step of genetically engineered a cell to express a protein of interest is performed by the transfection or transduction of the cell with a nucleic acid encoding the protein of interest.

The term “transfection” or “transfected cells” refer to cells in which DNA is integrated into the genome by a method of transfection, i.e. by the use of plasmids or liposomes.

The term “transduction” or “transduced cells” refers to viral DNA transfer for example, by phage or retroviruses. The nucleic acid, which encodes the protein of interest, can be introduced by a vector molecule, as well, and represents an additional example of this invention.

The vector molecule can be any molecule capable of being delivered and maintained, within the target cell, or tissue such that the gene encoding the product of interest can be stably expressed. In one example, the vector utilized in the present invention is a viral or retroviral vector or a non-viral DNA plasmid. According to one aspect, the method includes introducing the gene encoding the product into the cell of the mammalian tissue for a therapeutic or prophylactic use. The viral vectors, used in the methods of the present invention, can be selected from the group consisting of (a) a retroviral vector, such as MFG or pLJ; (b) an adeno-associated virus; (c) an adenovirus; and (d) a herpes virus, including but not limited to herpes simplex 1 or herpes simples 2; (e) lentivirus and any combination of (a) to (e). Alternatively, a non-viral vector, such as a DNA plasmid vector, can be used. Any DNA plasmid vector known to one of ordinary skill in the art capable of stable maintenance, within the targeted cell, or tissue upon delivery, regardless of the method of delivery utilized is within the scope of the present invention. Non-viral means for introducing the gene encoding for the product into the target cell are also within the scope of the present invention. Such non-viral means can be selected from the group consisting of (a) at least one liposome, (b) Ca₃(PO₄)₂, (c) electroporation, (d) DEAE-dextran, (e) injection of naked DNA, and any combination of (a) to (e).

The term “nucleic acid” refers to polynucleotides or to oligonucleotides such as deoxyribonucleic acid (DNA), and, where appropriate, ribonucleic acid (RNA) or mimetics thereof. The term should also be understood to include, as equivalents, analogs of either RNA or DNA made from nucleotide analogs, and, as applicable to the example being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, enhanced cellular uptake, enhanced affinity for nucleic acid target and increased stability in the presence of nucleases.

The formulations of the invention are useful for treating cartilaginous tissue, defects of the embryonic joint where tendon, ligaments, and bone form simultaneously at contiguous anatomical locations, regenerating tissue at the site of tendon attachment to bone, or for wound healing, such as skin healing and related tissue repair. Types of wounds include, but are not limited to burns, incisions and ulcers.

The formulations of the invention are also useful for tissue renewal or regeneration that ameliorates an adverse condition of tissue, degeneration, depletion or damage such as might be caused by aging, genetic or infectious disease, accident or any other cause, in humans, livestock, domestic animals or any other animal species.

In another example the formulations of the invention are also useful for promoting tissue development in livestock, domestic animals or any other animal species in order to achieve increased growth for commercial or any other purpose.

In another example the formulations of the invention are also useful in plastic surgeries, such as, for example, facial or body reconstruction.

In another example the formulations of the invention are also useful for enhancing repair of tissue injuries, tears, deformities or defects, and for the prophylaxis or prevention of tissue damage.

In another example, the formulations of the invention are also useful for treating and/or preventing osteoporosis, which results from a decrease in estrogen, which may be caused by menopause or ovariectomy in women. Use of the progenitor cells of the present invention for prevention of accelerated bone resorption and inhibition of a decrease of bone volume, bone quality and bone strength is also provided by the invention. Trabecular connectivity and trabecular unconnectivity may be maintained at healthy levels with the pharmaceutical compositions of the present invention. Osteoporosis and its symptoms such as decreased bone volume, bone quality, and bone strength, decreased trabecular connectivity, and increased trabecular unconnectivity may be treated or prevented by administration of a pharmaceutically effective amount of the pharmaceutical composition to a patient in need thereof.

In another example, the formulations of the invention are also useful for regenerating tissues which have been damaged through acute injury, abnormal genetic expression or acquired disease. In one such example, the formulations of the invention are useful for regenerating cardiac tissue such as a cardiac muscle tissue.

In another example, the formulations of the invention are also useful for stimulating skeletal development in livestock, domestic animals or any other animal species in order to achieve increased growth for commercial or any other purpose.

In another example, the formulations of the invention are also useful for treatment of neoplasia or hyperplasia of bone or cartilage or any other tissue, in humans, livestock, domestic animals or any other animal species.

In another example, the formulations of the invention are also useful for stimulating haematopoiesis e.g., in combination with hematopoietic transplants.

In another example, the formulations of the invention are also useful for treatment and/or prevention of cancer, for example by stimulating and/or replenishing growth of health cells in a tumour site.

The dosage regimen, which is the amount of the cells that are administered in order to obtain a therapeutic effect, is affected by various factors which modify the action of the progenitor cells' composition, e.g., amount of tissue desired to be repaired or formed, the site of injury or damage, the condition of the damaged tissue, the size of a wound, type of damaged tissue, the patient's age, sex, and diet, the severity of any infection, time of administration and other clinical factors. The dosage may vary with the type of matrix used in the reconstitution and the types of additional proteins in the composition. The addition of other known growth factors, such as IGF-I (insulin like growth factor I), to the final composition, may also affect the dosage. Progress can be monitored by periodic assessment of tissue formation and/or growth and/or repair. The progress can be monitored by methods known in the art, for example, X-rays (CT), ultra-sound, MM, arthroscopy and histomorphometric determinations.

### 7. Tissue and Organ Building, Repair and Regeneration

The present invention encompasses the use of the progenitor cells prepared according to the invention or differentiated cells derived there from for building, repairing or regenerating a tissue, and/or building, repairing or regenerating an organ. It is apparent that when progenitor cells are used in this example, those progenitor cells differentiate in situ during the tissue/organ building, repair or regeneration, whereas differentiated cells derived from the progenitor cells are not required to differentiate in situ.

It is also apparent that when differentiated cells are employed in this example, multiple cell types may be required to build, regenerate or repair tissues comprising different cell types in nature, or whole organs. One or more, or all, of these different cell types may be produced in accordance with the present invention by employing appropriate differentiation media and conditions. A plurality of progenitor cell populations may each be derived from different starting cells or cell types, or produced in different batches. Similarly, a plurality of differentiated cells may comprise different batches of the same cell type and/or different cell types per se produced from the same or different batches of progenitor cells or the same or different starting cell types.

The organ that is produced, repaired or regenerated is without limitation and includes e.g., skin, bone, gut, stomach, pancreas, thymus, thyroid, eye, spleen, heart, blood vessels, cardiovascular, bone marrow, or nervous system, a cardiovascular organ such as artery, or combinations thereof. The tissue may be any tissue without limitation including e.g., a tissue of any one or more of the foregoing organs and further includes skin, muscle, fat, bone, or any tissue derived from the group of endoderm, mesoderm, ectoderm or combination thereof and including cartilage, connective tissue, tendon, nerve adipose, gastrointestinal tissue, cardiac tissue such as of the heart, cornea, optical tissue, exocrine and/or endocrine glands. For example if subcutaneous fatty tissue were to be regenerated it would include the regeneration of the primary cell type i.e. fat, and its blood supply (vascular tissue) nerve supply and stromal tissue (supporting structures including ECM, basil lamina etc). Similarly this concept can be used to support the regeneration of most tissues e.g. for muscle it will be myocytes, vascular supply and nerve supply and stromal tissue.

In one example, the tissue and/or organ to be regenerated may be tissue and/or organ injured, lost, or atrophied by disease processes or degeneration. Such tissues and/or organs could be the spinal cord (for example, multiple sclerosis), the substantia nigra in Parkinson's disease, or the olfactory mucosa or Alzheimer's disease, a cardiac muscle or cardiovascular organ such as heart such as after myocardial infraction. It will be understood that progenitor cells of the present invention may be provided to individuals predisposed to any condition resulting in tissue and/or organ loss, injured or atrophied such as multiple sclerosis, Parkinson's or Alzheimer's disease, cancer, cardiac injury such as myocardial infraction or to individuals having symptoms of onset of these diseases for preventing or reducing the severity of these diseases.

In one example, the progenitor cells prepared according to any example hereof are used to build, repair or regenerate a tissue and/or organ or an element of a tissue and/or organ e.g., in situ at a site of injury to a tissue and/or organ. In one example, such regeneration is achieved by providing the progenitor cells at least one of a neuropeptide Y (NPY), a fragment or variant of neuropeptide Y, a compound capable of inducing expression of a gene encoding a neuropeptide Y protein or fragment or variant thereof, a cell that produces a neuropeptide Y and/or an agonist or antagonist of a neuropeptide Y receptor to induce building, repair or regeneration of a tissue and/or organ e.g., at the site of injury, as described in International Application PCT/AU2006/000481 filed Apr. 10, 2006 (Publication No. WO/2006/108218) which is incorporated herein by reference in its entirety. In one preferred example, the fragment or variant of neuropeptide Y is biologically functional. The progenitor cells may be provided or administered directly to a site of injury in the tissue and/or organ. Alternatively, or in addition, the progenitor cells are produced in situ as described according to any example hereof.

Alternatively, or in addition, progenitor cells prepared in accordance with any example hereof are used to build, repair or regenerate a tissue or organ or an element of a tissue and/or organ e.g., in situ at a site of injury in a tissue and/or organ. In one example, such regeneration is achieved by providing the progenitor cells at least one of a neuregulin, a fragment of a neuregulin, a compound capable of inducing expression of a neuregulin gene, and/or an agonist or antagonist of a receptor for neuregulin to induce building, repair or regeneration of a tissue or organ e.g., at the site of injury, substantially as described in International Application PCT/AU2007/000238 filed Feb. 28, 2007 (Publication No. WO/2007/098541) which is incorporated herein by reference in it entirety. In one preferred example, the fragment of neuregulin is biologically functional. The progenitor cells may be provided or administered directly to a site of injury in the tissue and/or organ. Alternatively, or in addition, the progenitor cells are produced in situ as described according to any example hereof.

Alternatively, or in addition, the progenitor cells prepared by the methods according to any example hereof are used to build, repair or regenerate a tissue and/or organ or an element of a tissue and/or organ e.g., in situ at a site of injury in the tissue or organ. In one example, such regeneration is achieved by providing the progenitor cells at least one of a neurotrophin, a fragment of a neurotrophin, a compound capable of inducing expression of a neurotrophin gene, and/or an agonist or antagonist of a receptor for a neurotrophin to induce building, repair or regeneration of a tissue or organ e.g., at the site of injury, substantially as described in International Application PCT/AU2007/000238 filed Feb. 28, 2007 (Publication No. WO/2007/098541) which is incorporated herein by reference in it entirety. Non-limiting examples of neurotrophin(s) suitable for use in the present invention include nerve growth factor (NGF), neurotrophic factor 3 (NT-3), brain derived neurotrophic factor (BDNF), neurotrophic factor 4 (NT-4), neurotrophic factor 5 (NT-5) or Ciliary Neurotrophic Factor CNTF. In a particularly preferred example, the neurotrophin is NGF. In one example, the fragment of neurotrophin is biologically functional. The progenitor cells may be provided or administered directly to a site of injury in the tissue and/or organ. Alternatively, or in addition, the progenitor cells are produced in situ as described according to any example hereof.

In one example, one or more populations (or batches) of progenitor cells or one or more populations of differentiated cells derived from the progenitor cells as described according to any example hereof is cultured or perfused onto a scaffold or matrix that allows the cells to develop into a tissue or organ or part thereof e.g., a biocompatible scaffold or matrix such as a biodegradable scaffold matrix.

In another example, building or regenerating an organ or multi-layered tissue such as an artificial organ or tissue may be achieved by a process comprising:
(i) perfusing a first population of progenitor cells produced in according with any example hereof or differentiated cells derived therefrom into and/or onto a first side of a biocompatible scaffold or matrix such that the cells attach to the matrix and then culturing the cells for a time and under conditions sufficient to produce a first specialized tissue layer; and
(ii) perfusing a second population of undifferentiated or differentiated cells distinct from the cells at (i) into and/or onto a second side of the biocompatible matrix such that the second population of cells attaches to the matrix and then culturing the second population of cells in the matrix for a time and under conditions sufficient to produce a second specialized tissue layer that is different from the first specialized tissue layer
   to thereby create a multi-layered tissue and/or organ construct.

This process may be achieved by reversing the order of (i) and (ii).

In another example, building or regenerating an organ or multi-layered tissue such as an artificial organ or tissue may be achieved by a process comprising:
(i) perfusing a first population of progenitor cells produced in according with any example hereof or differentiated cells derived therefrom into and/or onto a first side of a biocompatible scaffold or matrix such that the cells attach to the matrix and then culturing the cells for a time and under conditions sufficient to produce a first specialized tissue layer; and
(ii) perfusing a second population of progenitor cells produced in according with any example hereof or differentiated cells derived therefrom into and/or onto a second side of the biocompatible matrix such that the second population of cells attaches to the matrix and then culturing the second population of cells in the matrix for a time and under conditions sufficient to produce a second specialized tissue layer that is different from the first specialized tissue layer
   to thereby create a multi-layered tissue and/or organ construct.

In another example, a multi-layered tissue and/or organ construct can also be created by culturing first and second populations of cells on the same side of the biocompatible matrix.

In another example, different populations of cells are cultured simultaneously or sequentially in and/or on the matrix.

In accordance with these examples, perfused cells are cultured until they differentiate and/or proliferate to produce a first monolayer comprising cells with a desired phenotype and morphology. Once the first monolayer has attained a desired cell density, a second layer of the same cell population is deposited on the first monolayer. The second layer of perfused cells is cultured under conditions to provide nutrients to both the second cell layer and the first monolayer and for time sufficient for cells in the layers to form a bilayer having cells with a desired cell type and morphology. The process is repeated until a poly-layer comprising a plurality of cell monolayers of the desired cell type and morphology is produced. Polylayers may also be produced by layering of multiple bilayers, trilayers, etc.

In another example, the invention provides a tissue construct or organ construct comprising a biocompatible scaffold or matrix perfused with at least one population of progenitor cells of the present invention and/or one or more populations of differentiated cells derived from progenitor cells of the invention. The tissue or organ construct may comprise one or a plurality of cell types or populations or batches e.g., a plurality of cell types on the same or different sides of the biocompatible scaffold or matrix.

As used herein, the term “scaffold” or “matrix” shall be taken to mean any material in and/or on which cells may differentiate and/or proliferate to form a tissue or organ or part thereof. Accordingly, a scaffold or matrix provides the structure or outline to the tissue or organ to be repaired, regenerated or built. A scaffold or matrix will generally be a three-dimensional structure comprising a non-degradable or a biodegradable material, e.g., a decellularized organ or part thereof that can be shaped into a desired tissue or organ. For example, a scaffold or matrix also provides sufficient interstitial distances required for cell-cell interaction.

As used herein, the term “biocompatible scaffold” or “biocompatible matrix” shall be taken to mean a scaffold or matrix as hereinbefore defined that, with any tissue and/or organ proliferating or growing thereon, is further suitable for implantation into a host subject. When grown in a biocompatible matrix, the proliferating cells mature and segregate properly to form tissues analogous to counterparts found in vivo. In other examples, counter parts tissues or organs present in vivo may be replaced by a tissue and/or organ repaired, regenerated or repaired by the method described herein.

A biocompatible scaffold or matrix is generally a polymeric composition e.g., polyglycolic acid, or the infra-structure of an organ following decellularization i.e., removal of substantially all cellular material. Non-limiting examples of biocompatible polymeric matrixes can be formed from materials selected from, but are not limited to cellulose ether and/or cellulose and/or cellulosic ester and/or fluorinated polyethylene and/or poly-4-methylpentene and/or polyacrylonitrile and/or polyamide and/or polyamideimide and/or polyacrylate and/or polybenzoxazole and/or polycarbonate and/or polycyanoarylether and/or polyester and/or polyestercarbonate and/or polyether and/or polyetheretherketone and/or polyetherimide and/or polyetherketone and/or polyethersulfone and/or polyethylene and/or polyfluoroolefin and/or polyglycolic acid and/or polyimide and/or polyolefin and/or polyoxadiazole and/or polyphenylene oxide and/or polyphenylene sulfide and/or polypropylene and/or polystyrene and/or polysulfide and/or polysulfone and/or polytetrafluoroethylene and/or polythioether and/or polytriazole and/or polyurethane and/or polyvinyl and/or polyvinylidene fluoride and/or regenerated cellulose and/or silicone and/or urea-formaldehyde and/or copolymers or physical blends thereof, and any combination thereof. The polymeric matrix can be coated with a biocompatible and biodegradable shaped setting material. In one example, the shape settling material is a liquid copolymer e.g., poly-DL-lactide-co-glycolide. In another example, the scaffold or matrix comprises synthetic or semi-synthetic polymer fibers e.g., Dacron™, Teflon™ or Gore-Tex™.

Preferred non-toxic biocompatible scaffolds or matrices may be made of natural or synthetic polymers, such as, for example, collagen, poly(alpha esters) such as poly(lactate acid), poly(glycolic acid) (PGA), polyorthoesters and polyanhydrides and their copolymers, which degraded by hydrolysis at a controlled rate and are reabsorbed. These materials provide the maximum control of degradability, manageability, size and configuration. Preferred biodegradable polymer material includes polyglycolic acid and polygalactin, developed as absorbable synthetic suture material. Polyglycolic acid and polygalactin fibers may be used as supplied by the manufacturer. Other biodegradable materials include cellulose ether and/or cellulose and/or cellulosic ester and/or fluorinated polyethylene and/or phenolic polymer and/or poly-4-methylpentene and/or polyacrylonitrile and/or polyamide, polyamideimide and/or polyacrylate and/or polybenzoxazole and/or polycarbonate and/or polycyanoarylether and/or polyester and/or polyestercarbonate and/or polyether and/or polyetheretherketone and/or polyetherimide and/or polyetherketone and/or polyethersulfone and/or polyethylene and/or polyfluoroolefin and/or polyimide and/or polyolefin and/or polyoxadiazole and/or polyphenylene oxide and/or polyphenylene sulfide and/or polypropylene and/or polystyrene and/or polysulfide and/or polysulfone and/or polytetrafluoroethylene and/or polythioether and/or polytriazole and/or polyurethane and/or polyvinyl and/or polyvinylidene fluoride and/or regenerated cellulose and/or silicone and/or urea-formaldehyde and/or or copolymers or physical blends of these materials and any combination thereof.

Decellularized scaffolds or matrices are produced by a process in which the entire cellular and tissue content is removed, leaving behind a complex infra-structure e.g., comprising a fibrous network of stroma or unspecialized connective tissue that predominantly comprises collagen and/or proteoglycan. Decellularized structures can be rigid or semi-rigid. Methods of producing decellularized matrix or scaffold are described e.g., in U.S. Pat. No. 7,354,702 and U.S. Pat. No. 7,429,490, both of which are incorporated herein by reference in their entirety.

Scaffolds or matrices may be impregnated with suitable antimicrobial agents and may be colored by a color additive to improve visibility and to aid in surgical procedures.

In one preferred example, the biocompatible polymer is a synthetic absorbable polygalactin material or polyglycolic acid (PGA) fibers (Ethicon Co., Somerville, N.J.; Craig P. H., et al. Surg. 141; 1010 (1975) or Christenson L, et al., Tissue Eng. 3 (1): 71-73; discussion 73-76 (1997)) which can be used as supplied by the manufacturer. This biocompatible polymer may be shaped using methods such as, for example, solvent casting, compression moulding, suturing, filament drawing, meshing, leaching, weaving and coating (See Mikos, U.S. Pat. No. 5,514,378, hereby incorporated by reference in its entirety).

In some examples, the polymers are coated with compounds such as basement membrane components, agar, agarose; gelatin, gum arabic, collagens, such as collagen types I, II, III, IV, and V, fibronectin, laminin, glycosaminoglycans, mixtures thereof, and other hydrophilic and peptide attachment materials having properties similar to biological matrix molecules known to those skilled in the art of cell culture.

Factors, including nutrients, growth factors, inducers of differentiation or dedifferentiation, products of secretion, immunomodulators, inhibitors of inflammation, regression factors, biologically active compounds which enhance or allow ingrowth of the lymphatic network or nerve fibers, and drugs, can be incorporated into the matrix or provided in conjunction with the matrix. Similarly, polymers comprising peptides such as the attachment peptide RGD (Arg-Gly-Asp) can be synthesized for use in forming matrices. Angiogenesis factors, cytokines, extracellular matrix components, and other bioactive materials or drugs may also be impregnated into the scaffold or matrix at any stage preceding implantation e.g., to promote repair, grafting, or reduce or inhibit rejection. Growth factors include e.g., epidermal growth factor (EGF), vascular endothelial growth factor (VEGF), heparin-binding epidermal-like growth factor (HBGF), fibroblast growth factor (FGF), cytokines, genes, proteins, and the like. Other useful additives include antibacterial and antifungal agents to promote healing by suppression of infections. For example, the biocompatible matrix can be fabricated to have a controlled pore structure that allows such nutrients to permeate or contact the perfused cells in the absence of significant cell migration through the pores. In vitro cell attachment and cell viability can be assessed using scanning electron microscopy, histology and quantitative assessment with radioisotopes.

In another example, additional collagenous layers may be added to the inner surfaces of the decellularized structure to create a smooth surface as described in International PCT Publication No. WO 95/22301, the contents of which are incorporated herein by reference. This smooth collagenous layer promotes cell attachment which facilitates growth and development. As described in International PCT Publication No WO 95/22301, this smooth collagenous layer may be made from acid-extracted fibrillar or non-fibrillar collagen, which is predominantly type I collagen, but may also include type II collagen, type IV collagen, or both. The collagen used may be derived from any number of mammalian sources, typically pig and cow skin and tendons. The collagen for example has been processed by acid extraction to result in a fibril dispersion or gel of high purity. Collagen may be acid-extracted from the collagen source using a weak acid, such as acetic, citric, or formic acid. Once extracted into solution, the collagen can be salt-precipitated using NaCl and recovered, using standard techniques such as centrifugation or filtration. Details of acid extracted collagen are described, for example, in U.S. Pat. No. 5,106,949 issued to Kemp et al., incorporated herein by reference in its entirety.

The present invention will now be illustrated by the following Examples, which are not intended to be limiting in any way. The teachings of all references cited herein are incorporated herein by reference.

### Example 1

### Preparation of Cells Having the Ability to Differentiate into Other Cell Types by Incubation in Medium Containing a Modulator of Src Pathway with or without Treatment with Protease: Method 1

This example describes how to produce the progenitor cells of the invention having an ability to differentiate into different cell types, by incubating differentiated cells e.g., fibroblasts in a medium containing one or more modulators of SRC e.g., FGF-1 or HGF or NGFβ or IL-1β or NT3 or with SEMA-3A to agonise or partially agonise the SRC pathway. Cells are then either incubated in a medium comprising protease e.g., trypsin or without protease. The cells produced by this method can then be tested for their ability to differentiate into adipocytes, as determined by the accumulation of fat. Differentiation into adipocytes is selected in these primary experiments because methods for such differentiation are well-established.

### 1.1 Materials and Methods

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area.

Once all cells are attached, the medium is replaced with growth medium Dulbecco's Modified Eagle Medium Low Glucose (DMEM-LG) containing 0-3 mM glucose supplemented with about 0.01 μM to about 100 μM, preferably 1 μM to about 10 μM recombinant human FGF-1 (purchased from Sigma-Aldrich or R&D Systems, Minneapolis, Minn.) from about 5 minutes to about 48 hours. Control cells are incubated with the same medium as test cells without FGF-1.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM recombinant human HGF (purchased from R&D systems) for about 5 min to about 24 hours. Control cells are incubated with the same medium as test cells without HGF.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM recombinant NGF (purchased from Boehringer Mannheim, Germany) for about 5 min to about 24 hours. Control cells are incubated with the same medium as test cells without NGFβ.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM rat recombinant IL-1β (Sigma, St. Louis, Mo.) for about 5 min to about 24 hours. Control cells are incubated with the same medium as test cells without IL-1β.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM NT3 (purchased from Regeneron Pharmaceuticals (Tarryton, N.Y.) for about 5 min to about 24 hours. Control cells are incubated with the same medium as test cells without NT3.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM recombinant SEMA-3A (purchased from R&D systems, Minneapolis, Minn.) for about 5 min to about 24 hours. Control cells are incubated with the same medium as test cells without SEMA-3A.

At the conclusion of the incubation period in media containing FGF-1, HGF, NGFβ, IL-1β, NT3, or SEMA-3A cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Alternatively test cells are not detached, and are used directly in the differentiation assay as described below.

Differentiation into Adipocytes

Cells at a density of about 20,000 cells per well or about 740.74 cells per mm² surface area of the well are incubated in adipogenic differentiation medium (Medium 199 containing 170 nM insulin, 0.5 mM 3-isobutyl-1-methylxanthine, 0.2 mM indomethacin, 1 μM dexamethasone, and 15% rabbit serum) for about 12-21 days. Adipogenic media is replaced every 3 days on both test and control cells.

### Assessment of Adipogenesis

After incubation for about 12-21 days in adipogenic medium, the medium is removed, and cells are fixed in 10% formaldehyde solution in aqueous phosphate buffer for at least 1 hour. Cells are then washed with 60% isopropanol and stained with a working solution of Oil Red 0 solution (in 60% isopropanol, see below for preparation) for 10 minutes. The cells are then washed several times with water, and destained in 100% isopropanol for 15 minutes. The destain solution is removed and the optical density of the solution is measured at 500-510 nm.

The working solution of Oil red 0 is prepared as previously described (Humason 1972) by dissolving 4.2 g of Oil red 0 in 1200 ml absolute isopropanol and left overnight without stirring at room temperature. The solution is filtered through analytical filter paper 589-WH (Schleicher and Schuell); after filtration, 900 ml of distilled water is added and the solution is left overnight at 4° C. without stirring and subsequently filtered twice. This working solution is stored at room temperature and had a shelf life of 6-8 months.

For example, differentiation of the cell product into other cell types is achieved by reseeding the cells described above into differentiation media for adipocytes, or as described herein e.g., Examples 12 to 15.

### Example 2

### Preparation of Cells Having the Ability to Differentiate into Other Cell Types by Incubation in Medium Containing a Modulator of Src Pathway with or without Treatment with Protease: Method 2

This example describes how to produce the progenitor cells of the invention having an ability to differentiate into different cell types, by incubating differentiated cells e.g., fibroblasts in a medium containing one or more modulators of SRC e.g., PP1, PP2, or SU6656 to antagonise or partially antagonise the SRC pathway. Cells are then either incubated in a medium comprising protease e.g., trypsin or without protease. The cells produced by this method are then tested for their ability to differentiate into adipocytes, as determined by the accumulation of fat.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area.

Once all cells are attached, the medium is replaced with growth medium Dulbecco's Modified Eagle Medium Low Glucose (DMEM-LG) containing 0-3 mM glucose supplemented with about 0.01 μM to about 100 μM PP1 (4-amino-5-(4-methylphenyl)-7-(t-butyl)pyrazolo (3,4-d) pyrimidine) (purchased from Biomol, Plymouth Meeting, Pa.) for about 5 min to about 24 hours, preferably for about 1 hour. Control cells are incubated with the same medium as test cells without PP1.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM PP2 (4-Amino-5-(4-chlorophenyl)-7-(t-butyl)pyrazolo[3,4-d]pyrimidine) (purchased from Calibiochem, La Jolla, Calif.) for about 5 min to about 24 hours, preferably for about 1 hour. Control cells are incubated with the same medium as test cells without PP2.

Alternatively, once all cells are attached, the medium is replaced with growth medium Dulbecco's Modified Eagle Medium Low Glucose (DMEM-LG) containing 0-3 mM glucose supplemented with about 0.01 μM to about 100 μM SU6656 (purchased from EMD Biosciences, La Jolla, Calif.) dissolved in DMSO (Sigma) for about 5 min to about 24 hours, preferably for about 1 hour. Control cells are incubated with the same medium as test cells without SU6656.

At the conclusion of the incubation period in media containing PP1, PP2 or SU6656, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 p. 1 of detachment solution as described in Example 1 and incubated at 37° C. until cells lifted from the plates. Test cells are recovered from culture, then diluted to 200 p. 1 with DMEM-HG (e.g., Lonza, Cat #12-604) (with 10% FBS) and maintained in this medium until required for re-differentiation. Alternatively cells are not detached, and are used directly in the differentiation assay as described in Example 1. Differentiation into adipocytes and assessment of adipogenesis is carried out as before.

For example, differentiation of the cell product into other cell types is achieved by reseeding the cells into differentiation media for adipocytes as described in Example 1, or as described herein e.g., Examples 12 to 15.

### Example 3

### Preparation of Cells Having the Ability to Differentiate into Other Cell Types by Incubation in Medium Containing a Modulator of Src Pathway and Treatment with Protease, with Additional Incubation in Low-Serum: Method 3

This example describes how to produce the progenitor cells of the invention by incubating differentiated cells in medium containing one or more other modulators SRC pathway, and treating the cells with protease e.g., trypsin, and incubating cells in medium having low-serum concentration e.g., for 5-9 days. This method may provide an increase in the proportion of cells achieving optimum plasticity compared to the treatment with one or other modulators of SRC pathway alone.

Production of Cells Capable of Differentiating into Other Cell Types

Fresh adult human dermal fibroblasts described in Example 1 are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells were used as control cells, and the second set of cells were used for testing the capability of cells produced by the method to differentiate into adipocytes. Control cells are plated directly onto 96 well plates at about 20,000 cells per well or about 740.74 cells per 10 mm² surface area. Test cells are plated onto larger plates but at the same concentration of cells per mm² plating surface area of the vessel. Once all cells are attached, the medium is replaced with medium 199 (M199) (e.g., Sigma) supplemented with 0-1% FBS (low-serum) for different periods of time, from 1 to 11 days.

The medium is then replaced with DMEM-LG containing 0-3 mM glucose supplemented about 0.01 μM to about 100 μM, preferably 1 μM to about 10 μM recombinant human FGF-1 from about 5 minutes to about 48 hours and incubated as described in Example 1. Control cells are incubated with the same medium as test cells but without FGF-1. Alternatively, the medium is replaced with DMEM-HG supplemented with 0.01 μM to about 100 μM recombinant human HGF for about 5 min to about 24 hours and incubated as described in Example 1. Control cells are incubated with the same medium as test cells without HGF. Alternatively, the medium is replaced with DMEM-LG containing 0-3 mM glucose supplemented with about 0.01 μM to about 100 μM NGFβ for about 5 min to about 24 hours and incubated as described in Example 1. Control cells are incubated with the same medium as test cells without NGFβ. Alternatively, the medium is replaced with DMEM-LG containing 0-3 mM glucose supplemented with about 0.01 μM to about 100 μM rat recombinant IL-1β for about 5 min to about 24 hours, and incubated as described in Example 1. Control cells are incubated with the same medium as test cells without IL-1β. Alternatively, the medium is replaced with DMEM-HG containing supplemented with about 0.01 μM to about 100 μM NT3 for about 5 min to about 24 hours, and incubated as described in Example 1. Control cells are incubated with the same medium as test cells without NT3. Alternatively, the medium is replaced with DMEM-HG supplemented with about 0.01 μM to about 100 μM SEMA-3A for about 5 min to about 24 hours, and incubated as described in Example 1. Control cells are incubated with the same medium as test cells without SEMA-3.

At the conclusion of the incubation period in low serum media, test cells were detached by the addition of 20 μl of detachment solution as in Example 1 and incubated at 37° C. until cells lifted from the plates. Test cells were recovered from culture, then diluted to 200 μl with serum-free DMEM-HG (0% FBS) and maintained in serum-free medium until required for re-differentiation. Control cells are not detached, and are used directly in the differentiation assay as described in Example 1. Differentiation into Adipocytes and assessment of adipogenesis is carried out as described in Example 1. The person skilled in the art would appreciate that differentiation of test cells into adipocytes may continue albeit at below optimum even after the 11-day period incubation at low serum.

For example, differentiation of the cell product into other cell types is achieved by reseeding the cells described into differentiation media for adipocytes in Example 1, or as described herein e.g., Examples 12 to 15.

### Example 4

### Preparation of Cells Having the Ability to Differentiate into Other Cell Types by Incubation with a Modulator of Src Pathway and Treatment with Protease and Additional Incubation at High Cell Density Conditions: Method 4

This example describes how to produce the progenitor cells of the invention by incubating differentiated cells e.g., fibroblasts in a medium containing one or more modulators of SRC pathway and treating the cells with protease, and incubating cells at high density conditions. This method may provide an increase in the proportion of cells achieving optimum plasticity compared to the treatment with one or more modulators of SCR pathway with or without protease treatment pathways alone.

In this set of experiments for producing cells having the ability to differentiate into different cell types, fresh human dermal fibroblasts derived from adult skin or from foreskin fibroblasts are cultured and detached by incubation with trypsin essentially as described in examples 1 to 3.

Test cells are then recovered from culture immediately after trypsinization and are diluted to about 100,000 cells in 100 μl in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and within about 4 to 6 hours after trypsinization, the recovered cells are seeded at concentrations of about 100,000 cells per well/plate or at about 3703.7 cells per mm2 surface area of the well/plate before attachment of the cells to the plate/well directly in 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum) for a time and under conditions sufficient for an optimum number of progenitor cells to be produced e.g., for up to about 24 hours or until adherence is achieved i.e., a shorter time than required for cells to become adherent and/or as determined by analysis of cell marker expression and/or by the ability of aliquots of cells to subsequently undergo differentiation.

As a negative control for the production of progenitor cells, trypsinized cells are seeded at a reduced density i.e., about 740.1 cells per mm² surface area, in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and incubated as for samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation.

Differentiation into Adipocytes

For differentiation into adipocytes, cells are incubated in radiogenic medium (Medium 199 containing 170 nM insulin, 0.5 mM 3-isobutyl-1-methylxanthine, 0.2 mM indomethacin, 1 μM dexamethasone, and 15% rabbit serum) at high density and allowed to expand for about 10-21 days.

As a negative control for differentiation, trypsinized cells are seeded at high density in high density plating medium (e.g., DMEM-HG supplemented with 10% FBS) and incubated as for test samples seeded at high density e.g., for up to about 24 hours or until adherent and/or as determined by analysis of cell marker expression and/or by the ability of aliquots of cells to subsequently undergo differentiation. The high density plating medium is then replaced with 200 to 400 μl DMEM-HG (10% FCS) medium and cells are allowed to expand for about 10-21 days.

As positive control for differentiation, rat bone marrow stoma/stem cells (brisks) are expanded in DMEM medium containing L-Glutamine and 10% FBS, and allowed to attach and reach sub-confluence or confluence. These cells are then detached by incubation with try sin as described above, and seeded at concentration of about 50,000 cells per well/plate or at about 1851.9 cells per mm² surface area of the well/plate in 400 μl DMEM-HG containing 10% FBS for up to about hours or until adherent. The medium is replaced from adherent culture with 200 to 400 adipogenic medium (Medium 199 containing 170 nM insulin, 0.5 mM 3-isobutyl-1-methylxanthine, 0.2 mM indomethacin, 1 μM dexamethasone, and 15% rabbit serum) and cells are allowed to expand for about 10-21 days.

Medium is replaced every 3 days for both test cells and negative and positive control cells.

### Assessment of Adipogenesis

After incubation for 12-21 days in adipogenic medium, differentiation potential of test cells compared to control cells at each day of incubation at high density post incubation optionally with low-serum and trypsinization is measured by an assessment of adipogenisis as described in example 1.

For example, differentiation of the cell product into different cell types is achieved by reseeding the cells into differentiation media for adipocytes as described in this example, or as described herein e.g., Examples 12 to 15.

### Example 5

### Preparation of Cells Having the Ability to Differentiate into Other Cell Types by Incubation in Medium Containing Dexamethasone and/or One or More Other Modulators of the RhoA and/or ROCK Pathway, and Treatment with Protease: Method 1

This example describes how to produce the progenitor cells of the invention by incubating differentiated cells in medium containing dexamethasone and/or one or more other modulators of RhoA and/or ROCK pathway, and treating the cells with or without protease e.g., trypsin. The cells produced by this method are then tested for their ability to differentiate into adipocytes, as determined by the accumulation of fat as before.

Production of Cells Capable of Differentiating into Other Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium Dulbecco's Modified Eagle Medium High Glucose (DMEM-HG; e.g., Lonza Cat #12-604) supplemented with 10% FBS (fetal bovine serum), and incubated at 37° C. in a humidified atmosphere of 5% CO₂ in air until adherent. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area. Once all cells are attached, the medium is replaced with growth medium Dulbecco's Modified Eagle Medium Low Glucose (DMEM-LG) containing 0-3 mM glucose supplemented with about 0.01 μM to about 100 μM of fresh dexamethasone (purchased from Sigma, St. Louis, Mo.) for about 5 min to 24 hours. Alternatively, the medium is supplemented with effective amount(s) of dexamethasone and/or one or more other modulators of the RhoA and/or ROCK pathway e.g., Growth hormone (GH) (e.g., Novo Nordisk Pharma (Singapore)) and/or TNF-α and/or fibronectin (e.g., Sigma-Aldrich) and/or lysophosphatidic acid (LPA) (e.g., Toronto Research Chemicals Inc.), and/or Y-27632 (e.g., Alexis Biochemicals) and the cells incubated for about 5 min to 24 hours. Control cells are incubated with the same medium as test cells without dexamethasone or other modulators of the RhoA and/or ROCK pathway.

At the conclusion of the incubation period in media containing dexamethasone and/or one or more other modulators of the RhoA and/or ROCK pathway, cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (with 10% FBS) and maintained in this medium until required for re-differentiation. Alternatively test cells are not detached, and are used directly in the differentiation assay as described in Example 1. Differentiation into Adipocytes and assessment of adipogenesis is carried out as before.

For example, differentiation of the cell product into different cell types is achieved by reseeding the cells into differentiation media for adipocytes as in Example 1, or as described herein e.g., Examples 12 to 15.

### Example 6

### Preparation of Cells Having the Ability to Differentiate into Other Cell Types by Incubation in Medium Containing Dexamethasone and/or One or More Other Modulators of RhoA and/or ROCK Pathway, and Treatment with Protease, with Additional Incubation in Low-Serum: Method 2

This example describes how to produce the progenitor cells of the invention by incubating differentiated cells in medium containing dexamethasone and/or one or more other modulators of RhoA and/or ROCK pathway, and treating the cells with protease, and incubating cells in medium having low-serum concentration. This method may provide an increase in the proportion of cells achieving optimum plasticity compared to the treatment with dexamethasone and/or other modulator of RhoA and/or ROCK pathway alone.

Production of Cells Capable of Differentiating into Other Cell Types

Fresh adult human dermal fibroblasts are plated in DMEM-HG supplemented with 10% FBS, incubated and attached as described in Example 5 above. Once all cells are attached, the medium is replaced with medium 199 (M199) supplemented with 0-1% FBS (low-serum) for different periods of time, from 1 to 11 days.

The medium is then replaced with DMEM-LG containing 0-3 mM glucose supplemented about 0.01 μM to about 100 μM of fresh dexamethasone and cells are incubated from about 5 minutes to about 24 hours or 5 minutes to about 48 hours as described in Example 5. Alternatively, the medium is supplemented with effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to about 24 hours as described in Example 5. Control cells are incubated with the same medium as test cells without dexamethasone or other modulators of the RhoA and/or ROCK pathway.

At the conclusion of the incubation period in low serum media, test cells were detached by the addition of 20 μl of detachment solution as in Example 1 and incubated at 37° C. until cells lifted from the plates. Test cells were recovered from culture, then diluted to 200 μl with serum-free DMEM-HG (0% FBS) and maintained in serum-free medium until required for re-differentiation. Control cells are not detached, and are used directly in the differentiation assay as described in Example 1. Differentiation into Adipocytes and assessment of adipogenesis is carried out as described in Example 1. The person skilled in the art would appreciate that differentiation of test cells into adipocytes may continue albeit at below optimum even after the 11-day period incubation at low serum.

For example, differentiation of the cell product into other cell types is achieved by reseeding the cells described herein into differentiation media for adipocytes as described in Example 1, or as described herein e.g., Examples 12 to 15.

### Example 7

### Preparation of Cells Having the Ability to Differentiate into Other Cell Types by Incubation in Medium Containing Dexamethasone and/or One or More Other Modulators of RhoA and/or ROCK Pathway and Treatment with Protease and Additional Incubation at High Cell Density Conditions: Method 3

This example describes how to produce the progenitor cells of the invention by incubating differentiated cells e.g., fibroblasts in a medium containing dexamethasone and/or one or more other modulators of RhoA and/or ROCK pathway and treating the cells with protease, and incubating cells at high density conditions. This method may provide an increase in the proportion of cells achieving optimum plasticity compared to the treatment with dexamethasone and/or one or more other modulators of RhoA and/or ROCK pathway with or without protease treatment pathways alone.

In this set of experiments for producing cells having the ability to differentiate into different cell types, fresh human dermal fibroblasts derived from adult skin or from foreskin fibroblasts are cultured and detached by incubation with trypsin essentially as described in example 5. Test cells are then recovered from culture immediately after trypsinization and are diluted to about 100,000 cells in 100 μl in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and within about 4 to 6 hours after trypsinization, the recovered cells are seeded at concentrations of about 100,000 cells per well/plate or at about 3703.7 cells per mm² surface area of the well/plate before attachment of the cells to the plate/well directly in 400 μl high density plating medium (e.g., Medium 199 containing 170 nM insulin, 0.5 mM 3-isobutyl-1-methylxanthine, 0.2 mM indomethacin, 1 μM dexamethasone, and 15% rabbit serum) for a time and under conditions sufficient for an optimum number of progenitor cells to be produced e.g., for up to about 24 hours or until adherence is achieved i.e., a shorter time than required for cells to become adherent and/or as determined by analysis of cell marker expression and/or by the ability of aliquots of cells to subsequently undergo differentiation.

As a negative control for the production of progenitor cells, trypsinized cells are seeded at a reduced density i.e., about 740.1 cells per mm² surface area, in high density plating medium (e.g., Medium 199 containing 170 nM insulin, 0.5 mM 3-isobutyl-1-methylxanthine, 0.2 mM indomethacin, 1 μM dexamethasone, and 15% rabbit serum) and incubated as for samples seeded at high density e.g., for up to about 24 hours or until adherence is achieved i.e., a shorter time than required for cells to become adherent and/or as determined by analysis of cell marker expression and/or by the ability of aliquots of cells to subsequently undergo differentiation.

Differentiation into Adipocytes

For differentiation into adipocytes, cells are incubated in adipogenic medium (Medium 199 containing 170 nM insulin, 0.5 mM 3-isobutyl-1-methylxanthine, 0.2 mM indomethacin, 1 μM dexamethasone, and 15% rabbit serum) at high density and allowed to expand for about 10-21 days.

As a negative control for differentiation, trypsinized cells are seeded at high density in high density plating medium (e.g., DMEM-HG supplemented with 10% FBS) and incubated as for test samples seeded at high density e.g., for up to about 24 hours or until adherent and/or as determined by analysis of cell marker expression and/or by the ability of aliquots of cells to subsequently undergo differentiation. The high density plating medium is then replaced with 200 to 400 μl DMEM-HG (10% FCS) medium and cells are allowed to expand for about 10-21 days.

As positive control for differentiation, rat bone marrow stromal/stem cells (rBMSCs) are expanded in DMEM medium containing L-Glutamine and 10% FBS, and allowed to attach and reach sub-confluence or confluence. These cells are then detached by incubation with trypsin as described above, and seeded at concentration of about 50,000 cells per well/plate or at about 1851.9 cells per mm² surface area of the well/plate in 400 μl DMEM-HG containing 10% FBS for up to about 24 hours or until adherent. The medium is replaced from adherent culture with 200 to 400 ml adipogenic medium (Medium 199 containing 170 nM insulin, 0.5 mM 3-isobutyl-1-methylxanthine, 0.2 mM indomethacin, 1 μM dexamethasone, and 15% rabbit serum) and cells are allowed to expand for about 10-21 days.

Medium is replaced every 3 days for both test cells and negative and positive control cells.

### Assessment of Adipogenesis

After incubation for 12-21 days in adipogenic medium, differentiation potential of test cells compared to control cells at each day of incubation at high density post incubation optionally with low-serum and trypsinization is measured by an assessment of adipogenisis as described in example 1.

### Example 8

### Preparation of Cells Having the Ability to Differentiate into Other Cell Types by Incubation in a Medium Containing a Modulator of Src Pathway and Dexamethasone and/or One or More Other Modulators of RhoA and/or ROCK Pathway: Method 1

This example describes how to produce the progenitor cells of the invention by incubating differentiated cells e.g., human fibroblasts in a medium containing one or more modulators of SRC pathway and dexamethasone and/or one or more other modulators of RhoA and/or ROCK pathway, and with or without treatment of the cells with a protease e.g., trypsin. The cells produced by this method are then tested for their ability to differentiate into adipocytes, as determined by the accumulation of fat. This method may provide an increase in the proportion of cells achieving optimum plasticity compared to the treatment of the differentiated cells with one or more modulators of SRC pathway alone. This method may also provide an increase in the proportion of cells achieving optimum plasticity compared to the treatment of the differentiated cells with dexamethasone or other modulator of RhoA or ROCK pathway alone.

Production of Cells Capable of Differentiating into Other Cell Types

Fresh human dermal fibroblasts derived from adult skin or from foreskin are plated in cell culture flasks, or plates, in growth medium DMEM-HG supplemented with 10% FBS and incubated until adherent, as described before. Human dermal fibroblasts are plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area.

Once all cells are attached, the medium is replaced with DMEM-LG containing 0-3 mM glucose supplemented with about 0.01 μM to about 100 μM, preferably 1 μM to about 10 μM recombinant human FGF-1 (Sigma-Aldrich or R&D Systems) e.g., to activate the SRC pathway and with about 0.01 μM to about 100 μM of fresh dexamethasone (Sigma) for about 5 min to 48 hours. Alternatively, the medium is supplemented with about 0.01 μM to about 100 μM, preferably 1 μM to about 10 μM recombinant human FGF-1 and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 48 hours. Control cells are incubated with the same medium as test cells without FGF-1 and without dexamethasone or other modulators of the RhoA and/or ROCK pathway.

Alternatively, once all cells are attached, the medium is replaced with DMEM-LG containing 0-3 mM glucose supplemented with about 0.01 μM to about 100 μM recombinant human HGF (R&D systems) e.g., to activate the SRC pathway and with about 0.01 μM to about 100 μM of fresh dexamethasone (Sigma) for about 5 min to 24 hours. Alternatively, the medium is supplemented with about 0.01 μM to about 100 μM recombinant human HGF and effective amount(s) of dexamethasone and/or one or more other modulators of the RhoA and/or ROCK pathway e.g., GH and/or TNF-α and/or fibronectin and/or LPA and/or Y-27632 and the cells are incubated for about min to 24 hours. Control cells are incubated with the same medium as test cells without HGF and without dexamethasone or other modulators of the RhoA and/or ROCK pathway.

Alternatively, once all cells are attached, the medium is replaced with DMEM-LG containing 0-3 mM glucose supplemented with about 0.01 μM to about 100 μM recombinant NGFβ (Boehringer Mannheim) e.g., to activate the SRC pathway and about 0.01 μM to about 100 μM of fresh dexamethasone (Sigma) for about 5 min to 24 hours. Alternatively, the medium is supplemented with about 0.01 μM to about 100 μM recombinant NGFβ and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without NGF and without dexamethasone or other modulators of the RhoA and/or ROCK pathway.

Alternatively, once all cells are attached, the medium is replaced with DMEM-LG containing 0-3 mM glucose supplemented with about 0.01 μM to about 100 μM rat recombinant IL-10 (Sigma) e.g., to activate the SRC pathway and about 0.01 μM to about 100 μM of fresh dexamethasone (Sigma) for about 5 min to 24 hours. Alternatively, the medium is supplemented with about 0.01 μM to about 100 μM rat recombinant IL-10 and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without IL-10 and without dexamethasone or other modulators of the RhoA and/or ROCK pathway.

Alternatively, once all cells are attached, the medium is replaced with DMEM-LG containing 0-3 mM glucose supplemented with about 0.01 μM to about 100 μM NT3 (Regeneron Pharmaceuticals) e.g., to activate the SRC pathway and about 0.01 μM to about 100 μM of fresh dexamethasone (Sigma) for about 5 min to 24 hours. Alternatively, the medium is supplemented with about 0.01 μM to about 100 μM NT3 and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without NT3 and without dexamethasone or other modulators of the RhoA and/or ROCK pathway.

Alternatively, once all cells are attached, the medium is replaced with DMEM-LG containing 0-3 mM glucose supplemented with about 0.01 μM to about 100 μM recombinant SEMA-3A (R&D systems) e.g., to activate the SRC pathway and about 0.01 μM to about 100 μM of fresh dexamethasone (Sigma) for about 5 min to 24 hours. Alternatively, the medium is supplemented with about 0.01 μM to about 100 μM recombinant SEMA-3A and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without SEMA-3A and without dexamethasone or other modulators of the RhoA and/or ROCK pathway.

At the conclusion of the incubation period in media containing FGF-1, HGF, NGFβ, IL-1β, NT3, or SEMA-3A and dexamethasone and/or one or more other modulators of the RhoA and/or ROCK pathway cells are washed in PBS and the medium is replaced with growth medium Dulbecco's Modified Eagle Medium High Glucose (DMEM-HG; e.g., Lonza Cat #12-604) supplemented with 10% FBS (fetal bovine serum), test cells are detached by the addition of 20 μl of detachment solution containing 0.12% trypsin, 0.02% EDTA and 0.04% glucose (SAFC Biosciences, Cat #59430C) and incubated at 37° C. until cells lifted from the plates. Test cells are recovered from culture, then diluted to 200 μl with DMEM-HG (e.g., Lonza, Cat #12-604) (with 10% FBS) and maintained in this medium until required for re-differentiation. Alternatively test cells are not detached, and are used directly in the differentiation assay as described in Example 1. Differentiation into Adipocytes and assessment of adipogenesis is carried out as before.

For example, differentiation of the cell product into different cell types is achieved by reseeding the cells into differentiation media for adipocytes as in Example 1, or as described herein e.g., Examples 12 to 15.

### Example 9

### Preparation of Cells Having the Ability to Differentiate into Other Cell Types by Incubation in a Medium Containing a Modulator of Src Pathway and Dexamethasone and/or One or More Other Modulators of RhoA and/or ROCK Pathway: Method 2

This is a further example describing how to produce the progenitor cells of the invention by incubating differentiated cells in a medium containing one or more modulators of SRC pathway and dexamethasone and/or one or more other modulators of RhoA and/or ROCK pathway, and with or without treatment of the cells with a protease e.g., trypsin.

Production of Cells Capable of Differentiating into Different Cell Types

Human myometrial microvascular endothelial cells (HUMEC) are obtained from Technoclone GmbH (Vienna, Austria) and are cultured at 37 C in endothelial growth medium and incubated until adherent according to the specifications supplied by Technoclone GmbH. Alternatively, non-transformed rat embryo fibroblasts (Rat-1) are prepared and maintained as previously described (Peterson, et al., J. Biol. Chem. 271:31562-31571 (1996)). HUMEC cells are plated in two sets, one set of cells are used as control cells, and the second set of cells are used for testing the capability of cells produced by the method to differentiate into adipocytes. Rat-1 cells are also plated in two sets, one set of cells are used as control cells, and the second set of cells are used for testing the capability of cells produced by the method to differentiate into adipocytes. Control cells are plated directly onto 96 well plates at about 20,000 cells per well or about 740.74 cells per mm² surface area. Test cells are plated onto larger plates but at the same concentration of cells per well or cells per mm² surface area.

Adherent HUMEC cultures or adherent Rat-1 cultures are then incubated in DMEM-LG culture medium containing 0-3 mM glucose supplemented with about 0.01 μM to about 100 μM, preferably 1 μM to about 10 μM recombinant human FGF-1 (Sigma-Aldrich or R&D Systems) e.g., to activate the SRC pathway and with about 0.01 μM to about 100 μM of fresh dexamethasone (Sigma) for about 5 min to 48 hours. Alternatively, the medium is supplemented with about 0.01 μM to about 100 μM, preferably 1 μM to about 10 μM recombinant human FGF-1 and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 48 hours. Control cells are incubated with the same medium as test cells without FGF-1 and without dexamethasone or other modulators of the RhoA and/or ROCK pathway.

Alternatively, the medium is replaced with DMEM-LG containing 0-3 mM glucose supplemented with about 0.01 μM to about 100 μM recombinant human HGF (R&D systems) e.g., to activate the SRC pathway and with about 0.01 μM to about 100 μM of fresh dexamethasone (Sigma) for about 5 min to 24 hours. Alternatively, the medium is supplemented with about 0.01 μM to about 100 μM recombinant human HGF and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without HGF and without dexamethasone or other modulators of the RhoA and/or ROCK pathway.

Alternatively, the medium is replaced with DMEM-LG containing 0-3 mM glucose supplemented with about 0.01 μM to about 100 μM recombinant NGFβ (Boehringer Mannheim) e.g., to activate the SRC pathway and about 0.01 μM to about 100 μM of fresh dexamethasone (Sigma) for about min to 24 hours. Alternatively, the medium is supplemented with about 0.01 μM to about 100 μM recombinant NGFβ and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without NGFβ and without dexamethasone or other modulators of the RhoA and/or ROCK pathway.

Alternatively, the medium is replaced with DMEM-LG containing 0-3 mM glucose supplemented with about 0.01 μM to about 100 μM rat recombinant IL-1β (Sigma) e.g., to activate the SRC pathway and about 0.01 μM to about 100 μM of fresh dexamethasone (Sigma) for about 5 min to hours. Alternatively, the medium is supplemented with about 0.01 μM to about 100 μM rat recombinant IL-1β and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without IL-1β and without dexamethasone or other modulators of the RhoA and/or ROCK pathway.

Alternatively, the medium is replaced with DMEM-LG containing 0-3 mM glucose supplemented with about 0.01 μM to about 100 μM NT3 (Regeneron Pharmaceuticals) e.g., to activate the SRC pathway and about 0.01 μM to about 100 μM of fresh dexamethasone (Sigma) for about 5 min to hours. Alternatively, the medium is supplemented with about 0.01 μM to about 100 μM NT3 and effective amount(s) of dexamethasone and/or one or more other modulators of the RhoA and/or ROCK pathway e.g., GH and/or TNF-α and/or fibronectin and/or LPA and/or Y-27632 and the cells are incubated for about 5 min to 24 hours. Control cells are incubated with the same medium as test cells without NT3 and without dexamethasone or other modulators of the RhoA and/or ROCK pathway.

Alternatively, the medium is replaced with DMEM-LG containing 0-3 mM glucose supplemented with about 0.01 μM to about 100 μM recombinant SEMA-3A (R&D systems) e.g., to activate the SRC pathway and about 0.01 μM to about 100 μM of fresh dexamethasone (Sigma) for about 5 min to 24 hours. Alternatively, the medium is supplemented with about 0.01 μM to about 100 μM recombinant SEMA-3A and effective amount(s) of dexamethasone and/or one or more other modulators of the RhoA and/or ROCK pathway e.g., GH and/or TNF-α, and/or fibronectin and/or LPA and/or Y-27632 and the cells are incubated for about 5 mM to 24 hours. Control cells are incubated with the same medium as test cells without SEMA-3A and without dexamethasone or other modulators of the RhoA and/or ROCK pathway.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Differentiation into Other Cell Types

Re-differentiation of the treated adrenal cells into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Example 1 and Examples 12 to 15.

### Example 10

### Preparation of Cells Having the Ability to Differentiate into Other Cell Types by Induction of the Akt/(PKB) Pathway

This example describes how to produce the progenitor cells of the invention by incubating differentiated cells in medium containing a modulator of SRC pathway or in a medium containing dexamethasone and/or one or more other modulators of RhoA and/or ROCK pathway, and with or without treatment of the cells with a protease e.g., trypsin, and further incubating the cells in the presence of one or more agonists of the Akt/(PKB) pathway. This method may provide an increase in the proportion of cells achieving optimum plasticity compared to the treatment of the differentiated cells with one or more modulators of SRC pathway alone. This method may also provide an increase in the proportion of cells achieving optimum plasticity compared to the treatment of the differentiated cells with dexamethasone or other modulator of RhoA or ROCK pathway alone.

Without being bound by theory or mode of action, the inventor reasons that by enhancing induction of the Akt/(PKB) pathway using an agonist compound, differentiated primary cells and cell lines that would normally enter a quiescent state or undergo apoptosis following modulation of SRC and/or modulation of RhoA and/or ROCK and/or incubation with dexamethasone, may also be used to produce cells capable of differentiating into different cell types.

In one example to show that Akt/(PKB) pathway induction may confer or enhance plasticity of fibroblasts, primary human foreskin fibroblasts are incubated in the presence of PDGF-BB or TGF-β or sodium pyruvate or carbachol or IGF-1 to induce the Akt/(PKB) pathway.

Production of Cells Capable of Differentiating into Different Cell Types

Fresh human dermal fibroblasts that are derived from adult skin or from foreskin are purchased from PromoCell® (Banksia Scientific Company, QLD). Human dermal fibroblasts are plated in cell culture flasks, or plates, in growth medium (DMEM-HG; e.g., Lonza) supplemented with 10% FBS (fetal bovine serum), and incubated at 37° C. in a humidified atmosphere of 5% CO₂ in air until adherent. Once all cells are attached, the medium is replaced with DMEM-HG (e.g., Lonza) supplemented with 0-1% FBS or BSA (low-protein) for 24 hours to precondition the cells for PDGF-BB. After 24 hours, the medium is replaced with low-serum or serum-free DMEM containing 10 to 100 ng/ml of human recombinant PDGF-BB (Invitrogen) for 5 to 15 min or alternatively with 1 to 10 ng/ml of TGF-β (R&D systems) for at least 60 min, or alternatively with to 200 mg/L of cell culture grade sodium pyruvate (e.g., Lonza), and preferably, at 110 mg/L for at least 1 h, or alternatively with 200-1000 μM Carbachol (Calbiochem, San Diego, Calif.) or at least 50 ng/ml purified NGF (2.5S) (Alomone Labs Ltd) for 5 to 10 min, or alternatively with at least 250 ng/ml of IGF-1 (Sigma) for at least about 20 min, to activate the Akt/(PKB) pathway.

The medium is then replaced with DMEM-LG containing 0-3 mM glucose supplemented with about 0.01 μM to about 100 μM, preferably 1 μM to about 10 μM FGF-1 from about 5 minutes to about 48 hours and incubated as described in Example 1. Control cells are incubated with the same medium as test cells but without FGF-1. Alternatively, the medium is replaced with DMEM-HG supplemented with 0.01 μM to about 100 μM HGF for about 5 min to about 48 hours and incubated as described in Example 1. Control cells are incubated with the same medium as test cells without HGF. Alternatively, the medium is replaced with DMEM-LG containing 0-3 mM glucose supplemented with about 0.01 μM to about 100 μM recombinant NGFβ for about 5 min to about 24 hours and incubated as described in Example 1. Control cells are incubated with the same medium as test cells without NGFβ. Alternatively, the medium is replaced with DMEM-LG containing 0-3 mM glucose supplemented with about 0.01 μM to about 100 μM rat recombinant IL-1β for about 5 min to about 24 hours, and incubated as described in Example 1. Control cells are incubated with the same medium as test cells without IL-1β. Alternatively, the medium is replaced with DMEM-HG containing supplemented with about 0.01 μM to about 100 μM NT3 for about 5 min to about 24 hours, and incubated as described in Example 1. Control cells are incubated with the same medium as test cells without NT3. Alternatively, the medium is replaced with DMEM-HG supplemented with about 0.01 μM to about 100 μM recombinant SEMA-3A for about 5 min to about 24 hours, and incubated as described in Example 1. Control cells are incubated with the same medium as test cells without SEMA-3. Alternatively, the medium is replaced with DMEM-HG supplemented with about 0.01 μM to about 100 μM of fresh dexamethasone for about 5 min to about 24 hours, and incubated as described in Example 5. Control cells are incubated with the same medium as test cells without dexamethasone. Alternatively, the medium is supplemented with effective amount(s) of dexamethasone and/or one or more other modulators of the RhoA and/or ROCK pathway e.g., GH and/or TNF-α and/or fibronectin and/or LPA and/or Y-27632 and the cells incubated for about 5 min to 24 hours. Control cells are incubated with the same medium as test cells without dexamethasone or other modulators of the RhoA and/or ROCK pathway as described in Example 5.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (e.g., Lonza) (10% FBS) and maintained in this medium until required for re-differentiation.

Differentiation into Other Cell Types

Re-differentiation of the treated fibroblasts into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Example 1 and Examples 12 to 15.

### Example 11

### Preparation of Cells Having the Ability to Differentiate into Other Cell Types by Induction of the NF-κB Pathway

This example describes how to produce the progenitor cells of the invention by incubating differentiated cells in medium containing a modulator of SRC pathway or in a medium containing dexamethasone and/or one or more other modulators of RhoA and/or ROCK pathway, and with or without treatment of the cells with a protease e.g., trypsin, and further incubating the cells in the presence of one or more agonists of the NF-κB pathway. This method may provide an increase in the proportion of cells achieving optimum plasticity compared to the treatment of the differentiated cells with one or more modulators of SRC pathway alone. This method may also provide an increase in the proportion of cells achieving optimum plasticity compared to the treatment of the differentiated cells with dexamethasone or other modulator of RhoA or ROCK pathway alone.

Without being bound by theory or mode of action, the inventor reasons that by enhancing induction of the NF-κB pathway using an agonist compound, differentiated primary cells and cell lines that would normally enter a quiescent state or undergo apoptosis following modulation of SRC and/or modulation of RhoA and/or ROCK and/or incubation with dexamethasone, may also be used to produce cells capable of differentiating into different cell types.

In one example to show that induction of NF-κB pathway may confer or enhance plasticity of fibroblasts, primary human foreskin fibroblasts are incubated in the presence of TNF-α or IL-1α or LPS to induce the NF-κB pathway.

Production of Cells Capable of Differentiating into Different Cell Types

Fresh human dermal fibroblasts derived from adult skin or from foreskin are purchased from PromoCell® (Banksia Scientific Company, QLD). Human dermal fibroblasts are plated in cell culture flasks, or plates, in growth medium (DMEM-HG) supplemented with 10% FBS (fetal bovine serum), and incubated at 37° C. in a humidified atmosphere of 5% CO₂ in air until adherent. Once all cells are attached, the medium is replaced with serum-free DMEM or low-serum DMEM-HG containing at least 20 ng/ml of TNF-α (Roche) for at least 60 min to activate the NF-KB pathway. Alternatively once all cells are attached, the medium is replaced with DMEM supplemented with 0.25% FBS of BSA for 50 hours to precondition the cells to interleukin-1α. After 50 hours, the cells are treated with recombinant human IL-1α at a concentration of least 0.27 ng/ml to activate the NF-κB pathway. Alternatively, once all cells are attached the medium is replaced with DMEM supplemented with 10-100 ng/ml of Lipopolysaccharide (LPS; Sigma) for at least 45 min to activate the NF-κB pathway.

The medium is then replaced with DMEM-LG containing 0-3 mM glucose supplemented with about 0.01 μM to about 100 μM, preferably 1 μM to about 10 μM FGF-1 from about 5 minutes to about 48 hours and incubated as described in Example 1. Control cells are incubated with the same medium as test cells but without FGF-1. Alternatively, the medium is replaced with DMEM-HG supplemented with 0.01 μM to about 100 μM HGF for about 5 min to about 48 hours and incubated as described in Example 1. Control cells are incubated with the same medium as test cells without HGF. Alternatively, the medium is replaced with DMEM-LG containing 0-3 mM glucose supplemented with about 0.01 μM to about 100 μM NGFβ for about 5 min to about hours and incubated as described in Example 1. Control cells are incubated with the same medium as test cells without NGFβ. Alternatively, the medium is replaced with DMEM-LG containing 0-3 mM glucose supplemented with about 0.01 μM to about 100 μM rat recombinant IL-1β for about 5 min to about 24 hours, and incubated as described in Example 1. Control cells are incubated with the same medium as test cells without IL-1β. Alternatively, the medium is replaced with DMEM-HG containing supplemented with about 0.01 μM to about 100 μM NT3 for about 5 min to about 24 hours, and incubated as described in Example 1. Control cells are incubated with the same medium as test cells without NT3. Alternatively, the medium is replaced with DMEM-HG supplemented with about 0.01 μM to about 100 μM SEMA-3A for about 5 min to about 24 hours, and incubated as described in Example 1. Control cells are incubated with the same medium as test cells without SEMA-3. Alternatively, the medium is replaced with DMEM-HG supplemented with about 0.01 μM to about 100 μM of fresh dexamethasone for about 5 min to about 24 hours, and incubated as described in Example 5. Control cells are incubated with the same medium as test cells without dexamethasone. Alternatively, the medium is supplemented with effective amount(s) of dexamethasone and/or one or more other modulators of the RhoA and/or ROCK pathway e.g., GH and/or TNF-α and/or fibronectin and/or LPA and/or Y-27632 and the cells incubated for about 5 min to 24 hours. Control cells are incubated with the same medium as test cells without dexamethasone or other modulators of the RhoA and/or ROCK pathway as described in Example 5.

Preferably, treated adherent cells are detached from plates by the addition of 20 μl of detachment solution containing 0.12% Trypsin, 0.02% EDTA and 0.04% Glucose (SAFC Biosciences, Cat #59430C) and are incubated at 37° C. until cells lifted from the plates. Treated cells are recovered from culture, then diluted to 200 μl with DMEM-HG (10% FBS) and maintained in this medium until required for re-differentiation.

Differentiation into Other Cell Types

Re-differentiation of the treated adrenal cells into other cell types is achieved by reseeding the treated cells described above into differentiation media, preferably after trypsinization and before reattachment. Methods suitable for differentiation of these cells into adipocytes, cells of osteogenic lineage, chondrogenic lineage, haematopoietic cells or insulin secreting cells are known in the art and described herein e.g., Examples 1 and Examples 12 to 15.

### Example 12

### Differentiation of Cells into Cells of Osteogenic Lineage

This example describes methods for producing cells of osteogenic lineage from the cell product of any one of Examples 1 through 11 that is capable of being differentiated into a different cell type. This example also describes methods for testing that osteogenic cells are produced.

### Differentiation Conditions

Cells capable of producing other cell types are prepared as in any one of Examples 1 to 11.

To produce cells of osteogenic lineage from such cells, the cells are incubated in complete osteogenic media (+DEX: DMEM-low glucose containing 10% FBS, 20 μg/ml ascorbic acid phosphate-magnesium salt, 1.5 mg/ml beta glycerophosphate and 40 ng/ml dexamethasone) for differentiation to the osteogenic lineage, or in incomplete osteogenic media (−DEX: DMEM-low glucose containing 10% FBS, 20 ug/ml ascorbic acid phosphate-magnesium salt, 1.5 mg/ml beta glycerophosphate), as a control. The cells are then plated in their respective media onto 96-well plates at about 20,000 cells per well or about 740.74 cells per mm² surface area of the well for alkaline phosphatase assays (ALP) or at 50,000 cells per well or about 1851.85 cells per mm² surface area of the well for mineral deposition assays as described below. Alternatively, the cells are plated in their respective differentiation media onto 96-well plates at about 100,000 cells per well or at about 3703.7 cells per mm² surface area of the well where optional high density plating step is employed e.g., as in Examples 4 and 7. Complete or incomplete osteogenic media is replaced every 3 days.

### Assessment of Osteogenesis Using an Alkaline Phosphatase (ALP) Assay

After incubation for 12-21 days in either complete or incomplete osteogenic media as described above, alkaline phosphatase is assessed. The media is removed from cells; cells are washed in phosphate buffered saline and lysed with 40 μl of Passive Lysis Buffer (Promega). The lysate is sonicated. After sonication, the lysate is split into two equal samples of 20 μL each. One sample is placed into a separate 48 well plate, Add 180 uL of Hoescht 33258 in buffer (5 μg/mL in 2M NaCl or 20×SSC) (i.e 1:9 ratio of PLB to Hoescht) is added, and the sample is read at Excitation nm/Emission 460 on Molecular Probes fluorescent scanner. p-Nitrophenyl phosphate (pNPP) 75 μL is added to the remaining sample and incubated for 30 minutes at 37° C. One hundred (100) μl of 2M NaOH is subsequently added which will turn into yellow p-Nitrophenylene anion—pNP. An aliquot of 100 μl is transferred to a 96 well plate for plate reading. The absorbance of pNP (yellow) is read on an optical plate reader at 405 nm. A comparison of +Dex to −Dex controls of Absorbance/ng DNA using a PNPP standard curve is made.

### Assessment of Mineral Deposition

After incubation for 21 days in either complete or incomplete osteogenic media as described above, mineral deposition is assessed. To test for mineral deposition, cells are stained with Von Kossa. A comparison of staining intensity is performed on +Dex differentiated cells to −Dex treated controls.

### Example 13

### Differentiation of Cells into Cells of Chondrogenic Lineage

This example describes methods for producing cells of chondrogenic lineage from the cell product of any one of Examples 1 through 11 that is capable of being differentiated into a different cell type. This example also described methods for testing that chondrogenic cells are produced.

### Differentiation Conditions

Cells capable of producing other cell types are prepared as in any one of Examples 1 to 11.

To produce cells of chondrogenic lineage from such cells, the cells incubated in chondrogenic media (DMEM-HG containing ITS+ supplement at a 1 fold concentration (final concentrations of 6.25 μg/ml bovine insulin; 6.25 μg/ml transferrin; 6.25 μg/ml selenous acid; 5.33 μg/ml linoleic acid; 1.25 mg/ml BSA) 50 μg/ml ascorbic acid-2-phosphate, 40 μg/ml L-proline, 100 mg/ml pyruvate, 100 nM dexamethasone, 10 ng/ml TGF-β, and 500 ng/ml BMP-2) for differentiation to the chondrogenic lineage; or in DMEM-HG containing 1.25 mg/ml BSA, as a control. The cells are then plated in their respective media onto 96-well plates at about 20,000-50,000 cells per well or at about 740.74-1851.85 cells per mm² plating surface area of the well. Alternatively, the cells are plated in their respective differentiation media onto 96-well plates at about 100,000 cells per well or at about 3703.7 cells per mm² plating surface area of the well where optional high density plating step is employed e.g., as in Examples 4 and 7. Chondrogenic media or control media is replaced every 3 days.

### Assessment of Chondrogenesis

After incubation for 12-21 days in either chondrogenic media or control media as described above, cells are assessed by observation for the appearance of chondrocyte morphology. Analysis of the accumulation of sulfated glycosaminoglycans (GAG) is carried out by measuring the amount of 1,9-dimethylmethylene blue-reactive material in extracts of cells treated with chondrogenic media and compared with extracts of control cells. The 1,9-dimethylmethylene blue assay is performed essentially as described in Sabiston et al, Analytical Biochemistry 149: 543-548 (1985).

### Example 14

### Differentiation of Cells into Haematopoietic Cells

This example describes methods for producing haematopoietic cells from the cell product of any one of Examples 1 through 11 that is capable of being differentiated into a different cell type. This example also described methods for testing that haematopoietic cells are produced.

### Differentiation Conditions

Cells capable of producing other cell types are prepared as in any one of Examples 1 to 11.

To produce haematopoietic cells from such cells, the cells are mixed with DMEM supplemented with Granulocyte macrophage colony-stimulating factor (GM-CSF; 50 ng/ml) and stem cell factor (SCF; 50 ng/ml), plated onto 35-mm tissue culture dishes and are incubated at 37° C. in a humidified atmosphere of 5% CO₂ in air for 2 days. The cells are harvested and analyzed for cells expressing the hematopoietic marker CD45 by flow cytometry.

To detect the presence of the cell surface CD45 antigen, cells are incubated for 30 min. at 37° C. with anti-CD45 antibodies (Becton Dickinson), washed in PBS and analysed by flow cytometry. Flow cytometric analysis is performed using a FACSCalibur flow cytometer and the CellQuest software program (Becton Dickinson Immunocytometry Systems, San Jose, Calif.). Data analysis is performed using CellQuest and the Modfit LT V2.0 software program (Verity Software House, Topsham, Me.).

### Example 15

### Differentiation of Cells into Insulin-Secreting Cells

This example describes methods for producing insulin-secreting cells from the cell product of any one of Examples 1 through 11 that is capable of being differentiated into a different cell type. This example also described methods for testing that insulin-secreting cells are produced.

### Differentiation Conditions

Cells capable of producing other cell types are prepared as in any one of Examples 1 to 11.

To produce insulin-secreting cells from such cells, the cells are plated into serum free medium to enrich for nestin-positive cells (see Lumelsky et al., Science, 292:1389, 2001). The nestin-positive cells are then sub-subcultured and expanded for 6 to 7 days in serum-free N2 media supplemented with 1 μg/ml laminin, 10 ng/ml bFGF, 500 ng/ml N-terminal fragment of murine or human SHH (sonic hedge hog) 100 ng/ml FGF8 and B27 media supplement, as described in Lee et al. Nature Biotechnology, 18: 675 (2000) and Lumelsky (supra), which are herein incorporated by reference. After the nestin-positive cells are expanded, the growth factors (FGF, SHH) are removed from the media and nicotinamide is added to the media at a final concentration of 10 mM, to promote the cessation of cell proliferation and induce the differentiation of insulin-secreting cells. After approximately 6 days of growth factor starvation, aggregates of insulin-secreting cells are formed (islet-like cell clusters), which are autologous to the individual from whom they are derived.

### Example 16

### Multipotency of Cells Produced in Accordance with the Invention

### Retinoic Acid-Induced Differentiation of the Cells

Cells are tested for an ability to regenerate their telomeres, as determined by expression of telomerase. The expression of relatively high levels of telomerase in a cell culture is indicative of a stem cell-like phenotype. Furthermore, retinoic acid (RA)-induced differentiated cells down-regulate the expression of telomerase and express genes indicative of differentiating cells of various lineages. For example, Schuldiner et al., PNAS 97:11307 (2000) demonstrated the increased expression of tissue specific lineage markers, e.g., brain-specific neurofilament (ectodermal), heart-specific cardiac actin (mesodermal) and liver-specific α1-antitrypsin (endodermal), in cultures of human embryonic stem cells treated with RA.

Cells prepared as described in any one of Examples 1 to 11 are cultured in the presence of approximately 1-2 μM RA (Sigma, St. Louis) for 5 to 10 days, optionally in low-serum medium and/or the presence of one or more agonists of the Akt/(PKB) pathway and/or NF-κB pathway to maintain their plasticity.

### RNA Extraction and RT-PCR

To monitor the differential expression of various genes in the cells, reverse transcription-polymerase chain reaction (RT-PCR) is performed. RNA is extracted from untreated cells and cells treated with RA, e.g., using Perfect RNA™ Eukaryotic Kit (Eppendorf A G, Hamburg, D E), essentially according to the manufacturer's instructions. The extracted RNA is dissolved in RNase-free water e.g., provided in the Perfect RNA™ Eukaryotic Kit.

RT-PCR is performed using the QIAGEN® OneStep RT-PCR Kit (Qiagen Inc., Valencia, Calif.) according to the manufacturer's instructions. PCR amplification is preformed using the following protocol: 94° C. for 1 min., 55° C. for 1 min., 72° C. for 1 min., for 45 cycles.

The oligonucleotide primers set out below are used to detect the following mRNAs: human telomerase (“TRT”), neurofilament heavy chain (“NF”), alpha-antitrypsin (“αAT”) and cardiac actin (“cACT”). To control for the quality of the extracted RNA and to serve as an internal quantification marker, human glyceraldehyde 3-phosphate dehydrogenase (“GAPDH”) oligonucleotide primers are included in the RT-PCR reaction.

### RT-PCR Primer Sets:

GAPDH
5′-GGGGAGCCAAAAGGGTCATCATCT-3-′;
5′-GACGCCTGCTTCACCACCTTCTTG-3′
TRT
5′-CGGAGGTCATCGCCAGCATCATCA-3-′
5′-GTCCCGCCGAATCCCCGCAAACAG-3′
NF
5′-TGAACACAGACGCTATGCGCTCAG-3′
5′-CACCTTTATGTGAGTGGACACAGAG-3′
αAT
5′-AGACCCTTTGAAGTCAAGGACACCG-3′
5′-CCATTGCTGAAGACCTTAGTGATGC-3′
cACT
5′-TCTATGAGGGCTAGCCTTTG-3′
5′-CCTGACTGGAAGGTAGATGG-3′

The RT-PCR products are electrophoresed on 2% (w/v) agarose gels stained with ethidium bromide. The intensities of the DNA product bands are quantified e.g., using PHORETIX™ TotalLab densitometry software package developed by Nonlinear USA (Durham, N.C.). To determine the approximate relative percent change in the expression of TRT, NF, αAT and cACT in each of the experimental groups relative to the untreated fibroblasts the following equation is applied (Eq. 1):

x=([(a′/b′)/(a/b)]−1)100% Eq. 1

wherein x is the relative percent change in expression of the gene of interest; b is the intensity of the GAPDH band in untreated fibroblasts; b′ is the intensity of the GAPDH band obtained from the experimental cells; a is the intensity of the gene-of-interest band obtained from the untreated fibroblasts; and a′ is the intensity of the gene-of-interest band obtained from the experimental cells.

Cells that are ectodermal-like, or mesodermal-like or endodermal-like can be differentiated into specialized tissues normally derived from each embryonic layer and subsequently used for treatment and/or therapy of disease.

### Example 17

### Therapy Using Differentiated Cells Produced in Accordance with the Invention

This example describes therapeutic applications of progenitor cells produced from differentiated cells in accordance with the inventive method.

### Diabetes

To treat human patients suffering from diabetes, progenitor cells are produced from differentiated cells and then differentiated into insulin-secreting cells as described in Example 15. Preferably, the differentiated cells used as starting material in this process were derived from the same patient or a matched patient to minimize or eliminate the risk of graft rejection. The insulin-secreting cells are grafted subcutaneously into a subject suffering from diabetes, wherein the cells are either encapsulated in a polymer matrix or non-encapsulated and containing a suitable isotonic buffer, or surgically infused into the patient's pancreas. A therapeutic amount of insulin-secreting cells are implanted in the patient subcutaneously. The skilled practitioner may determine a therapeutic amount based upon the age, weight and general health of the patient and the amount of insulin secreted by said insulin-secreting cells in response to glucose administration. Blood glucose levels of the patient are monitored on a regular basis and the amount of implanted cells are adjusted accordingly.

### Osteoarthritis

To treat human patients suffering from degenerative osteoarthritis, progenitor cells are produced from differentiated cells and then differentiated into chondrocytes as described in Example 13. Preferably, the differentiated cells used as starting material in this process were derived from the same patient or a matched patient to minimize or eliminate the risk of graft rejection. Cells of chondrocyte lineage are grafted into the diseased joints of a patient by implantation with a needle, or by orthoscopic surgical methods, wherein the cells are either encapsulated in a polymer matrix or non-encapsulated and containing a suitable isotonic buffer. Again, a therapeutic amount of chondrocytes are implanted in the patient's degenerated joints. The skilled practitioner may determine a therapeutic amount based upon the age, weight and general health of the patient and the disease progression in the patient.

The claims defining the invention are as follows:

1. A method for producing a progenitor cell capable of being differentiated into a plurality of different cell types, said method comprising incubating differentiated cells in a medium comprising an amount of one or more modulators of RhoA and/or ROCK pathway for a time and under conditions sufficient to produce a progenitor cell that is capable of being differentiated into a plurality of different cell types.

2. The method of claim 1, wherein the one or more modulators of RhoA and/or ROCK pathway is dexamethasone.

3. The method according to claim 1, wherein the method comprises incubating differentiated cells in a medium comprising an amount of one or more modulators of RhoA and/or ROCK pathway selected from the group consisting of dexamethasone, growth hormone (GH), tumor necrosis factor-α (TNF-α), fibronectin, lysophosphatidic acid, serum, Y-27637 and combinations thereof.

4. The method according to claim 1 or claim 3, wherein the method comprises incubating differentiated cells in a medium comprising a plurality of modulators of RhoA and/or ROCK pathway.

5. The method according to claim 4, wherein one of the plurality of modulators of RhoA and/or ROCK pathway is dexamethasone.

6. The method according to any one of claims 1 to 5, wherein the one or more modulators of RhoA and/or ROCK pathway include one or more agonists or partial agonists or reverse antagonists of RhoA and/or ROCK pathway.

7. The method according to any one of claims 1 to 6, wherein the one or more modulators of RhoA and/or ROCK pathway are capable of inducing de-differentiation of the differentiated cells into the progenitor cells.

8. The method according to any one of claims 1 to 7, further comprising incubating the differentiated cells in a medium comprising an amount of one or more modulators of SRC pathway for a time and under conditions sufficient to produce a progenitor cell that is capable of being differentiated into a plurality of different cell types.

9. The method of claim 8, wherein the one or more modulators of SRC pathway are selected form the group consisting of transforming growth factor (TGF), transforming growth factor beta-1 (TGF-β1), nerve growth factor-βa (NGFβ), interleukin 1-β (IL-1β), Fibroblast growth factor-1 (FGF-1), fibroblast growth factor-2 (FGF-2), hepatocyte growth factor (HGF), neurotrophin 3 (NT3), semaphorin (SEMA), semaphorin-3A (SEMA-3A), platelet-derived growth factor (PDGF), platelet-derived growth factor B-chain (PDGF-B), 4-amino-5-(4-methylphenyl)-7-(t-butyl)pyrazolo (3,4-d) pyrimidine, (PP1), 4-Amino-5-(4-chlorophenyl)-7-(t-butyl)pyrazolo[3,4-d]pyrimidine (PP2), SU6656, UCS15A, and combinations thereof.

10. The method of claim 8 or claim 9, wherein at least one of the one or more modulators of SRC pathway is HGF.

11. The method of claim 8 or claim 9, wherein at least one of the one or more modulators of SRC pathway is IL-1β.

12. The method of claim 8 or claim 9, wherein at least one of the one or more modulators of SRC pathway is FGF-1.

13. The method of any one of claims 8 to 12, wherein the method comprises incubating differentiated cells in a medium comprising a plurality of modulators of SRC pathway.

14. The method of claim 13, wherein the plurality of modulators comprise a combination of two or more of HGF, IL-1β and FGF-1.

15. The method according to any one of claims 8 to 14, wherein the method comprises incubating the differentiated cells in a medium comprising one or more modulators of RhoA and/or ROCK pathway and one or more modulators of SRC pathway.

16. The method according to any one of claims 8 to 14, wherein the method comprises incubating the differentiated cells separately or sequentially in a medium comprising one or more modulators of RhoA and/or ROCK pathway and in a medium comprising one or more modulators of SRC pathway.

17. The method according to any one of claims 8 to 14, wherein the method comprises incubating the differentiated cells in a medium comprising one or more modulators of RhoA and/or ROCK pathway before incubating the cells in a medium comprising one or more modulators of SRC pathway.

18. The method according to any one of claims 8 to 14, wherein the method comprises incubating the differentiated cells in a medium comprising one or more modulators of SRC before incubating the cells in a medium comprising one or more modulators of RhoA and/or ROCK pathway.

19. The method according to any one of claims 9 to 14, wherein the one or more modulators of SRC pathway include one or more agonists or partial agonists or reverse antagonists of SRC pathway.

20. The method according to any one of claims 9 to 14, wherein the one or more modulators of SRC pathway are capable of inducing de-differentiation of the differentiated cells into the progenitor cells.

21. A method for producing a progenitor cell capable of being differentiated into a plurality of different cell types, said method comprising incubating differentiated cells in a medium comprising an amount of one or more modulators of SRC pathway for a time and under conditions sufficient to produce a progenitor cell that is capable of being differentiated into a plurality of different cell types.

22. The method of claim 21, wherein the one or more modulators of SRC pathway are selected form the group consisting of transforming growth factor (TGF), transforming growth factor beta-1 (TGF-β1), nerve growth factor-βa (NGFβ), interleukin 1-β (IL-1β), Fibroblast growth factor-1 (FGF-1), fibroblast growth factor-2 (FGF-2), hepatocyte growth factor (HGF), neurotrophin 3 (NT3), semaphorin (SEMA), semaphorin-3A (SEMA-3A), platelet-derived growth factor (PDGF), platelet-derived growth factor B-chain (PDGF-B), 4-amino-5-(4-methylphenyl)-7-(t-butyl)pyrazolo (3,4-d) pyrimidine, (PP1), 4-Amino-5-(4-chlorophenyl)-7-(t-butyl)pyrazolo[3,4-d]pyrimidine (PP2), SU6656, UCS15A, and combinations thereof.

23. The method of claim 21 or claim 22, wherein at least one of the one or more modulators of SRC pathway is selected form the group consisting of: HGF, IL-1β and FGF-1.

24. The method of any one of claims 21 to 23, wherein the method comprises incubating differentiated cells in a medium comprising a plurality of modulators of SRC pathway.

25. The method of claim 24, wherein the plurality of modulators comprise a combination of two or more of HGF, IL-1β and FGF-1.

26. The method according to any one of claims 1 to 25, wherein said method further comprises detaching the cells.

27. The method of claim 26, comprising detaching the cells following incubation of the differentiated cells with one or more modulators of RhoA and/or ROCK pathway and/or one or more modulators of SRC pathway.

28. The method of claim 26 or claim 27, comprising detaching the cells by incubating the cells in a medium comprising a protease and/or incubating cells expressing one or more protease activated receptors (PARs) with one or more PAR ligands.

29. The method of claim 28, wherein a PAR is selected from the group consisting of PAR-1, PAR-2, PAR-3 and PAR4.

30. The method according to any one of claims 26 to 29, comprising producing progenitor cells capable of being differentiated into a plurality of different cell types until re-attachment or adherence or contact of the cells to the culture vessel and/or to each other.

31. The method according to any one of claims 1 to 30, further comprising incubating differentiated cells in a low-serum medium comprising a low serum concentration and without supplementation of factors normally present in serum, for a time and under conditions sufficient to produce a progenitor cell that is capable of being differentiated into a plurality of different cell types.

32. The method of claim 31, wherein the low-serum medium does not exceed about 3% (v/v) total serum concentration.

33. The method of claim 31 or 32, comprising incubating the differentiated cells in low-serum medium for at least about 2 days and not exceeding about 10 days.

34. The method according to any one of claims 1 to 33, wherein the method further comprises incubating or maintaining or culturing the cells in high cell-density conditions.

35. The method of claim 34, wherein incubating or maintaining or culturing the cells in high cell-density conditions comprising incubating or maintaining or culturing the cells until confluence or cell-to-cell contact is achieved.

36. The method of claim 34 or claim 35, wherein the high cell-density conditions comprise a minimum density between about 1500 cells/mm² plating surface area to about 10,000 cells/mm² plating surface area.

37. The method according to any one of claims 34 to 36, comprising incubating or maintaining or culturing the cells in high cell-density conditions after incubating the differentiated cells in a medium comprising an amount of one or more modulators of RhoA and/or ROCK pathway and/or an amount of one or more modulators of SRC pathway.

38. The method according to any one of claims 34 to 36, comprising incubating or maintaining or culturing the cells in high cell-density conditions at the same time as incubating the differentiated cells in a medium comprising an amount of one or more modulators of RhoA and/or ROCK pathway the cells and/or an amount of one or more modulators of SRC pathway.

39. The method according to any one of claims 34 to 36, comprising incubating or maintaining or culturing the cells in high cell-density conditions before incubating the differentiated cells in a medium comprising an amount of one or more modulators of RhoA and/or ROCK pathway the cells and/or an amount of one or more modulators of SRC pathway.

40. The method according to any one of claim 1 or 39, wherein the differentiated cells are mammalian fibroblasts or chondrocytes.

41. The method according to any one of claims 1 to 40, wherein the progenitor cells are capable of being differentiated into a cell type selected from the group consisting of a cardiac cell, a cardiomyocyte, a cardiac muscle cell, a cardiac fibroblast, a skin cell, an epidermal cell, a keratinocyte, a melanocyte, an epithelial cell, a neural cell, a dopaminogenic cell, a glial cell, a Schwann cell, an astrocyte, an oligodendrocyte, a microglial cell, a blood cell, a lymphocyte, a T cell, a B cell, a macrophage, a monocyte, a dendritic cell, a Lagerhans cell, an eosinophil, an adipocyte, an osteoclast, an osteoblast, an endocrine cell, a β-islet cell, an insulin secreting cell, an endothelial cell, an epithelial cell, a granulocyte, a hair cell, a mast cell, a myoblast, a Sertoli cell, a striated muscle cell, a zymogenic cell, an oxynitic cell, a brush-border cell, a goblet cell, a hepatocyte, a Kupffer cell, a stratified squamous cell, a pneumocyte, a parietal cell, a podocyte, a synovial cell, a serosal cell, a pericyte, a chondrocyte, an osteocyte, a Purkinje fiber cell, a myoepithelial cell, a megakaryocyte, and combinations thereof.

42. The method according to any one of claims 1 to 41, further comprising isolating progenitor cells capable of being differentiated into a plurality of different cell types.

43. A progenitor cell capable of being differentiated into a plurality of different cell types, wherein said cell is a product of the method according to any one of claims 1 to 42.

44. A progenitor cell isolated by the method of claim 42, wherein the progenitor cell is capable of being differentiated into a plurality of different cell types.

45. A method for producing a differentiated cell comprising incubating a progenitor cell according to claim 43 or 44 for a time and under conditions sufficient to produce a differentiated cell.

46. The method according to claim 45, comprising incubating the progenitor cell in vitro.

47. The method according to claim 45, comprising incubating the progenitor cell in vivo.

48. The method according to any one of claims 45 to 47, further comprising isolating the differentiated cell.

49. A differentiated cell, wherein said cell is a product of the method according to any one of claims 45 to 48.

50. A cell culture comprising a plurality of cells according to any one of claim 43, 44 or 49.

51. A method for producing and/or repairing and/or regenerating a tissue or an organ comprising incubating a progenitor cell, differentiated cell or cell culture according to any one of claim 43, 44, 49 or 50 for a time and under conditions sufficient to produce and/or repair and/or regenerate one or more tissues or organs from the cell or cell culture.

52. The method according to claim 51 comprising culturing or perfusing the cells or cell culture onto or into a biocompatible scaffold or matrix for a time and under conditions sufficient for the cell or cell culture to produce and/or repair and/or regenerate one or more tissues or organs.

53. The method according to claim 52, wherein the scaffold or matrix comprises a decellularized tissue or organ or a derivative thereof.

54. The method according to claim 52, wherein the scaffold or matrix comprises collagen and/or proteoglycan.

55. The method according to any one of claims 51 to 54, further comprising incubating the progenitor cell, differentiated cell or cell culture in the presence of at least one growth factor or mitogen or a morphogen or a functional fragment thereof or nucleic acid encoding said growth factor, mitogen, morphogen or functional fragment thereof.

56. The method according to any one of claims 51 to 55, further comprising providing the progenitor cells an agent selected from the group consisting of a neuropeptide Y (NPY), a fragment of neuropeptide Y, a variant of neuropeptide Y, a compound capable of inducing expression of a gene encoding a neuropeptide Y protein or fragment or variant thereof, a cell that produces a neuropeptide Y and an agonist or antagonist of a neuropeptide Y receptor, a neurotrophin, a fragment of a neurotrophin, a compound capable of inducing expression of a neurotrophin gene, and/or an agonist or antagonist of a receptor for a neurotrophin, a neuregulin, a fragment of a neuregulin, a compound capable of inducing expression of a neuregulin gene, and an agonist or antagonist of a receptor for neuregulin, and combinations thereof, wherein said agent induces regeneration, repair or building of a tissue or organ.

57. The method of claim 56, wherein the agent in provided to the progenitor cells in situ at the site of tissue and/or organ injury.

58. The method according to any one of claims 51 to 57, further comprising isolating the tissue(s) or organ(s) and optionally, providing the tissue(s) or organ(s) to a subject in need thereof.

59. An isolated tissue or organ produced, repaired or regenerated by the method according to any one of claims 51 to 58.

60. A pharmaceutical composition comprising a progenitor cell, differentiated cell or cell culture according to any one of claim 43, 44, 49 or 50 and a pharmaceutically acceptable carrier.

61. A method of prophylaxis or treatment of a condition requiring organ or tissue formation and/or regeneration and/or repair in a subject, said method comprising administering or transplanting or grafting to said subject an effective amount of the progenitor cell, differentiated cell, cell culture, tissue or organ according to any one of claim 43, 44, 49, 50 or 59 thereby preventing or treating the condition in the subject.

62. A method of prophylaxis or treatment of a condition in a subject that is normally alleviated by administering, grafting or transplanting stem cells or a tissue or organ to a subject, said method comprising administering or transplanting or grafting to said subject an effective amount of the progenitor cell, differentiated cell, cell culture, tissue or organ according to any one of claim 43, 44, 49, 50 or 59 thereby preventing or treating the condition in the subject.

63. The progenitor cell, differentiated cell, cell culture, tissue or organ according to any one of claim 43, 44, 49, 50 or 59 for use as a medicament.

64. The progenitor cell, differentiated cell, cell culture, tissue or organ according to any one of claim 43, 44, 49, 50 or 59 for use as a medicament to stimulate or enhance tissue or organ formation and/or regeneration and/or repair.

65. The progenitor cell, differentiated cell, cell culture, tissue or organ according to any one of claim 43, 44, 49, 50 or 59 for use as a medicament in the treatment or prophylaxis of one or more conditions normally alleviated by administering stem cells or tissue or organ derived from stem cells to a subject or by grafting stem cells or tissue or organ derived from stem cells into a subject or by transplanting stem cells or tissue or organ derived from stem cells into a subject.

66. Use of a progenitor cell, differentiated cell, cell culture, tissue or organ according to any one of claim 43, 44, 49, 50 or 59 in the preparation of a medicament for stimulating or enhancing tissue or organ formation and/or regeneration and/or repair in a subject.

67. A composition comprising a progenitor cell, differentiated cell, cell culture, tissue or organ according to any one of claim 43, 44, 49, 50 or 59 and a biocompatible scaffold or matrix.

68. A kit for regenerating and/or repairing and/or building a tissue or an organ, wherein said kit comprises:
(i) a progenitor cell, differentiated cell or cell culture according to any one of claim 43, 44, 49 or 50;
(ii) a biocompatible scaffold or matrix;
(iii) optionally, at least one growth factor or mitogen or functional fragment thereof or nucleic acid encoding said growth factor, mitogen, morphogen or functional fragment thereof;
(iv) optionally, an agent selected from the group consisting of a neuropeptide Y (NPY), a fragment of neuropeptide Y, a variant of neuropeptide Y, a compound capable of inducing expression of a gene encoding a neuropeptide Y protein or fragment or variant thereof, a cell that produces a neuropeptide Y and an agonist or antagonist of a neuropeptide Y receptor, a neurotrophin, a fragment of a neurotrophin, a compound capable of inducing expression of a neurotrophin gene, and/or an agonist or antagonist of a receptor for a neurotrophin, a neuregulin, a fragment of a neuregulin, a compound capable of inducing expression of a neuregulin gene, and an agonist or antagonist of a receptor for neuregulin, and combinations thereof; and
(iv) directions for preparing, maintaining and/or using the cells or the scaffold material or matrix including any cell culture or tissue or organ derived therefrom.
